(19) United States
(12) Patent Application Publication
(10) Pub. No.: US 2004/0106539 A1
(43) Pub. Date: Jun. 3, 2004

(54) Agents for the treatment of viral infections

(76) Inventors: Ulrich Schubert, Ulhlstadt (DE); Hans Will, Hamburg (DE); Uwe Tessmer, Hamburg (DE); Husseyin Strma, Wiesloch (DE); Alexij Prassolow, Hamburg (DE); Eveleyn Schubert, Uhlstadt (DE); Heinz Hohenberg, Hamburg (DE); Reinhardt Welker, Reutlingen (DE)

Correspondence Address: FULBRIGHT & JAWORSKI, LLP, 666 FIFTH AVE, NEW YORK, NY, 10103-3198, US

(21) Appl. No.: 10/398,993

(22) PCT Filed: Oct. 11, 2001
(86) PCT No.: PCT/DE01/03908

(30) Foreign Application Priority Data
Oct. 12, 2000 (DE) 100 51 716.1
Oct. 3, 2001 (DE) 101 49 398.3

(51) Int. Cl. A61K 38/00

(52) U.S. Cl. 514/2

(57) ABSTRACT

The invention relates to agents for the treatment of viral infections, in particular, infections with hepatitis and retro-viruses. Said agents inhibit the release, maturation and replication of both retro-viruses and also hepatitis viruses. In the example of human immune deficiency virus (HIV) and hepatitis-B viruses it has been shown that proteasome inhibitors block the release of virus particles and the infectiousness of the released viral particles and thus the reproduction of the viruses. The proteasome inhibitors affect the activities in the ubiquitin/proteasome pathway, in particular the enzymatic activities of the 26S and the 20S proteasome complexes. The application for the above invention lies in anti-retroviral therapy, particularly the treatment of HIV infections and AIDS and in the anti-viral therapy of hepatitis infections, in particular the treatment of acute and chronic HBV and HCV infections and the associated liver carcinomas.

The invention relates to compositions for treating viral infections, in particular infections with hepatitis viruses and retroviruses. The invention relates to compositions, which contain proteasome inhibitors as active compound, for inhibiting the release, the maturation, the infectivity and thus the replication of both retroviruses and hepatitis viruses. On the basis of the example of type 1 and 2 human immunodeficiency viruses (HIV-1/HIV-2) it is demonstrated that said compositions block both processing of HIV-1 and HIV-2 Gag proteins and release of HIV-1 and HIV-2 virus particles and also the infectivity of the released virus particles and thereby HIV virus replication.

Furthermore, said compositions may be used for the treatment, therapy and inhibition of a viral hepatitis. It is demonstrated that the applications of said compositions result in the release of noninfectious hepatitis viruses from infected cells. Said compositions may therefore limit the spread of an acute infection with hepatitis viruses. Furthermore, the compositions are less toxic for nonproliferating hepatocytes than for nonparenchymal liver cells and liver carcinoma cells. Thus, said compositions are suitable for a preferred destruction of liver carcinoma cells in patients and animals infected with HBV (hepatitis B viruses, list of abbreviations after the examples) and HCV (hepatitis C viruses). The compositions are various substance classes which share the ability to inhibit the ubiquitin-proteasome system. Specifically, said compositions are characterized in that said substances, the proteasome inhibitors, inhibit the activity of the major cellular protease, i.e. the proteasome, in the treated cells. On the basis of the example of the duck hepatitis virus as well as the human hepatitis B virus, it is demonstrated that the application of proteasome inhibitors drastically reduces the release of infectious duck hepatitis viruses and human hepatitis B viruses from already infected hepatocytes (demonstrated, by way of example, for the duck hepatitits virus). Said proteasome inhibitors can therefore suppress viremia in the case of both a new infection and chronic infections with hepatitis viruses and successful virus elimination by the endogenous immune system and/or by known compositions having a similar or different action can be enhanced. Using said compositions, namely the proteasome inhibitors, can prevent, reduce or reverse the consequences of a HBV and HCV infection, such as, for example, liver damage of differing degrees of severity up to the frequently fatal fulminant hepatitis, development of liver cirrhosis/fibrosis and of liver carcinoma. Fields of application for these inventions are therefore antiretroviral therapy and the prevention of infections with immunodeficiency-causing antiviruses, especially the acquired immunodeficiency in animals and humans, in particular of HIV-1/HIV-2 infections and AIDS, as well as antiviral therapy of hepatitis virus infections, in particular for preventing acute and chronic HBV and HCV infections from being established and maintained.

### CHARACTERISTICS OF THE KNOWN PRIOR ART

0. Introduction

Since the beginning of the 80s, the acquired immunodeficiency syndrome (AIDS) pandemic has confronted millions of HIV-infected humans with an insidious, multisystemic and ultimately, up until now, incurable disease. Currently, there is neither a known effective strategy for vaccination against HIV nor are any other mechanisms for stimulating a still poorly understood natural immunity to an HIV infection applicable for broad application. Various antiretroviral medicaments are used for therapy of an already established HIV infection or for protecting against systemic manifestation of an HIV infection immediately after virus uptake. Said medicaments are essentially substances which inhibit the viral enzymes reverse transcriptase (RT) and protease (PR). Apart from'the problem of intolerance, the main limitation of said medicaments is the enormous, up to 10⁶ times higher rate of mutation of HIV (compared to replication of human DNA). The polymorphism resulting therefrom leads inevitably and after a relatively short time to the appearance of HIV mutants which are resistant to individual or even combined anti-HIV therapeutics, in particular in HAART therapy (highly active antiretroviral therapy—patent publication WO 00/33654). The aim of future HIV research is therefore the identification of cellular targets for antiretroviral therapy. This relates to cellular factors, enzymes or complex mechanisms which are essential for HIV replication in the host cell and can be manipulated selectively without substantially impairing the overall vitality of the organism. This demand is fulfilled by the surprise finding described in the present invention, namely that said compositions, the proteasome inhibitors, inhibit late processes in HIV-1 and HIV-2 replication and thereby prevent the release and formation of infectious viral progeny.

The infection with hepatitis B viruses (affecting approx. 5% of the world's population) and hepatitis C viruses (affecting approx. 3% of the world's population) is, together with the HIV/AIDS problem, one of the big problems of world health. Infections with HBV or HCV frequently result in a chronic virus carrier state. The symptoms of the infections include inflammations of the liver of varying degrees of severity up to liver failure (fulminant hepatitis), an increased risk of developing a liver cirrhosis and fibrosis and the development of liver carcinomas. New infections with HBV can be prevented relatively efficiently, but not completely due to the existence of vaccination failures and immune escape variants, by prophylactic immunization. There is as yet no protection provided by vaccination against a new infection with HCV. Despite the multiplicity of medicaments for the therapy of a chronic HBV and HCV infection, all of which are encumbered with side effects and essentially comprise cytokines (interferon alpha and variants thereof) and nucleoside analogs, it is not possible as yet to carry out a satisfactory therapy of the majority of chronic HBV and HCV carriers, since either the patients do not respond to the medicaments or there is only a short-term improvement and the virus usually cannot be eliminated completely by the treatment. Likewise, passive administration of HBV-specific neutralizing antibodies and/or nucleoside analogs or of other medicaments in liver transplant patients usually does not prevent de novo infection of the transplanted liver. Immune suppression in patients with hepatitides in remission can reactivate latent viruses. The main problem in the case of nucleotide analogs is the high rate of mutation both of HBV and HCV, resulting in the development of medicament-resistant viral strains during treatment. In order to avoid the problems of the previously available antiviral and therapeutic compositions for hepatitis B and C, novel therapeutic approaches are required which, similar to antiretroviral therapy, influence conserved cellular factors which are essential for the propagation of said viruses in the host cell. The present invention describes such compositions. In fact, this relates to the surprise finding that proteasome inhibitors, similar to the novel effects on retroviruses, likewise prevent the production of infectious hepatitis viruses and, at the same time, induce the death of liver tumor cells. Said compositions, the proteasome inhibitors, are, due to the possibility of preferably transporting said compositions into the liver, particularly suitable for selective therapy of viral liver disorders and liver carcinomas (list of references after the exemplary embodiments).

1. Function of the Ubiquitin/Proteasome Pathway

Proteasomes are multicatalytic and multi-subunit enzyme complexes which represent approx. 1% of the total cell protein and occur as the major proteolytic component in the nucleus and cytosol of all eukaryotic cells. The essential function of proteasomes is the proteolysis of misfolded or nonfunctional proteins or of usually regulatory proteins designed for rapid degradation. Another function of proteasomal degradation of a multiplicity of cellular or viral proteins is the generation of peptide ligands for major histocompatibility (MHC) class I molecules which are required for T-cell-mediated immune response (for a review, see Rock and Goldberg, 1999).

Proteasome targets are usually marked for proteasomal degradation by attachment of oligomeric forms of ubiquitin (Ub). Ub is a highly conserved protein of 76 amino acids in length, which is covalently coupled to target proteins via isopeptide binding between the COOH terminus and the ε-NH₂ group of lysine side chains, either to the target protein or to Ub molecules already attached to said target protein. The conjugation of Ub molecules results in the formation of “poly-Ub chains”. In general, multimers of four Ub molecules are required in order to function as a signal for degradation by the proteasome. Ubiquitination itself is reversible, and Ub molecules can be removed again from the target molecule by a multiplicity of Ub hydrolases. The connection between ubiquitination of target proteins and proteasomal proteolysis is generally referred to as ubiquitin-proteasome system (UPS) (for a review, see Hershko and Chiechanover, 1998; Baumeister et al., 1998).

The 26S proteasome is a 2.5 megadalton (MDa) multienzyme complex which consists of approx. 31 subunits, (for a review, see Voges et al., 1999). The proteolytic activity of the proteasome complex is provided by a core structure, the 20S proteasome. The 20S proteasome forms a complicated multienzyme complex consisting of 14 nonidentical proteins (with molecular weights ranging from 21 to 31 kDa), which is arranged in two α and two β rings in an αββα order (for a review, see Voges et al., 1999). The substrate specificity of the 20S proteasome comprises three essential proteolytic activities: trypsin-, chymotrypsin- and postglutamyl peptide-hydrolyzing (PGPH), or else caspase-like, activities which are located in the β subunits X, Y and Z. The enzymic activities of the 20S proteasome are controlled by attachment of the 19S regulatory subunits which together form the active 26S proteasome particle. The 19S regulatory subunits are involved in the recognition of polyubiquitinated proteins and in the unfolding of target proteins. The 26S proteasome activity is ATP-dependent and degrades almost exclusively only polyubiquitinated proteins. The catalytically active β subunits of the 20S proteasome (X, Y and Z) may be replaced by γ-interferon-inducible MHC-encoded subunits which then form the “immunoproteasome” (Gaczynska et al., 1993).

1.1 Importance of the Ubiquitin-Proteasome System in the Pathogenesis of Clinically Relevant Disorders

The close linkage of the UPS (ubiquitin-proteasome system) to cellular mechanisms explains the importance of said system for numerous pathological mechanisms of which only some are known to date (for a review, see Schwartz and Ciechanover, 1999). The UPS performs a central function in disorders of the immune system. Firstly, the 26S proteasome complex is the major protease in MHC-I antigen processing and, secondly, it is possible for γ-interferon-inducible catalytic β subunits as well to manipulate the activity of the proteasome itself. Many inflammatory and immunological disorders are associated with the transcription factor NF-κB which regulates various gene functions during the immune response. The activation of NF-κB, which is controlled by ubiquitination and specific cleavage of a precursor protein by the proteasome, results in increased expression of various cytokines, adhesion molecules, inflammatory and stress response proteins and immunoreceptors (for a review, see Chiechanover et al., 2000; Schwartz and Ciechanover, 1999).

1.2. Proteasome Inhibitors

Various substance classes are known as proteasome inhibitors. They are, on the one hand, chemically modified peptide aldehydes such as the tripeptide aldehyde N-carbobenzoxyl-L-leucinyl-L-leucinyl-L-leucinal (zLLL) which is also referred to as MG132 and the boric acid derivative MG232 which is about 10 times more effective. zLLL and derivatives derived therefrom block the proteasome reversibly by forming a transient hemiacetal structure with the catalytically active threonine-hydroxyl side chain in position 1 of the β subunit of the 26S proteasome (for a review, see Coux et al., 1996). Similarly to zLLL, another class of modified peptides has been described as proteasome inhibitors, peptide vinyl sulfones (Bogyo et al., 1997).

Naturally occurring substances isolated from microorganisms are lactacystin (LC) (Fenteany et al., 1995), from streptomycetes, and epoxomicin, from actinomycetes (Meng et al., 1999a,b). LC is a highly specific and effective proteasome inhibitor which irreversibly inactivates the proteasome by transesterification and alkylation of the threonine side chain in the β subunit (Fenteany et al., 1995). LC is therefore an irreversible, covalently acting proteasome inhibitor which primarily blocks the chymotrypsin- and trypsin-like activities of the 26S proteasome particle (Fenteany et al., 1995). LC has no peptide base structure but consists of a γ-lactam ring, a cysteine and a hydroxybutyl group. LC itself does not inhibit the proteasome. Rather, the N-acetyl-cysteine residue is hydrolyzed in aqueous solution. This results in the formation of a Clastolactacystin β-lactone. This lactone structure is capable of penetrating cell membranes. Absorption into the cell is followed by a nucleophilic attack of the β-lactone ring and subsequent transesterification of the threonine¹ hydroxyl group of the β subunit.

Another proteasome inhibitor is the naturally occurring epoxyketone epoxomicin. With respect to specificity for the 26S proteasome and efficacy, epoxomicin is the as yet most effective of all known naturally occurring proteasome inhibitors (Meng et al., 1999a,b). Furthermore, epoxomicin is distinguished by a comparatively low toxicity in cell cultures (Hanada et al., 1992).

Another very potent class of synthetic proteasome inhibitors are boric acid peptide derivatives, in particular the compound pyranozyl-phenyl-leuzinyl-boric acid, referred to as “PS-341”. PS-341 is very stable under physiological conditions and is bioavailable after intravenous administration (Adams and Stein, 1996; Adams et al., 1998). Boric acid peptide derivatives are generally known as inhibitors of a large variety of eukaryotic proteases such as, for example, thrombin, elastase, dipeptidyl protease IV (for a review, see Adams and Stein, 1996). The particular effectiveness of PS-341 as proteasome inhibitor is provided by the very stable bond between the boric acid group and the hydroxyl group of the catalytically active side chain of Thr¹ in the active β subunit of the 20S proteasome, with an inhibition constant (Ki) of 0.6 nM (Adams and Stein, 1996). Up until now, the proteasome is the only known cellular protease influenced by PS-341.

Other boric acid-peptide derivatives similar to PS-341 have been described as proteasome inhibitors, such as, for example, benzoyl-phenylalanine-boric acid-leucine (Gardner et al., 2000). Likewise, a highly potent proteasome inhibitor, referred to as “PS-273” (morpholino-CONH—(CH-naphthyl)-CONH—(CH-isobutyl)-B(OH)₂) has been described which has a base structure similar to that of PS-341 but is, due to a morpholino structure at the N-terminus, more hydrophobic and thus can presumably penetrate membranes more readily than PS-341 (Adams et al., 1999).

Clinical Administration of Proteasome Inhibitors

The proteasome complex carries out essential cellular functions and is indispensable for cell vitality. Permanent inhibition of proteasome activity can therefore result in changes in cell cycle regulation, transcription, the entire cellular proteolysis and in MHC-I antigen processing (for a review, see Hershko and Ciechanover, 1998). A complete inhibition of proteasome activity generally leads to cell cycle arrest and cell death. A permanent inhibition of all enzymic activities of the proteasome is incompatible with the viability of a cell and thus of the entire organism.

However, novel proteasome inhibitors acting in a reversible manner have been shown to inhibit selectively individual proteolytic activities of the 26S proteasome, without influencing other cellular proteases in the process. The cytotoxicity of such inhibitors is therefore substantially lower in comparison with proteasome inhibitors acting in a relatively unspecific manner, such as, for example, the peptide aldehyde zLLL. This fact allows both the in vivo administration of such novel proteasome inhibitors (Meng et al., 1999a) and the establishment of permanent cell lines which tolerate relatively high concentrations of proteasome inhibitors (Glas et al., 1998; Geier et al., 1999).

The claim that particular proteasome inhibitors in a particular dose regime can be tolerated in vivo has been demonstrated several times. Thus, for example, the selection of mouse thymus cell lines which tolerate the continuous presence of 10 μM of the proteasome inhibitor z-leucinyl-leucinyl-leucinyl-vinyl sulfone (NLVS) and have normal cell growth and cell metabolism with simultaneously limited MHC-I antigen presentation has been described (Glas et al., 1998). Similar results were obtained using the very potent proteasome inhibitor LC which was tolerated at up to 6 μM in cell culture (Geier et al., 1999).

Epoxomicin, an epoxy β-aminoketone-modified peptide, was isolated from actinomycetes as a completely novel class of proteasome inhibitors (Hanada et al., 1992). Epoxomicin is highly cytotoxic for various in vitro-cultured tumor cell lines and exhibited in vivo inhibitory activity against melanoma and leukemia model tumors in the mouse model (Hanada et al., 1992).

The importance of proteasome inhibitors as a novel therapeutic principle has received increasing attention in recent years, in particular in the treatment of cancer and inflammatory disorders (for a review, see Murray and Norbury, 2000; Rivett and Gardner, 2000—Category XXX=references after priority date). The use of proteasome inhibitors for broad clinical administration in humans has not been permitted as yet. However, an increasing number of reports in the relevant literature state that, recently, the pharmaceutical industry has been working intensively on the development of new medicaments based on proteasome inhibitors tolerated in vivo. A few examples should be mentioned in this connection: after the takeover of ProScript, Inc., Millennium Pharmaceuticals, Inc. (Cambridge, Mass. 02139, USA) are working on the development of proteasome inhibitors for anti-inflammatory, immunomodulatory and antineoplastic therapies, in particular on boric acid derivatives of dipeptides. The compounds PS-341 (Gardner et al., 2000 Category XXX), PS-519 and PS-273 (Adams et al., 1998, 1999) play a particular part here.

The oral administration of PS-341 has an anti-inflammatory effect on streptococci-induced polyarthritis and inflammation of the liver in the rat model (Palombella et al., 1998). In the mouse model, PS-341 exhibits antineoplastic action on lung carcinoma and has, in addition, an additive effect in connection with cytostatics (Teicher et al., 1999). In vitro experiments demonstrate very good effectiveness against solid human ovarian and prostate tumor cells (Frankel et al., 2000). Phase I clinical studies on PS-341 demonstrate good bioavailability and pharmacokinetic behavior (Lightcap et al., 2000 Category XXX).

Up until now, PS-341 is the only clinically tested proteasome inhibitor. For this purpose, phase I and phase II clinical studies in patients with different cancers such as, for example, hematological malignancies as solid tumors have already been completed. Millennium Pharmaceuticals Inc. have presented the relevant information in various press announcements:

For example, results of phase II clinical studies in multiple myeloma patients were reported (press announcement by Millennium Pharmaceuticals, Inc. of 03.01.01: “Millennium Initiates Phase II Clinical Trials of LDP-341 in Multiple Myeloma.” Published on the web page http:biz.yahoo.com/prnews/010301/neth003.html, Category XXX).

First preclinical studies on the action of proteasome inhibitors PS-341 as a novel anti-cancer therapy in myeloma patients was presented at the 42nd Meeting of the American Hematological Society, December 2000, San Francisco, Calif., USA (press announcement by Millennium Pharmaceuticals, Inc. of Dec. 4, 2000: Millennium's LDP(PS)-341 inhibits growth and induces death of cancer cells, appears to overcome chemotherapy resistance. Published on the web page http:biz.yahoo.com/prnews/010301/neth003.html, Category XXX).

At the meeting of the American Society for Clinical Oncology, May 2000, data from phase I clinical studies with PS-341 in patients with advanced malignant tumors (including melanoma, kidney carcinoma, lung carcinoma, prostate cancer, cancer of the ovaries, cancer of the bladder, cervical cancer, endometrials and tumors of the gall bladder) were presented. No dose-limiting toxicity due to the treatment with PS-341 was observed. Owing to the astonishing action of PS-341 with respect to antineoplastic action and induction of apoptosis, therapeutic effects in various tumor patients were observed (press announcement by Millennium Pharmaceuticals, Inc. of May 23, 2000: “Millennium presents clinical trial data on LDP-341 for advanced malignancies.” Published on the web page http://www.mlnm.com.releases.pr052300_{—}1.shtml).

Further information on the clinical protocol of phase I studies in patients with advanced tumors (solid tumors as well as lymphomas) who had no longer responded to standard chemotheraphy were published on line on “CancerNet” (“Phase I study of PS-341 in patients with advanced solid tumors or lymphomas”. Published on Sep. 11, 2000 on the web page http://cancernet.nci.nih.gov/, Category XXX).

Results of phase I clinical studies on PS-341 in patients with acute leukemia, myelodysplastic syndrome and chronic myelidic leukemia were reported, in particular the synergistic action of PS-341 with standard chemotherapeutics (press announcement by Millennium Pharmaceuticals, Inc. of Nov. 09, 2000: “Phase I study of PS-341 in acute leukemias, myelodysplastic syndromes and chronic myeloid leukemia in blast phase.” Published online in Leukemia Insights Newsletter on the web page http://www3.manderson.org/leukemia/insight/letter52.htm 1).

It was possible to show in a mouse model that the proteasome inhibitor PS-519 (a β-lactone derivative), developed by Millennium Pharmaceuticals, Inc., exerts a strong anti-inflammatory action, namely on both the delayed and the oversensitive inflammatory response. In lower doses, PS-519 is also effective in combination with steroids. PS-519 was therefore proposed as a new medicament for the treatment of asthma (Elliott et al., 1999). Another application for PS-519 arises in the infarct model: PS-519 dramatically reduces the inflammatory response after cerebral injuries. According to this, PS-519 likewise appears to be an interesting pharmaceutical for the treatment of stroke (Phillips et al., 2000, Category XXX).

Since proteasome inhibitors affect an essential pathway in cell metabolism, a strict dose regime is necessary in order to suppress toxic side effects. As part of the development of in vivo-tolerated proteasome inhibitors, various peptide-boric acid derivatives have been tested which exhibited anti-tumor action both in cell culture and in the animal model (Adams and Stein, 1996: Adams et al., 1998, 1999, 2000). PS-341 has a selective cytotoxic activity in vitro against a broad spectrum of human tumor cell lines (Adams et al., 1999). This activity is linked to the accumulation of p21 and cell cycle arrest in the G2-M phase with subsequent apoptosis (Adams et al., 1999). Direct injection of PS-341 destroyed 70% of the tumors studied in the mouse model. After intravenous administration of PS-341, the substance spread to all organs and tissues and had antineoplastic activity in human xenograft models (Adams et al., 1999).

The use of proteasome inhibitors of any substance class with the aim of blocking viral infections has not been reported previously. A claim of blocking/inhibiting the replication cycle of HIV-1, HIV-2 or other lentiviruses or even of treating AIDS patients by means of reagents influencing, regulating or blocking the UPS has not been made in the literature as yet.

1.3. Connection Between the Ubiquitin/Proteasome Pathway and the Replication Cycle of Retroviruses

1.3.1. Biology of Retroviruses

The family of retroviruses which also includes the human immune deficiency viruses (HIV) belongs to the large group of eukaryotic retrotransposable elements (for a review, see Doolittle et al., 1990). Said elements are distinguished by the ability to transcribe RNA genomes into DNA intermediates by using the enzyme reverse transcriptase. Retroviruses are divided into five subfamilies: (i) spumaviruses; (ii) mammalian type C oncoviruses; (iii) BLV (bovine leukemia virus)/HTLV (human T-cell leukeumia virus) leukemia viruses; (iv) a heterogeneous group of RSV (Rous sarcoma virus), type A, B and D viruses; and (v) lentiviruses (for a review, see Doolittle et al., 1990).

Lentiviruses replicate predominantly in lymphocytes and fully differentiated macrophages and usually cause long-lasting and usually incurable diseases. Retroviruses contain at least three characteristic genes: gag (group-specific antigen), pol (polymerase) and env (envelope proteins). Apart from structural and enzymatically active viral proteins, various retroviruses encode additional, usually small proteins with regulatory functions. The lentivirus subfamily includes, in addition to HIV, SIV (simian immunodeficiency virus), EIAV (equine infectious anemia virus), BIV (bovine immunodeficiency virus), FIV (feline immunodeficiency virus) and Visna virus. HIV in turn is divided into the two subtypes HIV-1 and HIV-2 (for a review, see Doolittle et al., 1990).

1.3.2. HIV Replication Cycle

The HIV replication cycle starts with the virus binding to various cell receptors among which the glycoprotein CD4 acts as the primary receptor and various cell-specific chemokine receptors act as co-receptors, after binding to CD4. After the virus has entered, the viral RNA genome is transcribed by means of reverse transcriptase (RT), RNase H and polymerase into double-stranded DNA which, in association with the preintegration complex, is then transported into the nucleus and incorporated as provirus genome into chromosomal DNA by means of viral integrase. After transcription and translation, Gag/Gag-Pol polyproteins and envelope proteins are transported to the cell membrane where virions are being assembled. After budding and detachment, virus particles mature due to proteolytic processing of said Gag/Gag-Pol polyproteins (for a review, see Swanstrom and Wills, 1997).

1.3.3. Assembly, Release and Maturation of HIV Particles

The main components of HIV structural proteins are translated in the form-of three polyproteins: Gag and Gag-Pol for the inner core proteins and viral enzymes and Env for proteins of the viral envelope proteins. In the case of HIV-1, complete proteolytic processing of the Gag polyprotein Pr55 results in the formation of the matrix (MA), capsid (CA) and nucleocapsid (NC) and of the C-terminal p6^{gag} protein. In general, HIV-1 virions are detached from the plasma membrane as mature noninfectious virus particles, this process being referred to as virus budding. Immediately after or else during budding, proteolytic processing of Gag and Gag-Pol polyproteins commences with the activation of PR. The proteolytic maturation of the virions is accompanied by morphological changes. A characteristic feature is the condensation of the inner core, resulting in the formation of a conical core cylinder typical for the mature virus (for a review, see Swanstrom and Wills, 1997).

1.3.4. Ubiquitin/Proteasome Pathway and Retrovirus Replication

Information on the importance of the UPS for particular sections of HIV replication is also known: on the one hand, the system is utilized for proteolysis of de novo synthesized virus receptor CD4. This pathway is mediated by the HIV-1-specific protein Vpu which directs CD4 from the membrane of the endoplasmic reticulum (ER) to the site of proteosomal degradation in the cytoplasm (Schubert et al., 1998). There are furthermore indications that, after the virus has entered, proteasomes degrade Gag proteins and thereby reduce the infectivity of entering HIV particles (Schwartz et al., 1998). Moreover, monoubiquitinated forms of Gag have been described for HIV-1 and Mo-MuLV Gag proteins (Ott et al., 1998).

Although the catalytic activities of the 26S proteasome are completely different from the very specific aspartate-protease activity of the HIV-1/HIV-2 viral proteases, it was observed that a specific inhibitor of the HIV-1 protease, referred to as “ritonavir” (but none of the other previously known HIV protease inhibitors) can inhibit chymotrypsin activity of the 20S proteasome in vitro (Schmidtke et al., 1999) and proteasome-mediated MHC-I antigen expression in vivo (André et al., 1998). Based on this phenomenon which is described in the scientific literature, von Bryant et al. (2000) filed a patent application which is focused on the use of HIV-1 protease inhibitors as proteasome inhibitors in the treatment of cancer, of inflammatory disorders and of non-HIV-related infections (WO 00/33654). The subject matter of the present application is not affected thereby.

2. Biology of Hepatitis Viruses

2.1. Human and Animal Hepatitis B Viruses

In addition to human HBV, a multiplicity of related animal viruses are known which together form the family of “hepadnaviruses” (for a review, see Schäfer et al., 1998). These viruses have in common the synthesis of a pregenomic RNA from a circular supercoiled form of the genome (cccDNA) in the nucleus, cytoplasmic packaging of a pregenomic RNA into nucleocapsids, transcription of said pregenomic RNA inside the capsid into a circular partially double-stranded DNA form (ocDNA) with the aid of the virus-encoded reverse transcriptase with DNA-polymerase activity during virus maturation and export. Due to the numerous features shared with HBV, the animal hepadnaviruses are used very frequently as animal models for researching the biology, pathogenesis and evaluation of antiviral substances for the therapy of human hepatitis B (for a review, see Schäfer et al., 1998). In the present specification, therefore, particular use was made of the duck hepatitis B virus animal model for studies on proteasome inhibitors.

2.1.1. Hepadnaviral Virus Particles and Components

The serum of viremic patients and animals usually contains, in addition to the infectious virion (approx. 42 nm in diameter), 1000 to 10 000 rather subviral, noninfectious particles of spherical or filamentous shape. The viral particles consist of a lipid envelope in which HBV genome-encoded surface proteins (HBS or S, PreS1 or large S, PreS2 or middle S) are embedded. The nucleocapsid consists of the nucleocapsid protein and contains the partially double-stranded viral genome and cellular proteins. These include, inter alia, cellular kinases.

2.1.2. The Early Processes in Hepadnavirus Infections

After binding of the viral particles to surface molecules of hepatocytes and other cells of hepatic and nonhepatic origin, the virus is transported via poorly understood mechanisms into the cells and the viral genome is transported into the nucleus. Each of the processes of the early steps of infection, in which the interaction with cellular components plays a part, could be disrupted in proteasome-treated cells and thus explain the lack of or low infectivity of virions, which was surprisingly found in the present invention. The viral genome, during or after entering the nucleus, is converted to a completely double-stranded supercoiled DNA genome (cccDNA). All viral RNAs are synthesized from said cccDNA. The excessive genome length RNA is terminally redundant and apart from pregenomic RNA, subgenomic RNAs are synthesized from which the structural and nonstructural proteins are translated.

2.1.3. The Late Steps of Hepadnavirus Replication

Phosphorylation and dephosphorylation of the core protein play an important part in the assembly of the nucleocapsid, in DNA synthesis, in the association of the nucleocapsid proteins with the nuclear membrane and their transport into the nucleus, and in nucleocapsid disintegration which is necessary in order to transport the genome into the nucleus. Modifications in the phosphorylation of the nucleocapsid can interfere with the infectivity of the hepadnaviruses and with the infection process. Once DNA synthesis has reached a particular state of maturation, the virus is enveloped. Some of the nucleocapsids migrate to the nuclear membrane and thus provide the necessary increase in the cccDNA copy number.

2.1.4. The Problem of Hepadnavirus Homogeneity

The step of reverse transcription of the HBV genome into DNA and the lack of a proof-reading function in the reverse transcriptase lead frequently to mutations during HBV propagation, in a manner similar to that known for HIV and other RNA viruses, including HCV (for a review, see Günther et al., 1998). For this reason, patients are always infected with a very heterogeneous population of HBV and HCV. Said heterogeneity influences pathogenesis, viral resistance, response to therapy with interferons (IFN) and antiviral substances (nucleoside analogs and others) and detection of the infected cells by the immune system. These findings prove that novel antiviral strategies are necessary and useful for preventing a de novo HBV infection and especially for the therapy of chronic infection, despite the availability of a vaccine and prophylactically administerable antibodies.

2.2. Possible Immunoprotective Therapies of HBV Infection

One of the few possible therapies of chronic HBV infection is the treatment with interferons (IFN). This is an established and approved therapy which is, however, only at least partially effective. In co-infections with HIV and HCV which occur relatively frequently, the success rate of an IFN treatment is even lower than in an infection with HBV or HCV alone. Even a clinically successful IFN treatment practically never eliminates all reservoirs of HBV and HCV which can reactivate the infection (Rehermann et al., 1996). An anti-HBV therapy based on IFN administration has the substantial disadvantage of being frequently associated with negative side effects (for a review, see Trautwein and Manns, 2001).

The nucleoside analogs used for HBV inhibit transcription of the pregenomic RNA into DNA by the virus-encoded polymerase. Nucleoside analogs (e.g. lamivudine, famcyclovir, adevofir and entacavir) have the great disadvantage of almost always causing the selection of medicament-resistant HBV strains. Moreover, nucleoside analogs harbor the risk of possibly causing chromosomal mutations and thus cancer. The novel medicaments mentioned within the scope of the invention presented herein do not carry this risk and said side effects.

2.3. Biology and Treatment of HCV Infections

The infection with hepatitis C virus (HCV) is also one of the great problems of world health. Approximately 170 million people (i.e. approx. 3%) of the world's population are infected with this virus. In some countries, more than 10% of the population are infected with HCV. In contrast to the HBV, there are thus far no effective vaccines for HCV. In approx. 80% of cases, HCV infection is chronic with inflammations of the liver of differing degrees of severity. Similarly to HBV, chronic HCV infection is also associated with a very high risk of developing liver cirrhosis and liver carcinoma. For both diseases, there is hardly any chance of a cure other than a successful liver transplant. HCV belongs to the flaviviruses and encodes approx. 10 gene products. The infection is usually diagnosed by determining the specific anti-HCV antibodies, the viral antigens and RNA. The pathogenesis is similar to that of HBV and is distinguished by differing degrees of inflammation of the liver up to liver failure, the development of liver cirrhosis/fibrosis and of liver carcinoma as well as accompanying disorders. The therapy is, similarly to that of HBV, also based mainly on the treatment with interferon alpha and derivatives and with nucleoside analogs and further substances of unknown action (for a review, see Trautwein and Manns, 2001). The guanosine analog ribavirin has been approved in combination with interferons for the therapy of chronic HCV since 1999. However, the action of this medicament is only incompletely understood. The administration of ribavirin cannot be expected to completely eliminate HCV. Moreover, ribavirin frequently has a number of side effects (for a review, see Trautwein and Manns, 2001). The fact that there are a multiplicity of nonresponders for whom the only possible help is fundamentally new medicaments as will be described in the present invention, applies to all currently approved medicaments for HBV and HCV and also HDV (hepatitis D virus).

2.4. Connection Between the UPS and the Replication Cycle of Hepadnaviruses

One of the regulatory proteins of HBV, the HBx protein, was shown to interact with a subunit of the 26S proteasome complex, said interaction being essential for HBx function (Hu et al., 1999). It was furthermore reported that HBV- and HCV-antigenic determinants can be efficiently presented in the form of MHC class I peptide complexes even in the presence of proteasome inhibitors (López et al., 2000). Thus, a therapy with proteasome inhibitors, as proposed in the present specification, would not influence the recognition of HBV-infected liver cells by cytotoxic T_{CD8+} cells and thus the cellular natural immunity against HBV infection.

2.4.1 Function of the UPS in HCV Replication

The expression of HCV proteins, unlike that of HBV proteins, does not influence the activity of the UPS (Moradpour et al., 2001). These results suggest that MHC class I antigen presentation and the processing of viral antigens are not influenced by HCV and that therefore other immunoescape mechanisms are required for establishing a persistent HCV infection. A fraction of the HCV core protein itself is degraded via UPS, and a monoubiquitinated form of the core protein was observed (Suzuki et al., 2001).

2.4.2. Function of the UPS in HCC Replication

Up until now, it has not been tested whether proteasome inhibitors influence proliferation or the state of transformation of hepatocellular carcinomas (HCC). The only known fact is the overexpression of a proteasome subunit in most HCCs (Higashitsuji et al., 2000).

On the basis of the known prior art, it can be stated that the surprisingly found antiviral action of proteasome inhibitors on late processes of retroviral replication, such as, for example, Gag processing, assembling and budding of HIV-1 or HIV-2 virions, and on the production of infectious virus progeny or on the entire virus replication cycle has not been reported as yet. Likewise, there are no reports on the use of proteasome inhibitors for the treatment of infections with HIV or other retroviruses. Furthermore, it can be concluded that none of the studies previously published in the specialist or patent literature or other work published to date have tested or reported an influence exerted by proteasome inhibitors on the release and infectivity of hepatitis viruses, as will be illustrated in the present description of the invention. In particular, no antiviral effects by proteasome inhibitors on the processes of the HBV replication cycle have been determined. Furthermore, it has not been reported that proteasome inhibitors preferably destroy liver carcinoma cells generated by hepatitis infections and are therefore suitable for the therapy of liver carcinomas. The actions of proteasome inhibitors, illustrated according to the invention, on early and late processes of HBV replication and also on the development of secondary liver cirrhosis and liver carcinomas thus represent entirely novel principles of the antiviral treatment of HBV infections. The use of proteasome inhibitors in the antiviral therapy of hepatitis infections, especially for preventing an acute and chronic HBV and HCV infection from being established or maintained, has not been reported to date.

The following patent applications which do not directly relate to the present invention have been published: an invention which describes compositions for interfering with HBV infections, which are based on the interaction of HBV protein X with proteasome subunits (U.S. Pat. No. 5,872,206); a method for determining the proteasome activity in biological samples (WO 00/23614); the use of proteasome inhibitors as compositions for the treatment of cancer, inflammations and autoimmune diseases (WO 99/22729); the use of inhibitors of the UPS as compositions for the treatment of inflammations and autoimmune diseases (WO 99/15183).

### SUMMARY OF THE INVENTION

The invention is based on the object of providing compositions which are suitable for the treatment of viral infections and which, in particular,

inhibit the release and maturation of infectious retroviruses, especially immunodeficiency viruses such as, for example, HIV-1 and HIV-2, and

can be used for the treatment, therapy and inhibition of a viral hepatitis.

The object was achieved by using at least one proteasome inhibitor. According to the invention, compositions were developed for the treatment of viral infections, whose active components are proteasome inhibitors contained in pharmaceutical preparations. The invention relates to viral infections with, in particular, those viruses which are released from the cell surface during the replication cycle. According to a preferred embodiment of the invention, the proteasome inhibitors used are substances which inhibit, regulate or otherwise influence the activities of the ubiquitin/proteasome pathway.

Possible proteasome inhibitors used are also substances which influence specifically the enzymic activities of the complete 26S proteasome complex and of the free, catalytically active. 20S proteasome structure not assembled with regulatory subunits. Said inhibitors can inhibit either one or more or all three major proteolytic activities of the proteasome (the trypsin-, chymotrypsin- and postglutamyl peptide-hydrolyzing activities) in the 26S or else the 20S proteasome complex.

One variant of the invention comprises using as proteasome inhibitors substances which are absorbed by cells of higher eukaryotes and which, after adsorption into the cell, interact with the catalytic beta subunit of the 26S proteasome and, in the process, irreversibly or reversibly block all or individual proteolytic activities of the proteasome complex.

Another form of the invention uses compositions which inhibit the activities of the ubiquitin-conjugating as well the ubiquitin-hydrolyzing enzymes. They also include cellular factors interacting with ubiquitin in the form of mono- or else polyubiquitin. Polyubiquitination is generally regarded as a recognition signal for proteolysis by the 26S proteasome, and influencing the ubiquitin pathway can likewise regulate proteasome activity.

Proteasome inhibitors used according to the invention are also substances which are administered in vivo in various forms orally, intravenously, intramuscularly, subcutaneously, in encapsulated form with or without cell specificity-carrying modifications or otherwise, which have, owing to application of a particular administration regime and dose regime, low cytotoxicity and/or high selectivity for particular cells and organs, cause no or negligible side effects, have a relatively long metabolic half-life and a relatively slow clearance rate in the organism.

Other proteasome inhibitors used are substances which are isolated in their natural form from microorganisms or other natural sources, are derived from natural substances by chemical modifications or are totally synthesized or synthesized in vivo by means of gene therapy methods or prepared in vitro or in microorganisms by means of genetic methods. They include:

a) naturally occurring proteasome inhibitors:

epoxomicin (epoxomycin) and eponemycin,

aclacinomycin A (also referred to as aclarubicin),

lactacystin and chemically modified variants thereof, in particular the cell membrane-penetrating variant “Clastolactacystin β-lactone”,

b) synthetically prepared:

modified peptide aldehydes such as, for example, N-carbobenzoxy-L-leucinyl-L-leucinyl-L-leucinal (also referred to as MG132 or zLLL), its boric acid derivative MG232; N-carbobenzoxy-Leu-Leu-Nva-H (referred to as MG115); N-acetyl-L-leucinyl-L-leucinyl-L-norleucinal (also referred to as LLnL); N-carbobenz-oxy-Ile-Glu(Obut)-Ala-Leu-H (also referred to as PSI);

peptides carrying C-terminally α,β-epoxyketones (also referred to as epoxomicin/epoxomycin or eponemycin), vinyl sulfones (for example carbobenzoxy-L-leucinyl-L-leucinyl-L-leucine vinyl sulfone or 4-hydroxy-5-iodo-3-nitrophenylactetyl-L-leucinyl-L-leucinyl-L-leucine vinyl sulfone, also referred to as NLVS), glyoxal or boric acid radicals (for example pyrazyl-CONH(CHPhe)CONH(CHisobutyl)B(OH)₂), also referred to as “PS-431” or benzoyl(Bz)-Phe-boroLeu, phenacetyl-Leu-Leu-boroLeu, Cbz-Phe-bbroLeu); pinacol esters, for example benzyloxycarbonyl(Cbz)-Leu-Leu-boroLeu-pinacol ester; and

particularly suitable compounds used are peptides and peptide derivatives carrying C-terminally epoxyketone structures, which include, for example, epoxomicin (empirical formula: C₂₈H₈₆N₄O₇) and eponemycin (empirical formula: C₂₀H₃₆N₂O₅)

chemically modified derivatives based on naturally occurring compounds, in particular a β-lactone derivative referred to as PS-519 (1R-[1S,4R,5S]]-1-(1-hydroxy-2-methylpropyl)-4-propyl-6-oxa-2-azabicyclo-[3.2.0]heptane-3,7-dione, empirical formula: C₁₂H₁₉NO₄) which is derived from the natural proteasome inhibitor lactacystin;

particular dipeptidyl-boric acid derivatives, in particular compounds derived from the pyranocyl-phenyl-leucinyl-boric acid derivatives denoted “PS-341” (N-pyrazinecarbonyl-L-phenylalanine-L-leucine-boric acid, empirical formula: C₁₉H₂₅BN₄O₄). They furthermore include the compounds “PS-273” (morpholino-CONH—(CH-naphthyl)-CONH—(CH-isobutyl)-B(OH)₂) and its enantiomer PS-293, the compound PS-296 (8-quinolylsulfonyl-CONH—(CH-naphthyl)-CONH(—CH-isobutyl)-B(OH)₂); the compound PS-303 (NH₂(CH-naphthyl)-CONH—(CH-isobutyl)-B(OH)₂); the compound PS-321 (morpholino-CONH—(CH-naphthyl)-CONH—(CH-phenylalanine)-B(OH)₂); the compound PS-334 (CH₃—NH—(CH-naphthyl-CONH—(CH-isobutyl)-B(OH)₂); the compound PS-325 (2-quinol-CONH—(CH-homo-phenylalanine)-CONH—(CH-isobutyl)-B(OH)₂); the compound PS-352 (phenylalanine-CH₂—CH₂—CONH—(CH-phenylalanine)-CONH—(CH-isobutyl)-B(OH)₂); the compound PS-383 (pyridyl-CONH—(CHpF-phenylalanine)-CONH—(CH-isobutyl)-B(OH)₂. All these compounds have already been described, inter alia in Adams et al. (1999).

Apart from epoxomicin and eponemycin, the proteasome inhibitors, PS-519, PS-341 and PS-273 (developed by Millennium Pharmaceuticals Inc., Cambridge, Mass. 02139) have proved to be particularly suitable compounds. These proteasome inhibitors are very potent, very proteasome-specific, do not block any, other cellular proteases and therefore have practically no side effects. Moreover, the proteasome inhibitors PS-341 and PS-519 have been tested both in animal models for preclinical studies and in humans (cancer patients) for clinical studies.

The proteasome inhibitors provided according to the invention are compositions which surprisingly

impair, by blocking late processes in retroviral replication, the production of infectious progeny viruses and thus prevent the infection from spreading in the organism;

block the release of infectious hepatitis viruses, in particular of HBV and HCV, from infected cells;

restrict the spread of an acute infection with hepatitis viruses;

cause the preferred death of liver carcinoma cells;

suppress viremia both in a new infection and in chronic infections with hepatitis viruses and increase the success of a virus elimination by the endogenous immune system and/or by known compositions with a similar or different action.

Surprisingly, we found within the scope of the present invention that proteasome inhibitors inhibit late processes in the replication cycle of retroviruses. Specifically, we found that the use according to the invention of proteasome inhibitors is suitable for preventing the assembly and release of virions from the cell surface. This entails the inhibition of proteolytic processing of Gag structural proteins by the viral protease. Likewise, the infectivity of the released virions is reduced.

It is possible to inhibit the following retroviruses: spumaviruses, mammalian C-type oncoviruses, BLV (bovine leukemia virus), HTLV (human T-cell leukemia virus), leukemia viruses, RSV (Rous sarcoma virus) viruses or lentiviruses. Suitable examples of leukemia viruses are BLV, HTLV-I or HTLV-II, examples of lentiviruses are human immunodeficiency virus type 1 (HIV-1), human immunodeficiency virus type 2 (HIV-2), simian immunodeficiency virus (SIV), feline immunodeficiency virus (FIV) or bovine immunodeficiency virus (BIV).

The invention relates, likewise by using proteasome inhibitors, to the control/treatment of disorders/pathological symptoms caused by infections with retroviruses. Said disorders/pathological symptoms may be caused by infections with leukemia viruses, human T-cell leukemia viruses HTLV-I and HTLV-II or by infections with lentiviruses.

Another field of application of the invention is the control/treatment of AIDS by means of proteasome inhibitors, both in the early symptom-free and in the advanced phases of the disease. Said proteasome inhibitors may also be used in combination with other antiretroviral medicaments, for example with blockers of viral RT and PR. The combination with antiretroviral therapies based on gene therapy interventions is also possible. Another use results from the combination with intracellular immunization such as, for example, introducing genes having anti-HIV-1/HIV-2 activity into stem cells and/or peripheral CD4⁺ lymphocytes.

According to the invention, it is likewise possible to prevent the onset of the disease and to reduce the spread of the infection in the organism (reduction of viral load) of symptom-free HIV-1/HIV-2 seropositive and HIV-1/HIV-2-infected individuals. Furthermore, it is possible to use proteasome inhibitors for treating/controlling/preventing HIV-induced dementia, in particular for preventing HIV-infection of neurons, glia and endothelial cells in capillaries of the brain. Another use is to prevent establishment of a systemic HIV-1/HIV-2 infection immediately after contact with infectious virus (for example in the case of needle puncture injuries with HIV-contaminated blood or blood products).

It is likewise possible to achieve the use of proteasome inhibitors in basic research for understanding retrovirus assembly, the action of the viral proteases, in studies on Gag processing, in the development of further substances influencing Gag processing, in studies for understanding cellular mechanisms involved in late processes of retrovirus assembly.

The object is achieved in principle as demonstrated, by way of example, for HIV-1 and HIV-2. The inhibition of the production of infectious virus particles immediately after addition of various substance classes of proteasome inhibitors is demonstrated. According to the invention, this phenomenon is observed both in HIV-1-infected permanent cultures of CD4⁺ human T cells and in human fibroblast (HeLa cell) cultures transfected with infectious proviral DNA of HIV-1 and HIV-2 and is described in more detail herein. On the basis of said novel activities of proteasome inhibitors, it can be assumed that administration of proteasome inhibitors tolerated in vivo can suppress or completely eliminate the spread of infection with HIV in the organism. According to the invention, it is demonstrated that the inhibitory effect of proteasome inhibitors on HIV replication includes the following mechanisms:

1) blocking/reducing proteolytic processing of the Gag polyproteins by HIV-1 PR (protease);

2) blocking/reducing the release and budding of new virions on the cell membrane;

3) blocking/reducing the infectivity of released virions;

4) blocking/reducing the spread of the infection with HIV-1 in cultured CD4⁺ T cells.

These mechanisms may be associated directly or indirectly with the phenomenon that ubiquitin ligases interact with L (late assembly) domains of HIV-1 Gag proteins (Strack et al., 2000, category XXX) and that, after inactivation of the UPS, monoubiquitination of HIV-1 Gag proteins containing L domains is blocked (Schubert et al., 2000 and Ott et al, 2000, category XXX).

The object was achieved by carrying out, within the scope of the invention, various protein-chemical, molecular-virological and morphological studies on HIV-1. According to the, invention, the defect in Gag processing caused by proteasome inhibitors is illustrated by means of biochemical methods. For this purpose, metabolic pulse labeling of HIV-proteins by means of radioactive amino acids, followed by an incubation (chase) in nonradioactive medium, was carried out. The information obtained makes it possible to illustrate the inhibitory effect of proteasome inhibitors on Gag processing and budding of HIV virions within short time-frame kinetics which correspond to part sections of an HIV replication cycle.

It is demonstrated, according to the invention, that the inhibitory action of proteasome inhibitors on HIV assembly and release does not affect the enzymic activity of HIV-1 PR. In vitro processing studies on isolated HIV-1 Gag and PR molecules demonstrate that various substance classes of proteasome inhibitors exert no influence whatsoever on the enzymic activity of PR.

The invention further illustrates the reduced infectivity of released immature HIV virions, due to the action of the proteasome inhibitors, by means of end point titration studies in CD4⁺ T-cell cultures. It is demonstrated here that incubation with proteasome inhibitors for just 6 hours (corresponds to approximately a third of an HIV replication cycle in the target cell) leads to a 10-fold reduction in the virus titer and to a 50-fold reduction in the specific infectivity of the released virus particle.

According to the invention, the influence of proteasome inhibitors on the morphology of HIV-1 virions in the assembly and budding process on the cell membrane is studied. This object is achieved by carrying out high-resolution transmission electron microscopy on HIV-1-infected CD4⁺ T cells. It is found that the treatment with proteasome inhibitors for a period of approximately 5 hours leads to the following changes in the morphology of the virus:

1) the arrest of assembling virions in the budding phase is substantially increased;

2) the detachment of the virions from the cell surface is disrupted, leading to the formation of virus-membrane connections (stalk formation);

3) the absolute number of virus particles on the cell surface is reduced;

4) the relative number of immature cell-free virions is increased.

The invention demonstrates the inhibitory effect of proteasome inhibitors on virus replication in cultures of HIV-1-infected CD4⁺ T cells. The addition of nanoM concentrations of various substance classes of proteasome inhibitors prevents the infection from spreading and causes the absence of a productive virus replication.

The principle of using proteasome inhibitors for blocking an infection with HIV, illustrated in the description of the invention, is novel with regard to the use of an already known substance class (proteasome inhibitors) for a new activity (blockage of Gag processing and release of retroviruses).

At the same time, the use of proteasome inhibitors is also novel with regard to the administration principle. Up until now, no substances/principles/methods were known which influence late processes of HIV replication, without requiring mutations in the virus itself.

Furthermore, the fact that using proteasome inhibitors for blocking HIV and other retroviruses does not affect the virus itself but mechanisms which are conserved in all host cells of said virus is also novel. In comparison with previous antiretroviral methods which relate to essential components of the virus per se, the probability of the development of resistance mechanisms is several orders of magnitude lower in the case of administering proteasome inhibitors in antiretroviral therapy. The novelty of this principle of action of proteasome inhibitors is also demonstrated by the fact that proteasome inhibitors have a broad spectrum of action with respect to different isolates of HIV-1 and HIV-2. Within the scope of the invention, the same intensity of the inhibitory effect was observed for various primary and also cell culture-adapted T cell-tropic and macrophage-tropic HIV isolates.

Furthermore, the principle of the action of proteasome inhibitors which prevent the production of infectious virus particles by already infected cells, if not the virus from entering, is also novel. This should make it possible to reduce substantially the amount of infectious virions (viral load) and thus the spread of the infection in vivo. The average survival time of an acutely HIV-infected T cell is a few days. Moreover, the inhibition of virus release and the accumulation associated therewith of partly toxic HIV proteins (in particular of the Env envelope proteins) result in an increased cytopathic effect and thereby to a faster death of the infected cell. Apart from de novo infection, the action of proteasome inhibitors should also lead to a faster death of already infected cells.

In summary of these novel mechanisms, it can be concluded that, in the case of an in vivo administration of proteasome inhibitors, the reduced release of virus particles which have, in addition, low, if any, infectivity with simultaneous cell death of the virus-producing cells in a net effect is reduced the amount of infectious virions in peripheral blood and, at the same time, the number of infected HIV-producer cells in the whole organism. This renders attractive the administration of proteasome inhibitors alone or in combination with enzyme inhibitors already used in antiretroviral therapy.

Proteasome Inhibitors Reduce the Infectivity of HIV-1 Virus Particles

The present invention demonstrates, for the first time, that, immediately after inactivation of the proteasome pathway by treatment of HIV-1-infected T cells, the release of virus particles is reduced. Furthermore, the specific infectivity of the released virions is reduced, thereby reducing dramatically the specific titer of newly produced virus particles (for this, see exemplary embodiment 1).

To this end, according to the invention, CD4⁺ T cells are infected with HIV-1 and, at the time of maximum virus production (approx. 80% of the culture are in the acute infection phase), treated with proteasome inhibitors for various periods, up to a maximum of 4.5 hours. At various times after the treatment, virus-containing cell culture supernatants are harvested. The amount of released virions is determined by means of anti-capsid antigen ELISA, and the specific infectivity of the newly produced virions is determined by means of end point titration.

According to the invention, this result indicates a novel activity of proteasome inhibitors. This activity cannot be attributed to purely unspecific impairment of cell metabolism due to switching off the UPS, namely for the following reasons:

This effect is active very early on, at a time when it is not yet possible for potential negative effects of proteasome inhibition on cellular processes such as, for example, protein synthesis and protein folding and the functions of chaperones to be active.

The released virions have a markedly reduced infectivity which is explained, in the further description of the invention, by a defect in the proteolytic maturation of HIV particles, which is caused specifically by proteasome inhibitors.

Toxic effects such as unspecific cell lysis, apoptosis or unspecific release of cellular and viral proteins were not detected under the experimental conditions described here of the treatment with proteasome inhibitors.

The inhibitory action of proteasome inhibitors can be attributed exclusively to the influence of cellular processes required for virus assembly, release and viral maturation, but not to chemical modifications of the released virions themselves. This fact is reflected by the observation made according to the invention that the infectivity of cell-free HIV-1 virions produced in cells with active proteasome pathway was not impaired when said virions were treated with relatively high concentrations of proteasome inhibitors prior to titration.

In summary, it can be concluded herewith that proteasome inhibitors disrupt the production of infectious HIV-1 particles by influencing cellular processes. The mechanism of this effect is illustrated in more detail below.

Electron-Microscopic Analysis of HIV-1-Infected Cells

Within the scope of the present invention, the specific action of proteasome inhibitors on assembly and budding of HIV-1 virions is studied with the aid of transmission electron microscopy (for this, see exemplary embodiment 2). According to the invention, this method elucidates the novel action of proteasome inhibitors on the morphology of the virus. To this end, acutely infected T cells are treated with proteasome inhibitors for 5 hours. The cells are enclosed in cellulose capillaries, incubated, fixed and used for thin sections. This method has the substantial advantage of virions being retained in the cellulose capillaries and, as a result, there is no need for concentration and thus modification of the morphology of the virus by centrifugation.

According to the invention, essential phenomena of the action of proteasome inhibitors on the morphology of the virus are observed in this study:

a relative reduction in the number of extracellular virus particles and budding structures on the cell membrane;

a relative increase in the number of immature virus particles which remain attached to the cell membrane and cannot detach completely.

These electron-microscopic studies confirm the biochemical and virological observations made within the framework of the invention that the treatment with proteasome inhibitors reduces the release and infectivity of new virions. At the same time, said electron-microscopic studies indicate that the treatment with proteasome inhibitors does not adversely affect proteolytic maturation and thus the maturation of virus particles. The essential process of HIV maturation is the proteolytic cleavage of the Gag polyproteins by the viral protease. The further studies show that exactly this process of proteolytic maturation is inhibited by proteasome inhibitors.

Proteasome Inhibitors Reduce the Kinetics of Gag Processing and Virus Budding of HIV-1 and HIV-2

Within the framework of the present invention, it is detected by means of pulse/chase analyses that proteasome inhibitors substantially reduce the kinetics of Gag processing and virus release/budding (for this, see exemplary embodiment 3). To this end, according to the invention, cells (either T cells infected with HIV-1 or HeLa cells transfected with infectious proviral DNA of HIV-1 or HIV-2) are labeled, at the time of maximum expression of viral proteins, metabolically in the cell -with [³⁵S]-methionine for a relatively short pulse period of approx. 30 min. Subsequently, the cells are incubated in the absence of radiolabeled amino acids. At various times during the chase period, equivalent samples are obtained from the cells, the medium and the virions isolated by centrifugation. Transport, processing and assembly of the radiolabeled viral proteins are monitored over a total chase period of 8 hours. The individual HIV proteins are isolated by immunoprecipitation by means of HIV-specific antibodies and AIDS patients' sera from the intracellular, the extracellular and the virus-associated fractions, fractionated in SDS-PAGE and then quantified by image analysis. For a particular experiment, parallel cultures cells are treated with proteasome inhibitors, while the cells remain untreated in the control culture. In order to block proteasome activity as completely as possible, the cells are treated with in each case 25 microM zLLL and LC. Approx. 30 min. before the start of pulse labeling, the inhibitors are added to the cell culture medium. This short preincubation neither impairs protein biosynthesis nor causes any substantial changes in the concentration of cellular chaperones (Schubert et al., 2000, category XXX). The relative amount of labeled Gag polyprotein Pr55 and of the main processing product p24 capsid (CA) is determined for each time of sample removal in the cell, medium and virus fractions. Based thereupon, the rate of virus release (which corresponds to the amount of radiolabeled Gag appearing in the virus fraction within 8 hours after synthesis) and Gag processing (rate of conversion of radiolabeled Gag Pr55 to CA) is determined.

According to the invention, an approx. 6-fold reduction in the kinetics of virus release within the first two hours after pulse labeling is observed in cells with inactivated proteasomes (FIG. 2). Parallel thereto, a similarly large reduction in the kinetics of Gag processing which was determined as the quotient of CA and Pr55 for each chase time was also observed (FIG. 2). This processing effect appears to influence several steps in the maturation of Pr55. The complete cleavage of HIV-1 Pr55 generally produces the mature Gag proteins MA, CA, NC and P6^{gag} and two smaller spacer peptides which link the individual domains to one another. A plurality of these cleavage processes seem to be inhibited in the process of Gag processing, due to the action of proteasome inhibitors, since the appearance of Gag-processing intermediates such as, for example, MA-CA (p41), p39 (Ca—NC) or CA with a 14-amino acid spacer (p25^{CA}) is observed, after blockage of proteasome activity. In contrast to Gag processing, the proteasome inhibitors do not influence processing of the Env envelope proteins, nor do they impair the expression and stability of other viral proteins.

In summary, it is concluded that proteasome inhibitors block Gag processing and the release of virus particles. The degree of inhibition depends on the time of preincubation with proteasome inhibitors prior to the start of the pulse/chase experiment. After approx. 5 hours of preincubation, Gag protein processing and the release of virus particles are nearly completely blocked.

This novel effect of proteasome inhibitors seems to influence a general mechanism of the assembly, budding and maturation of retroviruses. Said effect is not limited specifically to a particular HIV-1 isolate. Comparative analyses of macrophage-tropic HIV-1 isolates show similar effects as those observed for T cell-tropic HIV-1 isolates. In accordance with the invention, an inhibitory effect of proteasome inhibitors on Gag processing and virus release of various HIV-2 isolates is also observed (FIG. 2, Panel HIV-2 in HeLa).

It is furthermore demonstrated that the phenomena described within the framework of the invention need a specific inhibition of the 26S proteasome complex. Testing of peptide aldehydes which are similar to zLLL cytosolic caspases, but do not inhibit the proteasome, do not exert any inhibitory action on HIV-1 Gag processing and virus release.

On the basis of the novel activity of proteasome inhibitors, it can thus be concluded that

proteasome inhibitors block both the processing of Gag proteins and the release and budding of new virions from the cell surface;

this inhibitory property of proteasome inhibitors is not limited to individual isolates of HIV-1 but applies also to other lentiviruses such as, for example, HIV-2;

said novel activity is not based primarily on unspecifically acting negative effects of proteasome inhibitors on cellular and viral processes, since this effect is active selectively for Gag processing and virus release in the process of virus assembly and maturation;

this phenomenon requires selective inhibition of the activity of the 26S proteasome complex.

Since Gag processing, together with an efficient release of virus particles, is an essential component of the retroviral replication cycle and thus essential for the production of infectious virions, it can be assumed that the novel function described herein of proteasome inhibitors is suitable for suppressing the spread of infection with HIV-1 and HIV-2 (the two pathogens of the AIDS pandemic) in vivo. Since HIV-infected cells in general have only a limited lifetime and since, in addition, blocking of virus release even accelerates the death of infected cells, said novel activity of proteasome inhibitors can, in the case of in vivo administration, both prevent de novo infection of noninfected cells and induce the death of already infected cells and thus completely eradicate (wipe out) the infection. In the case of HIV-1, efficient Gag processing is also known to be a precondition for efficient virus release. The two novel effects of proteasome inhibitors on late processes of viral replication are thus directly linked to one another and can therefore amplify but not eliminate each other.

Proteasome Inhibitors do not Influence the Enzymatic Activity of the HIV-1 Protease

In order to understand the mechanism of the novel effects shown in the framework of the invention, it is essential to understand in more detail the specific action of proteasome inhibitors on the viral protease of HIV. The simplest explanation of the novel phenomena described in the present invention would be direct inhibition of said HIV protease by proteasome inhibitors. This connection, however, seems unlikely, since both protease complexes operate with completely different enzymic mechanisms: while the HIV-1 protease is active as a dimer and operates according to the mechanism of aspartate proteases, the proteasome is a multienzyme complex having several active sites. Despite the fact that the proteolytic activities and catalytic mechanisms of HIV proteases and of the proteasome complex are fundamentally different, influencing the viral protease by proteasome inhibitors seems at least theoretically possible, since it has been reported in the literature that the HIV-1-specific protease inhibitor ritonavir inhibits the chymotrypsin activity of the 20S proteasome (André et al., 1998; Schmidtke et al., 1999).

Therefore, a possible influence of proteasome inhibitors on the enzymic activity of the HIV-1 protease is investigated within the framework of the invention by studying the in vitro processing of Gag polyprotein (for this, see exemplary embodiment 4). For this purpose, according to the invention, recombinant Pr55 is expressed in insect cells and purified from released virus-like particles. Enzymatically active HIV-1 protease is expressed in E. coli and chromatographically purified. The specific activity of the protease is determined by means of titration of the active sites. Pr55 and protease are set to an enzyme-to-substrate ratio of 1:25 and incubated for a defined time under conditions under which only approx. 50% of the substrate Pr55 is proteolytically processed. These reaction conditions allow sensitive detection of small effects on the enzymic activity of the protease. Even under these sensitive reaction conditions and with a very high inhibitor concentration of up to 100 microM, no inhibitory action whatsoever of both protease inhibitors, LC and zLLL, on Gag processing is detected (FIG. 3).

In summary, the results obtained within the framework of the invention indicate that proteasome inhibitors do not influence the enzymic activity of the HIV protease and that therefore the viral protease cannot be the direct target of said inhibitors. The investigation of the basic mechanism allows the conclusion that cellular processes which are essential for Gag processing and virus release of HIV are the target of the negative action of proteasome inhibitors. On the basis of the novel observation described for the first time within the scope of the invention, it can thus be concluded that, in contrast to antiretroviral therapies already used (inhibitors of the viral enzymes protease and reverse transcriptase), no viral factor is the direct target of this novel action. Owing to the fact that a cellular mechanism is the target of the inhibitors, the risk of HIV developing resistance to proteasome inhibitors in the case of in vivo adminstration of proteasome inhibitors to suppress HIV replication in HIV-infected subjects would be comparatively low or nonexistent.

Proteasome Inhibitors Inhibit HIV-1 Virus Replication in Cell Culture

After it had been demonstrated, according to the invention, that proteasome inhibitors inhibit late processes of virus release and Gag processing in the retroviral replication cycle, it was important to demonstrate this negative effect also for the spread of the infection and thus for the complete HIV replication cycle. On the basis of the phenomena observed within the framework of the invention, namely that proteasome inactivation causes a reduced release of virions with reduced infectivity, it can be assumed that treatment with proteasome inhibitors likewise results in a reduced spread of the infection in an HIV-infected culture.

For this purpose, according to the invention, parallel cultures of a CD4⁺ T-cell line (for example A3.01) are infected with a defined infectious dose of HIV-1. After infection, the cells are treated in medium with or without the proteasome inhibitor zLLL at an average concentration of 5 microM for the duration of the culture of approx. 2 weeks (for this, see exemplary embodiment 5). The production of new virions is determined by measuring the accumulation of virus-associated reverse transcriptase activity in the cell culture supernatant.

According to the invention, it is found that, in the presence of zLLL, either only a very reduced productive infection (FIG. 4, panel 2, experiment in A3.01) or no productive infection at all (FIG. 4, panel 1, experiment in A3.01) can be established in the culture. The termination of RT accumulation after approx. one week of incubation in cultures with active virus replication can be attributed to the generally known fact that, from the time of maximum virus replication onward, the number of syncitia formations increases, resulting in HIV-induced cell death being higher than the rate of cell division and, as a consequence thereof, the entire culture stopping.

In order to investigate the efficacy of different concentrations of various proteasome inhibitors with respect to blockage of virus replication, parallel cultures of infected cells are treated, according to the invention, with various concentrations of zLLL (FIG. 4, panel 3, experiment in A3.01) and epoxomicin (FIG. 4, panel 4, experiment in A3.01). In this case, a dose-dependent effect on HIV-1. replication is observed: while 100 nanoM zLLL exert a relatively weak inhibitory effect, 1 microM zLLL completely inhibits replication, as already previously observed for 5 microM zLLL. Compared to zLLL, the very specific proteasome inhibitor epoxomicin is much more potent in its action on proteasome activity. This is reflected in the observation, obtained according to the invention, that 100 nanoM epoxomicin cause a complete and 10 nanoM epoxomicin cause an almost complete blockage of HIV-1 replication (FIG. 4, panel 4, experiment in A3.01). In terms of mechanism, blocking of virus replication in cell cultures can be explained as result of the novel activities of proteasome inhibitors, described within the scope of the invention, as follows:

in the presence of said proteasome inhibitors, fewer virions are released,

the few virions released have a low, if any, infectivity.

This restricts de hovo infection of cells in the culture, and, as a result, the spread of the infection is reduced or stopped completely.

The essence of the invention is the use of known compositions for a new purpose and a combination of known elements, the proteasome inhibitors, and a new action, their use for influencing retroviruses and hepadnaviruses, which, in the form of their new overall action, result in an advantage and the desired success which is the availability now of compositions for inhibiting the release and maturation of retroviruses and of compositions for treatment, therapy and inhibition of viral hepatitis.

The invention is further directed toward the use of proteasome inhibitors for preparing compositions for inhibiting the release, maturation and replication of retroviruses. This includes their use for preparing medicaments for the treatment and prophylaxis of AIDS and of pathological symptoms related thereto of an HIV infection, such as, for example, HIV-induced dementia, HIV-induced disruptions in lipid metabolism, especially the HLS syndrome (HIV-associated lipodystrophy syndrome) and of HIV-induced disruptions of kidney functions, especially the HIVAN syndrome (HIV associated nephropathy).

In another part, the second main focus of the invention, it is surprisingly found that, similarly to the effect on retroviruses, proteasome inhibitors inhibit late processes in the replication cycle of hepadnaviruses. In this connection, it was specifically observed that the inventive use of proteasome inhibitors is suitable for preventing substantially or completely the production of infectious virions from chronically HBV-infected cells. Treatment of HBV-producing cells with proteasome inhibitors entails both inhibition of the release of virions and a virtually complete reduction in the infectivity of the released virions. As a consequence of these novel activities, proteasome inhibitors can suppress virus replication and thus de novo infection of hepatocytes and thus the spread of an HBV infection in vivo in the liver tissue of an HBV-infected individual.

It was likewise found that treatment of chronically HBV-infected liver carcinoma cells with proteasome inhibitors preferably induces the death (mainly by inducing apoptosis) of these cancer cells, while healthy, primary hepatocytes and the other nonproliferating liver cells are much more resistant to a treatment with proteasome inhibitors. Liver carcinomas can hardly be treated with medicaments and are, without liver transplant or liver resection, usually fatal. Proteasome inhibitors therefore gain further therapeutic potential for the treatment of hepatitis virus infections: treatment with proteasome inhibitors can suppress or prevent not only the spread of the infection (by blocking the, production of infectious virions) but also the development, of liver cell carcinomas which is linked to the infection or cure an already established liver cell carcinoma. This claim is based on the fact that treatment with proteasome inhibitors, similarly to the already known antineoplastic action of proteasome inhibitors on a multiplicity of tumors, can cause a specific elimination of liver carcinoma cells in vivo. The antineoplastic action of proteasome inhibitors has not been previously demonstrated for liver cell carcinomas and is therefore a novel therapeutic principle. Proteasome inhibitors may thus be used for treating/controlling/preventing HBV-induced liver cirrhosis, in particular primary liver cell carcinomas. Furthermore, using the novel antiviral action, proteasome inhibitors may be used for the treatment of the following symptomatic and symptom-less hepatitis viral infections: hepatitis A virus (HAV), hepatitis C virus (HCV), hepatitis delta virus (HDV), hepatitis E virus (HEV), hepatitis F virus (HFV), hepatitis G virus (HGV). The treatment of hepatitis B and C with proteasome inhibitors is of particular importance, due to the widespread occurrence, the particularly high pathogenicity and due to association of the chronic infection with the development of liver carcinoma.

The proteasome inhibitors may also be used in combination with other anti-hepatitis medicaments and other therapy plans, for example interferon alpha/beta/gamma and variants thereof (for example pegylated interferons), interleukins, nucleoside analogs (lamivudine, cidovir, ribavirin and others), steroids, plasma exchange, thymosin alpha 1, vaccines, passive and active vaccination, therapeutic and prophylactic vaccination, glycryrrhizin, stem-cell transplants, organ transplants, diet therapy, immunosuppressants, cyclosporins and derivatives thereof, amanditin and derivatives, interleukins and other cytokines, non-proteasome-selective protease inhibitors, azathoprin, hemodialysis, and highly active antiretroviral therapy (HAART) for co-infections of HBV with human immunodeficiency viruses (HIV). Since proteasome inhibitors also exhibit antiviral action on HIV, a treatment of HBV/HIV coinfections, in particular in combination with a HAART therapy, is a main focus of application of the invention.

According to the invention, using proteasome inhibitors makes it likewise possible to prevent the onset of the disease and to reduce the spread of the infection in the organism of symptom-free HBV-infected individuals.

Another use of proteasome inhibitors is prevention of the establishment of a systemic hepatitis virus infection immediately after contact with infectious virus (for example in the case of needle puncture injuries with virus-contaminated blood or blood products).

Another use of proteasome inhibitors is prevention of a hepatitis virus infection in individuals at high risk of a new infection, such as, for example, doctors, high-risk personnel in buildings with large numbers of visitors, drug addicts, travelers in regions in which hepatitis viruses are highly endemic, in the treatment of patients, for relatives of chronic virus carriers. Another use of proteasome inhibitors is prevention of reinfection with HBV in the case of liver and other organ transplants and in the case of cell therapies by administering the compositions prior to, during and after transplantation. The administration of said compositions is indicated both for the high-risk situation when transplanting virus-free organs to chronic virus carriers who continuously have residual viruses which can infect the new organs, and for transferring virus-containing organs from donors to virus-free patients.

The object is achieved in principle as demonstrated, by way of example, for DHBV. The inhibition of the production of infectious virus particles from already infected cells after addition of various substance classes of proteasome inhibitors is illustrated.

Proteasome Inhibitors Block the Release of Infectious Virus Particles from Chronically DHBV-Infected Cells

According to the invention, this phenomenon is illustrated by way of the example of the non-infectibility of primary hepatocytes (duck, marmot, tree shrew, and human), of bile-duct cells, of mixed cultures of hepatocytes and nonhepatocytes, of cells of the hematopoietic system, as well as of established hepatoma cells with hepatitis B, C and D viruses from proteasome inhibitor-treated cells. In addition, this is illustrated by means of analogous infection experiments in animal models in vitro (uPA/RAG2 mouse model, repopulated with liver cells from humans, animals and tree shrews; with ducks, marmots and tree shrews). The procedure is to harvest HBV, HCV, HDV virus particles and combinations thereof from the media of producer cells which have been treated with different doses of proteasome inhibitors for different periods of time and to test the infectivity of said virus particles by infection of hepatitis virus-free cells. To this end, the abovementioned cells are incubated with the virus particles, and then the infection or the absence of infection is checked by analyzing the intra- and extracellular components of progeny viruses. Specifically, the state of de novo synthesis of viral antigens such as, for example, surface proteins and nucleocapsid proteins, is tested by means of immunoblot, ELISA and metabolic labeling. The viral nucleic acids are likewise analyzed (RNA by Northern, DNA by Southern blots and PCR and RT-PCR analyses). Moreover, the viral antigens are detected microscopically by indirect immunofluorescence staining with the aid of virus-specific antibodies.

According to the invention, it is demonstrated by way of of example of chronically DHBV-infected duck hepatocytes that, after treatment with proteasome inhibitors, the viability of the primary hepatocytes is not substantially impaired and, likewise, protein synthesis in these primary cells is not substantially blocked. However, it is found within the framework of the description of the invention that the released virions are in fact noninfectious and therefore cannot cause any de novo infection in primary hepatocytes. The result of the invention thus demonstrates that inhibition of the proteasome pathway by means of proteasome inhibitors blocks the production of infectious DHB virions, without substantially impairing the protein synthesis of liver cells.

The principle solution is demonstrated by way of the example of primary duck hepatocytes which are infected with DHBV preparations.

Chronically DHBV-infected primary duck hepatocytes obtained by collagenase treatment from the liver of duck embryos (hatched from commercially available duck eggs) which have frequently already been infected with DHBV are treated with various classes of proteasome inhibitors, including those proteasome inhibitors which are already used in clinical trials for the treatment of cancer patients (for example the proteasome inhibitors PS-341, PS-273, PS-519). After treatment with proteasome inhibitors, it is found within the framework of the invention that the treated hepatocytes have lost, to a large extent or completely, their ability to produce infectious virus particles. On the basis of these novel activities of proteasome inhibitors, it can be assumed that administration of proteasome inhibitors tolerated in vivo suppresses the spread of the infection with hepadnaviruses in the infected organism. Furthermore, it can be assumed within the scope of the present invention that it is possible, using the novel antiviral strategy, to completely eliminate the virus reservoir in a hepadnavirus infection and thus to partially or completely cure a viral hepatitis. This claim is based in particular on the fact that by using this novel treatment it is possible to prevent the spread of the infection and thus de novo infection of liver cells. Even if the amount of intracellular synthesis of viral proteins and nucleic acids in already infected cells is not detectably reduced, the treatment can result in the complete elimination of the virus, since hepatitis-infected cells usually have only a limited lifetime. The regeneration of liver cells is particularly high in patients with inflammation of the liver. It should therefore not be possible for the initially virus-free hepatocytes which are relatively frequently generated in said patients to be infected de novo, due to treatment with proteasome inhibitors.

According to the invention, it is demonstrated that the inhibitory effect of proteasome inhibitors on virus propagation of hepadnaviruses includes the following mechanisms:

1) blocking/reducing the release of new virions;

2) blocking/reducing the infectivity of released virions;

3) blocking/reducing the spread of infection in a primary hepatocyte culture;

4) blocking/reducing the spread of infection in the liver in vivo.

The object was achieved within the scope of the invention by carrying out various protein-chemical, molecular-virological and immunohistological studies on HBV-infected cells. According to the invention, the defect in the infectivity of hepadnaviruses, caused by proteasome inhibitors, is illustrated by means of biochemical methods. For this purpose, Western blot studies on DHBV proteins were carried out.

Likewise, blocking of de novo infection of primary hepatocytes with virus which has been isolated from cells treated with proteasome inhibitors is determined by means of immunofluorescence measurement within the framework of the description of the invention. The information obtained in the process makes it possible to illustrate the inhibitory effect of proteasome inhibitors on the infection of cultured primary hepatocytes. It is demonstrated with the aid of these methods that, after treatment of infected cells with proteasome inhibitors, said cell can actually no longer release infectious virus particles. These in vitro infection studies show that all of the various substance classes of proteasome inhibitors cause the same effect: blocking of the production of infectious hepadnaviruses.

The invention further illustrates the, due to the action of the proteasome inhibitors, reduced infectivity of released immature hepatitis B virions by means of end point titration studies in primary hepatocytes. In this connection, it is demonstrated that only an incubation with proteasome inhibitors for 48 hours (approximately the time of an HBV replication cycle) results in a total blocking of the infectivity, since it was actually impossible by the methods used to observe any virus titer in the cell culture supernatants of cells which had been treated with proteasome inhibitors.

The invention demonstrates, with the example of DHBV, that the biochemical modification of the nucleocapsid protein is altered with the treatment with proteasome inhibitors. Surprisingly, it is found that core proteins which are expressed with proteasome-inhibitor treatment show a change in the molecular weight. This novel observation allows the conclusion that phosphorylation of the nucleocapsid protein has changed. From the result of this observation it is therefore concluded that the change in the modification of the nucleocapsid protein is the basic mechanism of the reduced infectivity of the DHBV secreted from the proteasome-inhibitor-treated cells.

Within the scope of the invention, it is demonstrated for the first time that, after inactivation of the UPS by treatment of chronically DHBV-infected primary hepatocytes, the release of infectious DHBV particles is surprisingly blocked. The specific infectivity of the released virions is dramatically reduced. Commonly used methods of determining infectivity cannot detect any specific titer of newly produced virus particles in the cell culture supernatants and, for this reason, a single infection assay was used (for this, see exemplary embodiment 7). To this end, according to the invention, primary duck hepatocytes which have been obtained from livers of duck embryos and cultured in vitro are infected with DHBV-containing cell culture supernatants. These DHBV-containing cell culture supernatants were obtained from chronically DHBV-infected primary duck hepatocytes which were treated with 10 microM proteasome inhibitors for two days. To a parallel culture, no inhibitors were added. At the time of maximum virus production (approx. 90% of hepatocytes of a primary liver cell culture are in the acute phase of infection), treatment of the virus-producing cultures commenced. The amount of released DHB virions is studied by means of DNA dot-blot analysis of the DNA extracted from virus particles and by means of Western blot analysis of the viral core protein. A 5- to 10-fold reduction in DHB virions released was observed.

The specific infectivity of the newly produced DHB virions is determined by means of titration on primary duck hepatocytes in permissive cultures. The amount of infected cells is determined by means of core and preS immunostaining. When using various dilutions of the cell culture supernatants for inoculation, it is found that not a single infection event is detectable in cells which have been incubated with cell culture supernatants of the cultures treated with proteasome inhibitors. A newly infected cell could not be detected in any of the cell cultures used for titration by means of immunofluorescence for DHBV core or preS proteins. According to the invention, a novel activity of proteasome inhibitors is claimed using this result (for this, see exemplary embodiment 8).

This novel activity of the proteasome inhibitors cannot be attributed to purely unspecific impairments of cell metabolism due to switching off the UPS, namely for the following reasons: this complete absence of infectious DHB virions cannot be attributed to a general inhibition of protein synthesis and DHBV expression in the producer cell, since firstly no toxic effects were observed in the primary duck hepatocytes during the 2-day treatment with proteasome inhibitors, and secondly expression analysis of intracellular DHBV proteins by means of the Western blot technique showed no significant effect of proteasome inhibition on the expression of the DHBV core protein (see exemplary embodiment 8).

The efficiency of the proteasome inhibitors with respect to blocking the proteasome pathway in the treated hepatocytes was detected by means of Western blot analysis using antibodies which recognize polyubiquitinated proteins. It is clear that in all cases of proteasome inhibitors tested there is a distinct accumulation of polyubiquitinated proteins. Thus it can be concluded that said inhibitors are fully active in primary hepatocytes.

In another embodiment of the invention it is demonstrated that treatment of chronically HBV-infected human hepatoma cell lines with proteasome inhibitors causes the release of HBV-specific proteins, in particular of HBs and HBe antigen (for this, see exemplary embodiment 10). Similarly to the action of proteasome inhibitors on DHBV, a distinct inhibition of the secretion of HBV proteins was observed.

In another form of the invention it is demonstrated that, owing to the novel action on the release and infectivity of hepatitis viruses, proteasome inhibitors prevent the spread of an HBV infection in cultured hepatocytes. According to the invention, it is detected that it is completely prevented by the secondary infection, i.e. the transfer of an already established infection to neighboring cells. This inhibitory effect on the secondary infection was detected in primary duck hepatocytes which, after primary infection, were treated with proteasome inhibitors for several days. According to the novel effects of proteasome inhibitors, both a lower expression of the viral antigens and, due to blockage of the secondary infection, a lower number of de novo infected cells were found in the primarily infected cells treated (for this, see exemplary embodiment 8).

According to the invention, this inhibitory effect of the proteasome inhibitors on DHBV secondary infection is comparable to the pharmacological action of the drug suramin which is known to block selectively an HBV secondary infection, without interfering with the already established primary infection. In contrast to the proteasome inhibitors used, however, suramin is a very toxic substance which cannot be used in vivo for the treatment of hepatitis infections. In addition, and in contrast to suramin, inhibition of the proteasome activity results additively in a reduction of HBV gene expression and causes, according to the invention, the overlapping of two antiviral effects of proteasome inhibitors, the inhibition of both the primary and the secondary infection.

Proteasome Inhibitors Induce Cell Death of Liver Carcinoma Cells, while Primary Hepatocytes are Relatively Resistant to Proteasome Inhibitors

Another part of the invention demonstrates that proteasome inhibitors induce the cell death of liver carcinoma cells. According to the invention, it is demonstrated by way of the example of cultured duck hepatocytes that primary liver cells are relatively resistant to proteasome inhibitors up to a concentration of approx. 10 microM, while liver carcinoma cells are already destroyed at 100-fold lower concentrations of proteasome inhibitors.

The induction of cell death (caused by apoptotic, necrotic or other processes) in HBV-producing liver carcinoma cells after treatment with proteasome inhibitors is illustrated according to the invention in the example of human liver carcinoma cell lines (for example HepG.2.2.15). This novel mechanism is detected by means of Trypan Blue exclusion staining (living cells are not stained, dead cells absorb the dye), by detecting annexin-V surface exposition by immuno-fluorescence, by detecting DNA fragmentation, by immunoblot detection and flubresence staining detection of processed and activated caspases and enzymic detection of caspase activity. In another embodiment of the invention, a chicken hepatoma cell line (LMH) and primary hepatocytes from ducks (DHBV-infected and noninfected) and also chickens (not infected with DHBV) are tested for selective toxicity with respect to proteasome inhibitors for hepatoma cells in cooperation with nontransformed hepatocytes.

According to the invention, the preferred death of liver carcinoma cells is caused by the antineoplastic action of proteasome inhibitors. For example, the known proteasome inhibitor PS-341 has a selective cytotoxic activity for a broad spectrum of human tumor cells, an activity which is associated with the accumulation of p21 and cell cycle arrest in the G2-M phase with subsequent apoptosis. Expression, modification and activity of the tumor suppressor protein p53 is likewise impaired by the action of proteasome inhibitors. According to the invention, it is possible for these and other mechanisms to cause the specific elimination of liver carcinoma cells which, due to an HBV infection and HCV infection or appropriate coinfection with both viruses or with HDV/HBV coinfection, are generated more than 100 times more frequently than for noninfected cells.

In another embodiment of the invention, it is found for the first time that proteasome inhibitors cause relatively low toxic effects in primary hepatocytes that lead to a preferred death of liver carcinoma cells. To this end, the invention demonstrates that primary hepatocytes tolerate days of treatment with proteasome inhibitors up to concentrations of 10 microM. In contrast, human liver carcinoma cells die already at concentrations of proteasome inhibitors 1000 times lower (for this, see exemplary embodiment 9). The different toxicity of proteasome inhibitors for primary hepatocytes compared to human hepatoblastoma cells was investigated by means of dose limitation studies with proteasome inhibitors. The vitality of the cells was checked using a light microscope. Parallel cultures were treated with increasing concentrations of proteasome inhibitors (10. microM, 3 microM, 1 microM, 10 nanoM and 1 nanoM) for several days. In addition, the functionality of the hepatocytes was determined by means of fluorescence vitality staining. According to the invention, it was found that primary duck hepatocytes can tolerate relatively high concentrations of proteasome inhibitors of up to approx. 10 microM, while proliferating liver carcinoma cells are much more sensitive to the toxic action of proteasome inhibitors.

Use of Proteasome Inhibitors in the Treatment of infections with Hepatitis Viruses

The principle of using proteasome inhibitors for blocking an infection with hepadnaviruses, illustrated in the description of the invention, is novel with regard to the use of an already known substance class (the proteasome inhibitors) for a new activity which can be summarized in the following therapeutic concepts:

blocking of the production of infectious hepadnaviruses and thus preventing the spread of the infection in vivo, in the liver tissue of an infected individual;

inducing the death of liver carcinoma cells which have been generated as a direct or indirect result of an infection with hepadnaviruses.

At the same time, the use of proteasome inhibitors is also novel with regard to the administration principle. Up until now, no substances/principles/methods are known which influence late processes of hepadnavirus replication, especially the release of infectious virions. Another novelty is the fact that the use of proteasome inhibitors results in the blocking of hepatitis virus replication. This mechanism of inhibition is conserved in all virus host cells, the liver hepatocytes. In comparison with previous antiviral methods of treating hepatitis infections, which affect essential viral components directly, the probability of the development of resistance mechanisms in the case of administration of proteasome inhibitors in the treatment of hepadnavirus infections is lower by order of magnitudes. The novelty of this principle of action of proteasome inhibitors is also obvious in the fact that proteasome inhibitors have a broad spectrum of action on different hepatitis viruses (HAV, HBV, HCV, HDV, HEV, HGV). The same intensity of the inhibitory effect was observed within the framework of the invention in various primary as well as cloned hepatitis viruses.

Another novelty is the principle of action of proteasome inhibitors which prevent the production of infectious virus particles from cells already infected with hepadnaviruses. This substantially reduces the amount of infectious virions (viral load) and thus the spread of the infection in vivo.

In summary of these novel mechanisms, it can be concluded that, in the case of an in vitro administration of proteasome inhibitors, the net effect of the reduced release of, moreover, a few virus particles or virus particles which are not infectious at all with the simultaneous cell death of virus-producing carcinoma cells is a reduction in the amount of infectious virions in an organism infected with hepadnaviruses. Thus, the total number of infected producer cells in the liver cell tissue is reduced. This renders effective the administration of proteasome inhibitors alone or in combination with therapeutics already used in the antiviral therapy of hepadnaviruses.

Within the scope of the invention, it is found for the first time that proteasome inhibitors both inhibit maintenance and persistence of an already established infection and completely block in hepatocytes the secondary infection and thus the spread of an infection with hepatitis viruses in vivo. According to the present invention, proteasome inhibitors are substances suitable for blocking the spread of an HBV infection in vivo.

The invention has the substantial advantage that, during treatment, proteasome inhibitors can produce two effects important for controlling viral hepatitis infections: firstly, production of infectious virus particles and thus the spread of the infection in the organism are inhibited. Secondly, the development, growth and metastasizing of liver cell tumors, the latter of which occurs, after a latency phase, very frequently following a hepatitis virus infection, are prevented. In addition, the proteasome inhibitors destroy liver carcinomas already present but not the low-proliferating or nonproliferating normal cells of the liver.

Owing to this novel method of treatment, it is therefore possible to produce a multiplicity of therapeutic effects by using proteasome inhibitors in infections with hepadnaviruses. In addition to blocking the infectivity of the released viruses and preventing liver cell carcinomas, said method of treatment has another advantage in that this strategy affects cellular factors which are essential for the replication of hepadnaviruses but which comprise a very much higher genetic stability compared to viral factors. Owing to said genetic stability of the target structure of this novel antiviral strategy, the appearance of resistance symptoms, as known for many of the previously known inhibitors of an HBV infection, is not a factor. This applies, in particular, to the polymerase mutants of hepatitis B and C viruses, which, in the case of nucleoside analog treatment, practically always appear after a relatively short time. This applies also to immunoescape variants as appearing in passively and actively vaccinated patients as well as naturally. The same applies to interferon-resistant strains of HBV and HCV. This novel effect of proteasome inhibitors is the basis of the inventive claim that treatment with proteasome inhibitors makes it possible to not only prevent the spread of an HBV infection but also to treat liver cell carcinomas caused by HBV.

The invention is intended to be illustrated in more detail on the basis of exemplary embodiments, without being limited to these examples.

### EXEMPLARY EMBODIMENTS

### EXAMPLE 1

Treatment of HIV-1-Infected Cells with Proteasome Inhibitors Reduces the Infectivity of Released Virus Particles.

Cultures of human CD4⁺ T cells, A3.01 were infected with HIV-1_{NL4-3} and cultured in RPMI for approx. 7 days. Approx. 80% of the cell culture medium was replaced every other day. The amount of released virus particles was determined by testing samples of the cell culture supernatants for activity of virus-associated RT. Furthermore, the spread of the infection was monitored by indirect immunofluorescence with anti-CA antibodies. The time of maximum spread of the infection in the culture was determined on the basis of RT accumulation and of syncytia formation. At the time of maximum virus production (approx. 7 days after infection), fresh noninfected A3.01 cells were mixed with the infected culture in a 1:1 ratio and incubated for another 24 h. This makes it possible to quasi-synchronize the infection and to maximize the number of infected cells with maximum HIV expression at a given time. The cells were divided into parallel cultures, washed with PBS and treated with 40 microM zLLL in medium for 4.5 hours. Washing of the cells and culturing in fresh medium was necessary in order to monitor the newly produced viruses during the harvest period of 4.5 hours (+zLLL “0 hr”). It was assumed that there is a certain lag phase of approx. 1 hour, corresponding to HIV-1 assembly, between the start of the treatment with proteasome inhibitors and the release of defective virions. In order to monitor the virus production of cells with inactivated proteasomes at the start of the harvest period, parallel cultures were pretreated with 40 microM zLLL prior to the washing step for one (+zLLL “−1 hr”) or 6 hours (+zLLL “−6 hr”). A parallel culture was incubated without inhibitor (no zLLL). The virus-containing, cell culture supernatants were collected, filtered, and the amount of Gag proteins was quantified by means of a standardized capture ELISA using anti-CA antibodies with different epitopes. The virus release calculated therefrom is displayed as nanog of p24^{CA}/ml of cell culture medium in Table 1. The infectivity of the culture supernatants was determined by means of end point titration (detailed in example 6d) and is indicated as TC_{ID50} in Table 1. The specific infectivity was determined by dividing TC_{ID50} by the total amount of Gag and is listed in Table 1 as percentage infectivity of the control culture (no zLLL=100%). Both the release of Gag proteins and the infectivity of the released virions are shown to be inhibited after zLLL treatment. This effect increases with time, and infectious virions in the cell culture medium are reduced by 84% after 4.5 hours and by 98% after another 6 hours of pretreatment (Table 1).

CD4⁺ T cells (A3.01) were infected with HIV-1_{NL4-3}, at the time of maximum virus production (approx. 7 days post infection), parallel cultures were treated either without or with 40 μM zLLL for 1 (+zLLL “−1 hr”) or 6 hours (+zLLL “−6 hr”). This was followed by washing the cells and another 4.5 hours of incubating with or without 40 μM zLLL. In a parallel culture, cells were treated with 40 μM zLLL immediately after washing (+zLLL “0 hr”). The virus-containing supernatants were collected, and the amount of CA antigen was quantified by means of ELISA. The specific infectivity was determined as the infectious virus titer per nanog of CA and depicted relative to the untreated control culture (100%).

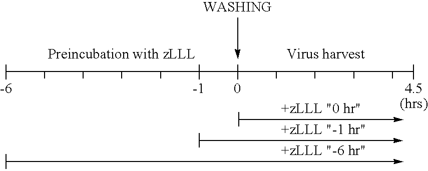

| | Virus | | | |
|---|---|---|---|---|
| | release | Virus titer/ | Infectivity | Infectivity |
| | ng p24^{CA}/ | ml | Titer/ | % of |
| Treatment | ml | TC_{ID50} | ng p24^{CA} | “no zLLL” |
| no zLLL | 263 | 1.9 × 10⁷ | 7.2 × 10⁴ | 100 |
| +zLLL, “0 hr” | 122 | 1.2 × 10⁶ | 1.0 × 10⁴ | 14 |
| +zLLL, “−1 hr” | 88 | 4.4 × 10⁵ | 5.0 × 10³ | 7 |
| +zLLL, “−6 hr” | 26 | 3.8 × 10⁴ | 1.5 × 10³ | 2 |

### EXAMPLE 2

Electron-Microscopic Analysis of HIV-1-Infected MT-4 Cells After Treatment with Proteasome Inhibitors

CD4⁺ T cells, MT-4, were infected with HIV-1_{NL4-3} and cultured in RPMI for approx. 4 days. At the time of maximum virus production, the cells were washed, sucked into cellulose capillaries and treated with 50 μM (micromol) zLLL. The experimental details of fixing, preparation of thin sections and transmission electron microscopy are described in Example 6e. FIG. 1 depicts representative sections of electron-microscopic images.

### EXAMPLE 3

Proteasome Inhibitors Inhibit Gag Processing and Virus Release from Infected T-Cell Cultures and Transfected HeLa Cells.

The inhibitory action of proteasome inhibitors on the kinetics of Gag processing and virus release was biochemically analyzed by carrying out pulse/chase analyses. The experimental details of infection, culturing, DNA transfection and pulse/chase experiments are described in Example 6g. To this end, either HIV-1-infected CD4⁺ T-cell cultures or cultures of HeLa cells were used, which were transfected with proviral DNA of HIV-1_{NL4-3} or HIV-2_{ROD10}. Usually, parallel cultures were subjected to methionine depletion for 30 min at the time of maximum expression of HIV proteins, then metabolically pulse-labeled with [³⁵S]-labeled methionine for 30 min and subsequently incubated in a chase medium with an excess of nonradiolabeled methionine for a period of 8 hours. The treatment with proteasome inhibitors usually began at the time of methionine depletion and was maintained over the entire period of the experiment (depletion, pulse and chase phases). Proteasome inhibitors were used either selectively (10 microM zLLL, FIG. 2, panel “HIV-1 in HeLa”) or in combination (10 microM zLLL and 10 microM LC, FIG. 2, panels “HIV-1 in HeLa” and “HIV-2 in HeLa”). Cell culture aliquots were obtained at each point in time of the chase and fractionated by centrifugation into cellular, viral and cell culture-supernatant fractions. The radiolabeled HIV proteins were isolated by means of standard immunoprecipitation using sera of AIDS patients and Gag-specific antibodies and using methods already described (Schubert et al., 1998), fractionated in an SDS-PAGE, and then made visible by means of fluorography. Quantification is carried out by means of image analysis. Usually, the relative amounts of Gag polyprotein and of the main processing product CA (capsid) were determined for each point in time of the chase in each case in the cellular, viral and the cell culture fractions. The kinetics of virus release were depicted as the percentage of Gag proteins in the viral fraction relative to the total amount of Gag (determined in the cellular, viral and cell culture fractions) per point in time of the chase. The kinetics of intracellular Gag processing were calculated by dividing the amount of CA by the amount of Pr55 over the entire chase period (FIG. 2, panel “HIV-1 in HeLa”).

### EXAMPLE 4

In Vitro Gag Processing of Pr55

Recombinant HIV-1 Gag polyprotein Pr55 was prepared in insect cells and recombinant HIV-1 protease was prepared in E. coli. The experimental details of expression, purification and determination of the enzymic activity as well as carrying out the in vitro cleavage reactions and Western blots are illustrated in more detail in Example 6f. The enzyme-to-substrate ratios (protease-Pr55 ratio) were chosen in such a way that substrate conversion was relatively slow. After 30 min of reaction, approx. 50% of Pr55 had been cleaved. Under these conditions it is possible to determine even weakly inhibitory effects on the enzymic activity of the protease. Under these sensitive conditions, it was not possible to detect any inhibition of protease activity, not even under conditions under which both inhibitors, zLLL and LC, were tested at a 10-fold higher concentration than in the cell culture (FIG. 3, reactions 4-9). As control: the HIV-1-specific proteasome inhibitor saquinavir causes a complete blockage of in vitro processing (FIG. 3 reaction 10).

### EXAMPLE 5

Proteasome Inhibitors Inhibit HIV-1 Replication in Cell Culture.

Parallel cultures of CD4⁺ T cells were incubated in fresh RPMI medium and infected with a defined virus stock of purified HIV-1_{NL4-3}. Usually, 1 RT unit per cell was used for infection. One day after infection, the cells were washed in PBS, admixed with fresh medium which [lacuna] various concentrations of the proteasome inhibitors zLLL (FIG. 4, panels “1st to 3rd experiment in A3.01”) or epoxomicin (FIG. 4, panel “4th experiment in A3.01”).

Every other day, samples were obtained from cell culture supernatants, frozen and used later for determining RT activity. At the same time, 80% of cell culture medium was replaced and admixed with fresh proteasome inhibitors. Said proteasome inhibitors were employed as 10 mM (millimol) stock solutions in 75% ethanol. As a negative control, a parallel culture was admixed with 20 microM of ethanol. Reverse-transcriptase activity was determined in the cell-free cell culture supernatants and plotted as a function of cell culture time (FIG. 4).

### EXAMPLE 6

Materials and Methods

### EXAMPLE 6a

Molecular HIV-1 Clones

The previously published plasmid pNL4-3 (Adachi et al., 1986) was used for preparing T cell-tropic viruses of the molecular clone HIV-1_{NL4-3}, and the previously published plasmid pNL4-3(AD8) (Schubert et al., 1995) and pAD8 (Theodore et al., 1995) were used for preparing macrophage-tropic viruses. The previously published plasmid pROD10 (Bour et al., 1996) was used for expressing HIV-2_{ROD10} in HeLa cells.

### EXAMPLE 6b

Cell Culture

CD4⁺ human T-cell lymphoma cell lines, H9, A3.01, C8166, and MT4, were cultured in RPMI 1640 containing 10% (v/v) fetal calf serum, 2 milliM L-glutamine, 100 U ml⁻¹ penicillin and 100 mg (millig) ml⁻¹ streptomycin. HeLa cells (ATCC CCL2) were cultured in Dulbecco's modified Eagle's medium (DMEM) containing 10% (v/v) fetal calf serum, 2 milliM L-glutamine, 100 U ml⁻¹ penicillin and 100 mg ml⁻¹ streptomycin.

### EXAMPLE 6c

Transfection and Obtaining Virus Stock

Viral preparations were prepared by transfecting plasmid DNA of molecular HIV-1 or HIV-2 DNA into HeLa cells by applying the calcium phosphate precipitation method. For this purpose, confluent HeLa cell cultures (5×10⁶ cells) were incubated with 25 μg (microg) of plasmid DNA, prepared in calcium phosphate crystals, and then subjected to a glycerol shock. Concentrated viral preparations were obtained by harvesting the cell culture supernatants two days after transfection. The cells and components thereof were then removed by centrifugation (1000×g, 5 min, 4° C.) and filtration (pore size 0.45 μm—microm). Virus particles were pelleted by means of ultracentrifugation (Beckman SW 55 rotor, 1.5 hr, 35 000 rpm, 10° C.) and subsequently resuspended in 1 ml of DMEM medium. The viral preparations were sterilized by filtration (pore size 0.45 microm) and frozen in aliquots (−80° C.). Individual viral preparations were standardized by determining the reverse-transcriptase activity using [³²P]-TTP incorporation into an oligo(dT)-poly(A) template.

### EXAMPLE 6d

Infectivity Assay

Acute HIV-1_{NL4-3}-infected A3.01 cells were treated with 40 microM zLLL for various times, as indicated in exemplary embodiment 1. Cell-free cell culture supernatant was harvested and filtered (pore size=0.45 microM). The relative quantity of released virions was quantified by means of a p24^{CA}-antigen capture ELISA. For the purpose of virus titration, in each case 8 parallel cultures of C8166 cells were infected with the particular viral preparations, in a serial 10-fold dilution. The infectivity titer was determined as 50% cell culture infectious dose (TCID₅₀), namely by determining syncytia formation for each culture and dilution step on day 10 post infection. The specific infectivity was standardized for each sample to the quantity of 24^{CA} antigens determined in each viral inoculum.

### EXAMPLE 6e

Electron Microscopy

MT-4 cells acutely infected with HIV-1_{NL4-3} were cultured in fresh medium with proteasome inhibitor (50 microM zLLL) or in a parallel culture without proteasome inhibitor for 2.5 hours. The cells were then sucked into cellulose capillaries, the capillaries were sealed and cultured further in fresh medium with or without 50 microM zLLL for another 2.5 hours. The capillaries were washed, and the cells were incubated in 2.5% glutaraldehyde in phosphate-buffered saline (PBS) for one hour. The capillaries were then washed in PBS and subsequently fixed in 1% OsO₄ in PBS, washed again in water, stained in 1% uranyl acetate in water, and finally dehydratized in an increasing gradient of serial dilutions of ethanol. All capillaries were embedded in ERL resin for subsequent ultra thin sections. Said ultra thin sections were counterstained with 2% uranyl acetate and lead citrate. Microimages were recorded in a Philips CM 120 transmission electron microscope at 80 kV.

### EXAMPLE 6f

In Vitro Processing of Gag Proteins

Myristylated Pr55 of HIV-1 was produced from virus-like particles which were released from insect cells infected with recombinant baculovirus Gag12myr. The virus-like particles were purified by centrifugation through a 20% sucrose layer. Recombinant HIV-1 protease expressed in E. coli was purified by means of gel filtration chromatography on Superose 12. The enzyme concentration was determined by means of active-site titration. For cleavage reactions, usually 1 μM Pr55 was incubated with 40 nM (nanomol) PR in reaction buffer (50 mM MES pH 6.5, 300 mM NaCl, 2 mM DTT, 1 mM EDTA, 0.1% Triton-X 100), at 37° C. for 30 min. In order to ensure maximum saturation of PR with inhibitors, PR was preincubated with the particular inhibitor for 5 min, prior to the start of the cleavage reactions. The cleavage reactions were stopped by adding SDS sample buffer and boiling the sample for 10 min. The cleavage products were fractionated in 10% SDS-PAGE and then detected by means of Western blot. For this purpose, anti-CA-specific antibodies were used which were detected by means of anti-rabbit IgG-peroxidase conjugate and chemiluminescence reaction.

### EXAMPLE 6g

Metabolic Labeling, Pulse/Chase Analysis and Immunoprecipitation

For pulse/chase analyses, HIV-1- or HIV-2-expressing cells, either infected T cells or transfected HeLa cells, were incubated in methionine-free RPMI medium for 30 min (methionine depletion). This was followed by metabolic pulse labeling with [³⁵S]-methionine (specific activity=2 milliCi/ml) in RPMI for 30 min. The cells were washed in PBS, divided into aliquots and further incubated in parallel cultures in complete DMEM containing 10 milliM nonradioactive methionine (chase). At particular times of the chase (usually 0, 0.5, 1, 2, 4 and 8 hours after pulse labeling), the cells were harvested and, immediately thereafter, frozen on dry ice. The cell-free virions were isolated by centrifugation of the cell culture supernatants (at 4° C. and 18 000×g, for 100 min.). Extracts of cell and virus pellets were prepared by means of detergents and aliquots of the cellular, viral and cell culture supernatants, obtained after centrifugation, were treated with antibodies which had previously been coupled to protein-G Sepharose. Normally, serum of HIV-1- or HIV-2-seropositive individuals as well as anti-CA antibodies prepared in rabbits were used for immuno-precipitation. The immunoprecipitates were washed with detergent-containing buffers, denatured in SDS sample buffer and then fractionated by means of gel electrophoresis in 10% SDS-PAGE. The gels were fixed in solutions of 50% methanol and 10% acetic acid and then treated in 1M salicylic acid for 1 hour. The radiolabeled proteins were made visible by fluorography at −80° C. The relative amount of the labeled proteins, especially of Gag polyproteins and CA-processing products, was determined by means of a phosphor image analyzer.

### EXAMPLE 7

Inhibition of Proteasome Activity has no Substantial Influence on Intracellular Viral Gene Expression, but Blocks Drastically the Release of Virus Particles from Chronically DHBV-Infected Hepatocytes.

Primary duck hepatocytes and other nonparenchymal cells of the liver were obtained from duck embryos by means of collagenase treatment essentially according to a previously published method (Köck et al., 1993). Fertilized Peking duck eggs were purchased from a commercial breeder (Wichmann, Germany). The eggs were hatched in an automated incubator at 37° C. and 50% humidity for at least 21 days. After opening the egg and removing the embryo, said embryo was sacrificed and blood was removed. Subsequently, the liver was removed, excluding the gall bladder, chopped up mechanically and taken up in 3 ml of Williams E medium (GIBCOBRL, Paisley, Scotland) containing 0.5% collagenase (Sigma, Deisenhofen, Germany) and digested at 37° C. for 1 h. The cells were-washed twice with native Williams E medium, the vital and dissociated cells were separated from larger cell aggregates and cell debris by 10% strength Percoll density centrifugation (Sigma, Deisenhofen, Germany). In parallel, the blood sera removed were assayed for the presence of a natural congenital DHBV infection by means of protein dot-blot analysis using preS antiserum (Sunjach et al., 1999). Hepatocytes from DHBV-positive animals were pooled and used in this experiment. The cells were resuspended in Williams E medium (GIBCOBRL, Paisley, Scotland), supplemented with 2 milliM L-glutamine (GIBCOBRL, Paisley, Scotland), 100 units ml⁻¹ penicillin, and 100 microg ml⁻¹ streptomycin (Biochrom, Berlin, Germany), 15 mM HEPES (N-[2-hydroxyethyl]piperazine-N′-[2-ethanesulfonic acid]) (pH 7.2) (all from GIBCOBRL, Paisley, Scotland), 10⁻⁵ hydrocortisone, 10⁻⁹ M insulin and 1.5% DMSO (all from Sigma, Deisenhofen, Germany) and seeded into 12-well microtiter plates (Greiner, Solingen, Germany) with a density of approx. 8×10⁵ per well and cultured at 37° C. The medium was replaced approx. 4 h after plating out, and again 24 h later. Unless stated otherwise, the medium was then replaced every 2 to 3 days. One week after seeding the mixture of primary hepatocytes and other liver cells, the culture medium was again replaced, and the cells were incubated with new medium which now contained different concentrations of PI (10 microM, 3 microM, 1 microM, 10 nanoM, and 1 nanoM) or no inhibitor. PI was dissolved in PBS as described (Adams and Stein, 1996, Adams et al., 1996), and an aliquot of the 1000 times-concentrated stock solution was added to the medium. The morphology of the cells during the treatment phase was checked under a light microscope every 12 h (hours). The first morphological changes indicating toxicity were observed only at a concentration of 10 microM PI. Said changes appeared after approximately 48 h of treatment and comprised fine-granule vacuolization, loss of macrovacuolar lipid droplets and flattening of the hepatocytes, while in nonparenchymal cells, rounding, shrinking and detachment from the base of the cell culture dish dominated the picture. For concentrations less than 10 microM PI, no significant morphological changes which would indicate PI-induced changes in cell metabolism were observed in hepatocytes even after 48 h of treatment.

In contrast, continuous treatment with 1 microM PI for 72 h was not compatible with the vitality of the nonparenchymal cells. The phase contrast image (FIG. 5) shows a relatively large region with a former cluster of nonparenchymal cells, in which, after treatment with 1 microM PI, hardly any adherent cells are present any more. In contrast, the hepatocytes have maintained their normal morphology (FIG. 5, block arrow). The Hoechst staining (FIG. 5) proves that the remaining nonparenchymal cells are rounded and pyknotic (arrows).

After treating the chronically DHBV-infected hepatocytes with different doses of PI (10 microM, 3 microM, 1 microM, 10 nanoM, and 1 nanoM) or no inhibitor for 48 h, cell culture supernatants were harvested and centrifuged (4000 rpm for 5 min). The amount of the viral particles released was determined by means of a preS antigen-specific dot blot (FIG. 6) and an immunoblot (FIG. 7) (Bruns et al., 1998). For dot-blot analysis, aliquots of 300 microl were dotted onto a nitrocellulose membrane (Schleicher & Schuell, Dassel, Germany) by means of an apparatus (Schleicher & Schuell, Dassel, Germany), dried in air and then washed with PBS (phosphate-buffered saline). For Western blot analysis, 5 μl of supernatant were denatured by boiling for 5 min, after adding SDS-PAGE sample buffer. Subsequently, the samples were fractionated in 12.5% SDS-PAGE and electrotransferred to nitrocellulose by means of the semi-dry transfer technique (Biorad, Munich, Germany).

Blocking with 5% skimmed milk was followed by incubating the membranes with preS-specific antiserum (rabbit anti-preS Kpn; Fernholz et al., 1993). The labeled proteins were made visible by means of the enhanced chemiluminescence (ECL) method (Pierce, Rockford, USA). FIGS. 6 and 7 indicate that proteasome-inhibitor treatment of DHBV-infected hepatocyte cultures drastically reduces the amount of preS protein in the medium in a dose-dependent manner. Even at a relatively low concentration of 0.01 microM PI, an almost 10-fold inhibition of the release of the subviral particles was observed (FIG. 7). Since preS protein is a structural component of all viral particles (empty subviral particles and DNA-containing infectious virions), the amount of preS protein in the dot and Western blots directly reflect the amount of enveloped virus particles which were secreted into the medium during the treatment period.

The effect of proteasome inhibitors on the amount of the genome-containing viral particles released into the medium was analyzed by means of DNA dot blot (Sunjach et al., 1999). After treating the chronically DHBV-infected hepatocytes with different doses of PI (10 microM, 3 microM, 1 microM, and 10 nanoM) or with no inhibitor for 48 h, the cell culture supernatants were harvested and centrifuged (4000 rpm for 5 min) After dotting samples (in each case 200 microl) onto a nitrocellulose membrane, said membrane was denatured in an alkaline buffer (0.5 M NaOH and 1.5 M NaCl) on 3 MM Whatman paper (Schleicher & Schuell, Dassel, Germany) twice for 1.5 min each time and then renatured using a neutral buffer (0.5 M Tris.HCl, pH 7.4 and 3M NaCl) on 3 MM Whatman 4 times for 1 min each time. After drying in air and crosslinking of the membrane, the latter was then hybridized with ³²P-labeled cloned DHBV genome as probe. The signals were made visible by means of autoradiography according to a standard protocol (Sunjach et al., 1999). FIG. 8 shows that PI reduces the amount of the released, complete virus particles by at least a factor of approx. 4. The DNA determination result thus correlates with the amount of secreted preS antigen reduced in a similar manner (FIGS. 6 and 7).

In order to assay the influence of the treatment with proteasome inhibitors on intercellular DHBV expression, the parallel treated and untreated cells were lysed with 200 microl of buffer (50 mM Tris.HCl pH 7.5, 150 mM NaCl, 1% Triton X-100, 1% sodium deoxycholate) complemented with a protease-inhibitor cocktail (Complete, Roche, Mannheim). The lysates were separated from the insoluble cell fraction by centrifugation (14 000 rpm, 10 min, 4° C.) and in each case 20 microl of the clarified total lysates were denatured, after adding Laemmli buffer, by boiling for 5 min. The samples were then fractionated in 12.5% SDS-PAGE and transferred to nitrocellulose by means of the semi-dry transfer technique (Biorad, Munich, Germany). The membranes were blocked with 5% skimmed milk (Biorad, Munich, Germany) and, after washing with TBS (Tris-buffered saline), incubated with antibodies against preS protein (Fernholz et al., 1993) or against core protein (Breiner et al., 2001) (FIGS. 9 and 10). The labeled viral proteins were then made visible by means of enhanced chemiluminescence (Pierce, Rockford, USA).

FIGS. 9 and 10 indicate that treatment with proteasome inhibitors does not lead to any substantial change in the steady-state amounts of intracellular preS protein (FIG. 9) and core protein (FIG. 10). Only at the highest concentration of 10 microM PI, a slight reduction in viral proteins is observed. It is remarkable that, for example, 10 nanoM PI has no effect whatsoever on the intracellular viral protein expression but still exerts a distinct effect on the release.

In summary of these results, it is concluded that the treatment of chronically infected primary duck hepatocytes with PI up to a concentration of 10 microM over a treatment period of 48 h has only a slight, if any, influence on the intracellular expression of core antigen and preS antigen, whereas the release of viral particles, however, is drastically reduced. In addition, it can be concluded that vitality and protein synthesis of said primary duck hepatocytes are not substantially impaired.

In order to demonstrate the efficacy of the treatment of primary duck hepatocytes with proteasome inhibitors with regard to blockage of the UPS, in each case 20 microl of the cell lysates were taken up in SDS-PAGE sample buffer, boiled for 5 min and fractionated in 6% SDS-PAGE. The proteins were transferred to nitrocellulose and incubated with polyclonal rabbit anti-ubiquitin antibodies (Sigma, Deisenhofen, Germany) and made visible by means of a subsequent ECL reaction.

FIG. 11. indicates, from a concentration of 1 microM PI, a distinct accumulation of polyubiquitinated proteins in relation to the lower molecular weight bands. It is known that long-term treatment with proteasome inhibitors leads to a change in both protein synthesis and protein pattern. This is clearly visible on the basis of the altered profile of the lower molecular weight proteins in the PI-treated cells and is therefore further proof of the efficacy of PI for efficient blocking of the proteasome activity in primary hepatocytes.

### EXAMPLE 8

Proteasome Inhibitors Block the Production-/Release of Infectious Viruses from Chronically DHBV-Infected Primary Hepatocytes.

In the preceding experiments (Example 7), it was found that inhibition of the cellular proteasome activity substantially restricts the release of DHBV proteins, without, however, substantially influencing intracellular viral protein expression. It therefore remained still to be clarified whether treatment with proteasome inhibitors not only reduces the secretion of virus particles by a factor of approx. 4 but, in addition, also still have an adverse effect on the degree of infectivity of progeny viruses. We therefore investigated whether and how many infectious virions are contained in the supernatants of chronically DHBV-infected primary duck hepatocytes treated with proteasome inhibitors, compared to untreated cells. To this end, approx. 7-day-old, chronically DHBV-infected primary duck hepatocytes whose preparation has already been described under Example 7 were treated with 10 microM PI for 48 h. As already described under Example 7, the cell culture supernatants were then harvested and utilized for de novo infection of primary liver cells. DHBV-negative, primary duck hepatocytes were prepared as described in Example 7 and seeded into 12-well microtiter plates with a density of approx. 8×10⁵/well. A week after plating out, the cells were infected by adding 1 ml of cell-free cell culture supernatants of PI-treated or untreated DHBV-positive cell cultures. A parallel culture was infected with 20 microl of DHBV-positive serum/well as a control (corresponds to a multiplicity of infection (MOI) of 20). After 16 h of incubation at 37° C., the virus inoculum was removed, the cells were washed with PBS and then cultured further in new medium. Establishment of a productive infection was determined by means of indirect immunofluorescence and SDS-PAGE for the viral proteins Core and PreS after 2.5 days. For immunofluorescence staining, the cells were washed with PBS and then fixed with 1 ml of an ice-cold 1:1 mixture of methanol and acetone at room temperature for 10 min. The fixed cells were incubated with the first antibody, rabbit anti-PreS (Breiner et al., 2001) at a dilution of 1:800 in PBS at room temperature for 1 h. Washing with PBS was followed by 30 minutes of incubation with the Alexa 488-coupled secondary antibody (goat anti-rabbit, Molecular Probes, Leiden, The Netherlands). The nuclei were stained with Hoechst (4 microg/ml) (Sigma, Deisenhofen, Germany). The fluorescence signals were analyzed using an inverse epifluorescence microscope (Axiovert S100, Carl Zeiss, Göttingen, Germany) and processed by means of the image processing system Openlab (Improvision, Coventry, UK). The immuno-fluorescence images show that no preS-expressing cell was detected in cell cultures which had been infected with DHBV from cell culture supernatants of PI-treated cells (FIG. 12, preS), while approx. 1-5% of the hepatocytes in those cultures incubated with supernatants of untreated cells were unambiguously preS-positive and were thus infected (FIG. 13, preS). The number of cells present on the slide was detected by staining the nuclei thereof with Hoechst and overlaying this image with the fluorescence images (for treated cells, see FIG. 12 and for untreated cells, see FIG. 13, in each case Hoechst and preS merge).

For Western blot analyses, the cells from parallel cultures were lysed directly in 200 microl of Laemmli buffer and boiled for 5 min. In each case, 20 μl of the cell lysates were fractionated in 12.5% SDS-PAGE and transferred to nitrocellulose. The membranes were incubated with antibodies against preS protein (Fernholz et al., 1993) or against core protein (Breiner et al., 2001), and proteins were made visible by ECL. The blots show clearly visible signals for preS protein (FIG. 14, lane 3) and core protein (FIG. 15, lane 3) in the extracts of cells infected with DHBV from untreated cells, but practically no indication of expression of DHBV proteins in cells incubated with cell culture supernatants of PI-treated cells (FIG. 14, lane 4 and FIG. 15, lane 4, respectively).

### EXAMPLE 9

Proteasome Inhibitors Block Both the Early Phase of an Acute DHBV Infection and the Subsequent Secondary Infection and thus the Spread Thereof in Cell Culture.

In the previous exemplary embodiments, it was shown that proteasome inhibitors not only inhibit the secretion of new viral and subviral particles from chronically DHBV-infected hepatocytes but, in addition, also virtually completely block the infectivity of the few DHBV virions still released. It can therefore be assumed that proteasome inhibitors can prevent the spread of an already established infection (by progeny viruses released from already infected hepatocytes) to noninfected cells, the “secondary infection”. This effect was studied in the following experiment: primary hepatocytes from DHBV-negative animals were prepared exactly according to the methods described in Example 7. 4 days after seeding the cells into 12-well microtiter plates (approx. 8×10⁵ cells/well), the culture medium was replaced again and the cells were infected at an MOI of 20. After incubating for 16 h, the cells were washed with PBS and then further cultured in new medium for another 3 days. During this period, only the primary infection is established. The medium was then replaced again, and in each case 1 μM of the proteasome inhibitor eponemycin or epoxomicin was added to parallel cultures and said cultures were incubated for another 3 days. According to the above-described effect of proteasome inhibitors, the treated primarily infected cells should, in comparison with untreated cells, exhibit lower expression of the viral antigens and (due to blockage of the secondary infection) the number of infected cells should be markedly reduced. In order to test this, the cell cultures were fixed and the number of core-positive cells and level of expression thereof were determined at the single-cell level by indirect immunofluorescence with anti-core antibodies. The proteasome inhibitors used indeed inhibit both the maintenance of an already established productive infection (evident in FIG. 16 (core, PI, epone- and epoxomicin) which is indicated by the much lower number of core-positive cells compared to untreated cultures (FIG. 16, untreated). For comparison, phase contrast images (FIG. 16, phase contrast) and Hoechst staining (FIG. 16, Hoechst) of the same image sections are displayed.

In order to find out whether proteasome inhibitors inhibit the release of the “e-antigen” which is required for establishing a chronic infection (Chen et al., 1992), the amount of the e-antigen in the medium was determined by means of immunoblot and correlated with the likewise tested preS antigen. To this end, the proteins of one aliquot from each of the cell culture media (20 microl) were fractionated in 12.5% SDS-PAGE, transferred to nitrocellulose, and the e- and preS-antigen proteins were detected by rabbit antibodies against core proteins (crossreacting with the e-antigen, Breiner et al., 2001) and, respectively, preS by means of ECL. As FIGS. 17 and 18 show, proteasome-inhibitor treatment leads to a substantial reduction in the amount of the e- and preS antigens in the medium. Coomasie blue staining of the gels revealed that the proteasome inhibitors inhibit specifically only the secretion of the viral proteins tested but not that of particular cellular proteins.

In summary, these results demonstrate that treatment with proteasome inhibitors leads to a drastic reduction in the release of the e- and preS antigens.

In order to show that the lower number of DHBV-positive cells in the cells treated with proteasome inhibitors is indeed caused by blockage of the secondary infection, the cells were treated, three days after infection, with 100 microg/ml suramin (Sigma, Deisenhofen, Germany) for three days. Suramin, a very toxic substance in vivo, is known to block the secondary infection, without interfering with the already established primary DHBV infection (Pugh and Simmons, 1994). On the other hand, hepatocytes pretreated with suramin are resistant to DHBV infection. Accordingly, the suramin-treated cells exhibit, three days after the primary infection (FIG. 19, suramin 3 d post-infection), a core staining whose intensity is similar to that in the untreated cultures (FIG. 19, untreated), but the number of the core-expressing cells is very greatly reduced. As expected, pretreating the cells with suramin completely blocks the establishment of the primary infection (FIG. 19, suramin 2 h pre-infection). The corresponding phase contrast images (FIG. 19, phase contrast) and Hoechst stainings (FIG. 19, Hoechst) of the same image sections are likewise displayed, for comparison.

In summary, these experiments demonstrate that proteasome inhibitors, similarly to suramin, can prevent the spread of the DHBV infection by blocking the secondary infection. In addition, and in contrast to suramin, inhibition of the proteasome activity leads additively to a reduction in viral gene expression in primarily infected hepatocytes. The effects of proteasome inhibitors both on the primary infection and on the secondary infection correlate closely with the potential of the different substance classes used.

Overall, these experiments demonstrate that proteasome inhibitors inhibit both the establishment and the persistence of a primary infection. In addition, they prevent the spread of the primary DHBV infection by blocking the progeny viruses. As a result, proteasome inhibitors are substances suitable for blocking the spread of an HBV infection in vivo.

### EXAMPLE 10

Proteasome Inhibitors Induce Hypophosphorylation of the Core Protein in Chronically DHBV-Infected Primary Hepatocytes.

In the previous exemplary embodiments, it was shown that proteasome inhibitors not only inhibit the secretion of new viral and subviral particles from hepatocytes but also, in addition, block virtually completely the infectivity of the few DHB virions still released. One reason for the massively reduced infectivity of DHBV could be based on the proteasome inhibitor-mediated posttranslational modifications of the structural components of the virus. For example, a change in the degree of phosphorylation of the core protein would result in a defect in the destabilization of “incoming cores” in the early phase of the infection.

In order to assay the influence of the treatment with proteasome inhibitors on the intracellular phosphorylation pattern of the core protein, approx. 7-day-old, chronically DHBV-infected primary duck hepatocytes were treated with increasing PI concentrations of 10 μM, 3 μM, 1 μM and 10 nM. Finally, the cells were lysed (50 mM Tris.HCl pH 7.5, 150 mM NaCl, 1% Triton X-100, 1% sodium deoxycholate, complemented with a protease-inhibitor cocktail) (Complete, Roche, Mannheim)). The cell lysates were separated from the insoluble fraction by centrifugation (14 000 rpm, 10 min, 4° C.) and in each case 20 microl of the de novo fraction were denatured, after adding Laemmli buffer, by boiling for 5 min. Subsequently, the lysates were fractionated in 12.5% SDS-PAGE and electrotransferred to nitrocellulose by means of the semi-dry transfer technique (Biorad, Munich, Germany). The membranes were blocked with 5% skimmed milk and, after washing with TBS, incubated with antibodies against core protein (Breiner et al., 2001) (FIG. 20). The labeled viral proteins were then made visible by means of chemiluminescence. FIG. 20 shows that in all samples treated the ratio of hyperphosphorylated core protein to less phosphorylated or non-phosphorylated core protein has shifted toward the latter. It is noticeable that, for example, 10 nanoM PI has no effect whatsoever on the intracellular viral protein expression (FIG. 9, lane 6; FIG. 10, lane 6), but still exerts a distinct effect on the degree of phosphorylation of the core protein (FIG. 20, lane 6).

As a result of this study, it is therefore concluded that proteasome inhibitors lead to a change in the modification of the nucleocapsid protein and this is probably the base mechanism for the reduced infectivity of DHB viruses secreted from the cells treated with proteasome inhibitors.

### EXAMPLE 11

Primary Duck Hepatocytes are Relatively Resistant to the Toxic Effects of Proteasome Inhibitors

Since the UPS is involved in numerous cellular mechanisms, a complete inhibition of proteasome activity is, in the longer term, not compatible with cell vitality. However, various cell types exhibit a different sensitivity to the toxic action of proteasome inhibitors. Noticeably, rapidly dividing and/or activated cells usually have a higher sensitivity to proteasome inhibitors than resting and/or nonactivated cultures. This is the basis for the antineoplastic action of the proteasome inhibitors. In order to determine the toxicity of proteasome inhibitors in primary duck hepatocytes compared to human transformed hepatoblastoma cells (see example 12), dose limitation studies on proteasome inhibitors were carried out. To this end, primary hepatocytes were, as already described in example 7, obtained from duck embryos, seeded into 12-well microtiter plates with a density of approx. 8×10⁵/well, and cultured for 7 days. Cell vitality was checked under a light microscope. Parallel cultures were treated with increasing doses of PI (in each case 10 microM, 3 microM, 1 microM, 10 nanoM and 1 nanom) for 48 h. Approximately every 12 h, the cells were studied with regard to morphology and vitality under a light microscope. In addition, their functionality was determined by means of fluorescence vitality staining with fluorescein diacetate (FDA) (Sigma, Deisenhofen, Germany) (Yagi et al., 2001). FDA is actively and preferentially absorbed by hepatocytes and converted there to fluorescein by means of a lipase exclusively expressed in hepatocytes and causes fluorescence staining of the cytoplasm, indicating the functionality and vitality of hepatocytes. In this experiment, the treated and untreated primary duck hepatocytes were incubated with FDA-containing Williams E medium (5 microg/ml) at 37° C. for 5 min. The cells were then washed with PBS, and hepatocyte vitality was evaluated by means of an inverse epifluorescence microscope and further processed using the image processing system Openlab. FIG. 21 shows that all cultures which had been treated with up to 1 microM PI showed neither a morphological change nor indications of reduced vitality, i.e. they were indistinguishable from untreated hepatocytes (FIG. 22). In the non-hepatocytes which are always present in cultures of primary hepatocytes, distinct morphological changes such as, for example, rounded cells and intracellular vacuole formation were observed when treated with 3 microM and 10 microM PI. In contrast, hepatocyte vitality was unchanged, according to visual evaluation and fluorescence intensity. Only from a concentration of 10 microM PI upward, first signs of a toxic action also appeared in hepatocytes (FIG. 21). Similar results were observed for different PI such as, for example, lactacystin and epoxomicin.

In summary, it can be concluded that primary duck hepatocytes can tolerate relatively high concentrations of up to approx. 10 μM proteasome inhibitor, while proliferating liver carcinoma cells (see example 12) and other proliferating cells in the hepatocyte culture are much more sensitive to the toxic action of proteasome inhibitors (FIGS. 5, 21, 23, 24 and 25).

### EXAMPLE 12

Effect of Proteasome Inhibitors on Cellular Growth and Vitality of the Human Hepatoma Cells, HepG2

The human hepatoma cell line HepG2 was cultured at 37° C. in Dulbecco's modified Eagle's medium (DMEM) (GIBCOBRL, Paisley, Scotland) supplemented with 10% fetal calf serum (Biochrom, Berlin, Germany), 2 milliM L-glutamine (GIBCOBRL, Paisley, Scotland), 100 units ml⁻¹ penicillin and 100 microg ml⁻¹ streptomycin (Biochrom, Berlin, Germany). After passaging with 0.25% trypsin and 1 milliM EDTA (GIBCOBRL, Paisley, Scotland), the cells were seeded into 12-well microtiter plates with a density of 0.5×10⁶/well. After 24 h, the medium was replaced, and the cells were treated with PI, eponemycin and epoxomicin, in each case at concentrations of 10 microM, 3 microM and 1 microM as well as 100 nanoM, 10 nanoM and 1 nanom, for 48 h and then tested for their vitality by Trypan blue staining and morphologically evaluated by means of light microscopy. Up to a concentration of 10 nanoM, none of the proteasome inhibitors used was toxic and anti-proliferative (FIGS. 23, 24 and 25). Toxic symptoms appeared from a concentration of 100 nanoM upward for PI (FIG. 23) and epoxomycin (FIG. 25), while eponemycin (FIG. 24) was still tolerated well. At 1 microM and higher, all substances assayed were highly toxic (FIGS. 23, 24 and 25). In this case, effects such as rounding and detachment of the cells, degeneration and apoptosis were observed.

In summary, it can be concluded that proliferating liver carcinoma cells are much more sensitive to the toxic action of proteasome inhibitors, while primary duck hepatocytes can tolerate relatively high concentrations of up to approx. 10 microM proteasome inhibitors (FIGS. 21, 23, 24 and 25).

### EXAMPLE 13

Effect of Proteasome Inhibitors on the Secretion of HBV-Specific Antigens in Human Hepatoma Cell Lines

Primary human hepatocytes were obtained from human livers as described (Dandri et al., 2001) and infected with human HBV which had been harvested from the medium of HepG2.2.15 cells which produce infectious human hepatitis B virus (Sells et al., 1987). Parallel cultures of HepG2 cells were treated with PI for two days or left untreated. Before the treatment, the HepG2 cells were transfected as described (Kalinina et al., 2001) with replication-competent HBV genomes. In contrast to numerous HBV core-positive primary hepatocytes (analyzed by indirect immunofludrescence staining with anti-core antibodies) which indicate successful infection of primary human hepatocytes with viruses from the untreated cells, no core-positive primary human hepatocytes were found after infection with progeny viruses from proteasome inhibitor-treated HepG2 cells. As a result of this experiment, it can therefore be concluded that, similarly to the action on DHBV, proteasome-inhibitor treatment likewise blocks the release and infectivity of HBV.

In general, late processes of the replication cycle comprise the de novo synthesis of viral structural proteins in the infected cell, correct folding and modification, and transport of said structural protein to the site of virus assembly, followed by virus release on the cell membrane and proteolytic processing of the Gag polyproteins in the maturing virus particle by the viral protease. It was demonstrated by way of the example of the human immunodeficiency viruses as well as hepatitis B viruses that the various inhibitors of the 26S proteasome reduce both the release of virus particles and the infectivity of the released virus particles. The biochemical studies show that proteasome inhibitors block specifically the maturation and proteolytic processing of the Gag proteins and, in hepatitis B viruses, also alter post-translational modification of the nucleocapsid and reduce secretion of the e-antigen. Morphological studies show that, in the case of HIV-1, immature virus particles concentrate at the cell membrane in the presence of proteosome inhibitors. Virological studies show that proteasome inhibitors prevent the spread of an HIV-1 infection as well as an HBV infection in the cell culture. Furthermore, studies on the mechanism show that proteasome inhibitors exert no direct effect on the viral protease of HIV-1 but influence cellular mechanisms which are required for efficient maturation and release of virus particles.

The use of proteasome inhibitors represents a novel method for the intervention of viral replication cycles. Since the target of said method is conserved cellular mechanisms, i.e. the enzymic activity of the proteasome complex itself, no resistance mechanisms mediated by viral mutations are to be expected in the case of an in vivo administration of proteasome inhibitors.

The invention furthermore relates to the use in basic research, for example analyzing the assembly, release and maturation of retroviruses, especially late processes in the HIV replication cycle; further to

basic research and applied research,

the understanding of retrovirus assembly,

the understanding of the principle of action of viral proteases,

the understanding of retroviral Gag processing,

the understanding of cellular mechanisms involved in the late process of retrovirus assembly, in particular of factors of the ubiquitin-proteasome system, of ubiquitin-binding factors,

factors binding to ubiquitinated retroviral Gag proteins,

factors binding to mono-ubiquitinated L (late assembly) domains of retroviral Gag proteins,

cellular factors controlling, regulating, influencing and/or inhibiting mono-ubiquitination of retroviral L domains,

cellular factors reversing said mono-ubiquitination of L domains in retroviral Gag proteins by de-ubiquitination,

cellular factors controlling, regulating, influencing and/or inhibiting late processes of virus assembly, especially the detachment of virus particles from the cell membrane, depending on the mono-ubiquitination of L domains in retroviral Gag proteins, and

the understanding in the development of further substances which can control, regulate, influence and/or inhibit retroviral Gag processing and the assembly and release of retroviruses by influencing the interaction of retroviral Gag proteins with the ubiquitin-proteasome system.

Further fields of application of the present invention are:

the prevention and treatment/therapy of disorders caused by retroviral infection, especially anti-retroviral therapy and prevention of infections with immunodeficiency-causing lentiviruses, above all the acquired immunodeficiency in animals and humans, such as the treatment of HIV-infected individuals, symptom-free as well as AIDS patients, the prevention of an HIV infection and the prevention of an HIV infection immediately after contact with HI viruses;

the development of new pharmaceuticals, especially on the basis of substances inhibiting the 26S proteasome, such as proteasome inhibitors which can be administered in vivo and have low toxicity and, at the same time, suppress the replication of retroviruses in the organism and prevent the spread of the infection;

the application of retroviral vectors for use in gene transfer methods in gene therapy, especially the administration of proteasome inhibitors for preventing the development and spread of recombinant and/or unwanted replication-competent retroviruses after gene transfer;

virology, cell biology, gene therapy, pharmacology, organic chemistry, peptide chemistry, molecular HIV research and applied AIDS research.

### FIGURE LEGENDS

FIG. 1: Electron-microscopic analysis of HIV-1-infected CD4⁺ T cells after treatment with PI

MT-4 cells were infected with HIV-1_{NL4-3} and treated, at the time of maximum virus production (approx. 4 days post-infection) with 50 μM zLLL for 5 hours. Cells were fixed in cellulose capillaries and prepared for thin section microscopy. The panel “various budding structures” shows an overview of infected cells with viral budding structures of mature and immature viruses. The panel “mature extracellular virions” depicts mature extracellular HIV-1 particles, and the panel “arrested budding virus” depicts a high-resolution magnification of immature particles which are still connected to the cell membrane.

FIG. 2: Proteasome inhibitors inhibit Gag processing and virus release of HIV-1 and HIV-2 in infected and transfected cells

(A) HeLa cells were transfected with the HIV-1 proviral infectious DNA clone pNL4-3. After 24 hours, parallel cultures of the transfected cells were treated with proteasome inhibitors (10 microM zLLL and 10 microM LC (+INHIBITORS)) in medium or without inhibitors (NO INHIBITOR) and subjected-to a pulse/chase experiment. [³⁵S]-methionine-radiolabeled viral proteins were isolated by means of immunoprecipitation from the cellular fraction (CELL), the pelleted viral fraction (VIRUS) and the virus-free supernatant fraction obtained after centrifugation (FREE PROTEIN) and fractionated in a 10% SDS-PAGE. The radiolabeled proteins were then made visible by fluorography. The relative concentration of these proteins was quantitatively analyzed by means of image analysis. Panel “HIV-1 in HeLa” depicts representative sections of the fluorographs in the molecular weight range of CA and Gag polyprotein Pr55 (approx. 20 to 60 kDa). The positions of the main processing product p24^{CA} and of an intermediate cleavage product, p25^{CA}, are especially indicated. The kinetics of virus release were shown as the percentage of Gag proteins in the viral fraction in relation to the total amount of Gag (in CELL, VIRUS, and FREE PROTEIN) at each point in time of the chase. The kinetics of intracellular Gag processing were described as the amount of CA divided by the amount of Pr55 over the entire chase period. There is a distinct, approx. 6-fold delay in Gag processing and virus release within the 8-hour chase period. Likewise, accumulation of incomplete cleavage products such as, for example, those of p25^{CA} is clearly visible in the CELL and viral fractions. In the pulse/chase experiments relating to panel “HIV-1 in A3.01”, CD4⁺ T cells were infected with HIV-1_{NL4-3}, and the spread of the infection in the culture was monitored by determining RT activity in the cell culture supernatant. At the time of maximum virus replication, approx. 7 days after infection, parallel cultures were treated with proteasome inhibitor (10 microM of zLLL (+INHIBITOR)) in medium or without inhibitor (NO INHIBITOR) and subjected to a pulse/chase experiment, followed by immunoprecipitation and SDS-PAGE, similarly to the experiment depicted in FIG. 2, panel “HIV-1 in HeLa”. The positions of the main structural proteins of Env (gp160 and gp120) and of Gag (Pr55 and CA) are indicated in the fluorographs of the particular SDS-PAGE analyses. A distinct delay in the kinetics of intracellular Gag processing and of the release of virus-associated and free Gag, which occurs even at a relatively low concentration of 10 microM zLLL, is clearly visible. At the same time, intermediate processing products in the region of approx. 40 kDa are accumulated, in particular in the VIRUS fraction.

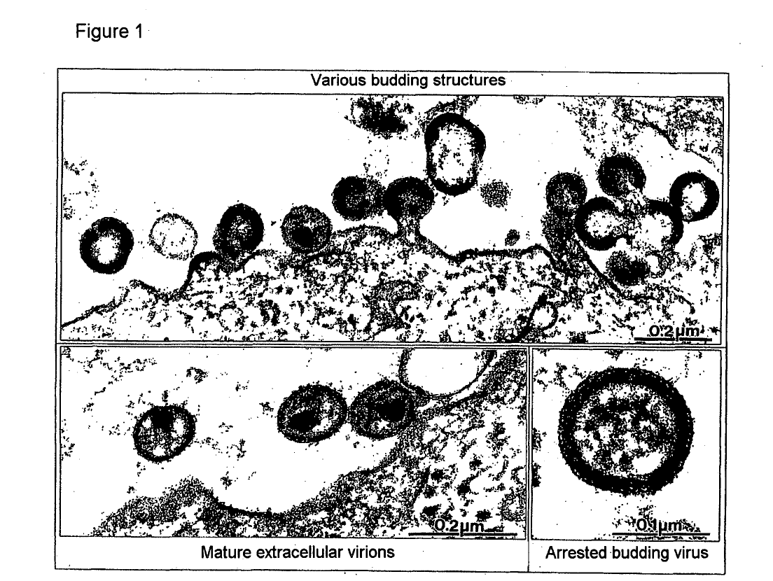

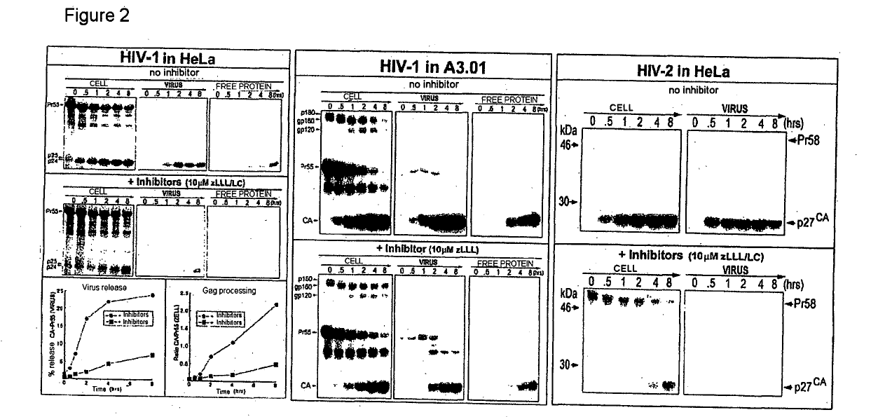

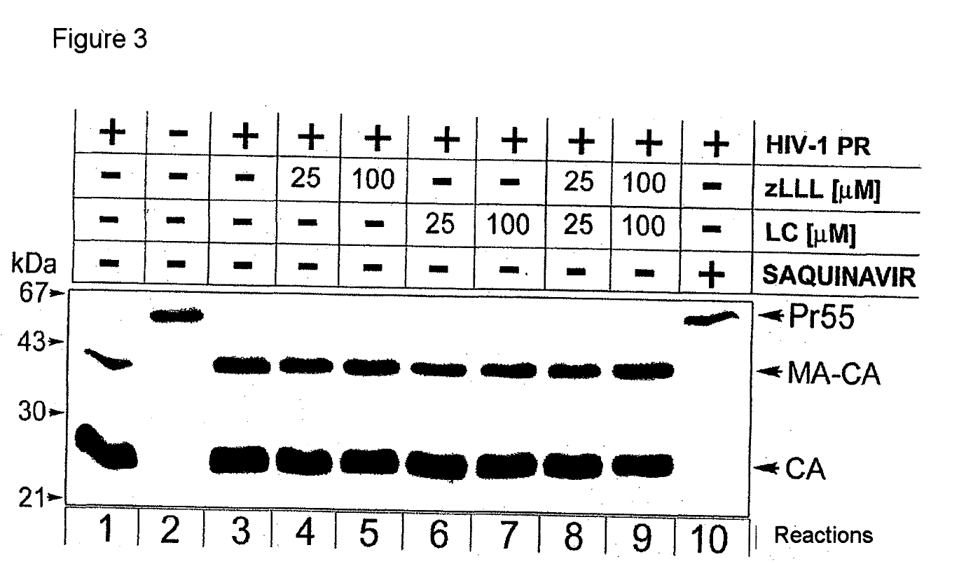

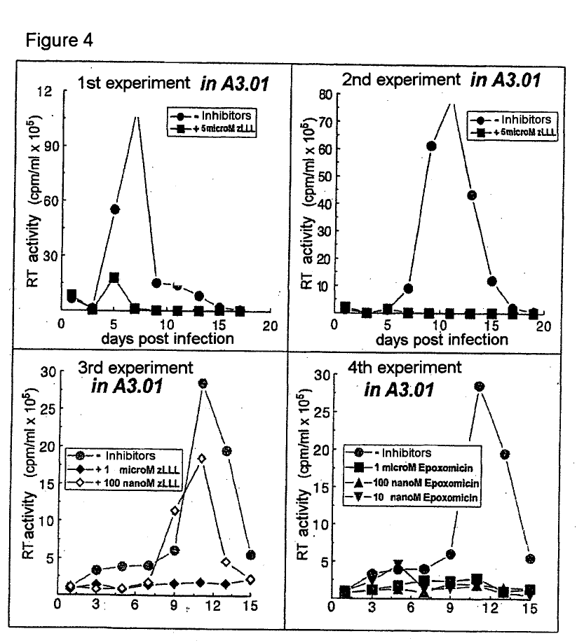

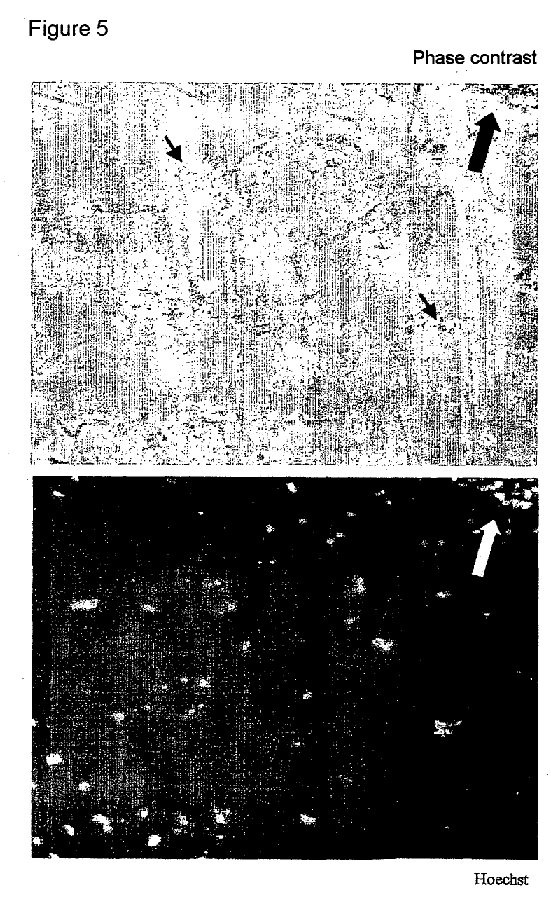

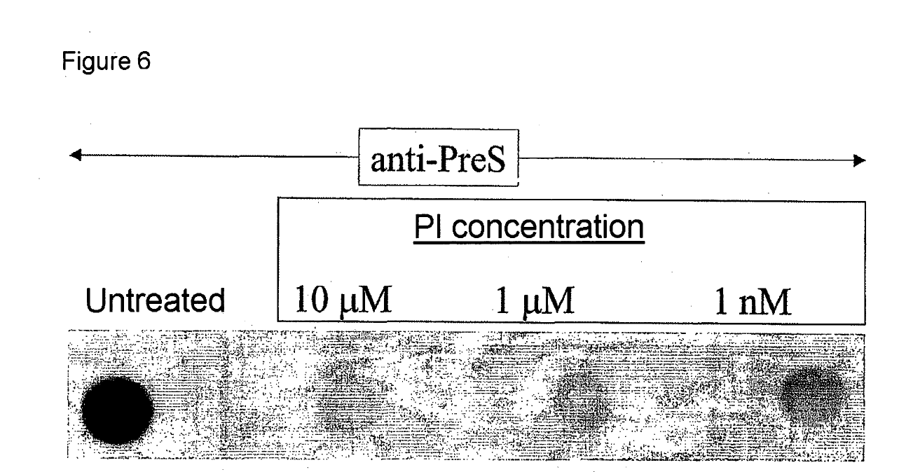

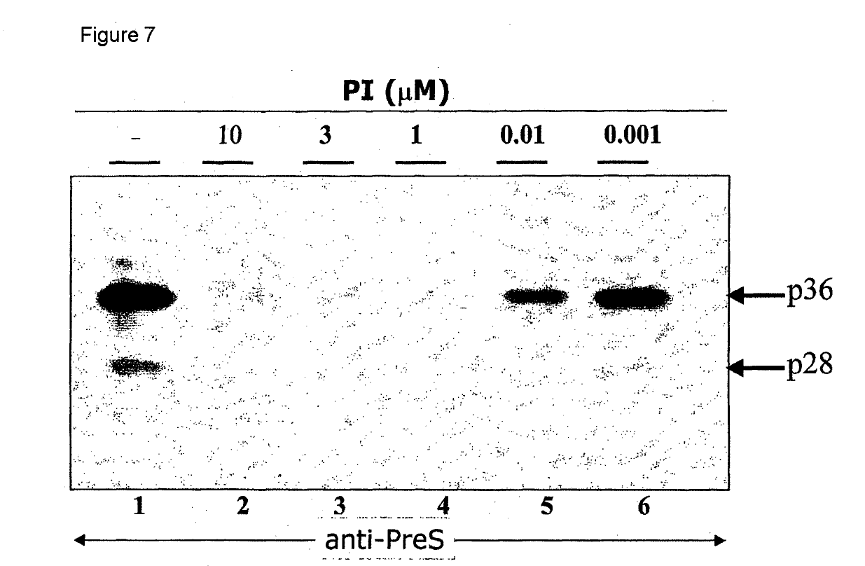

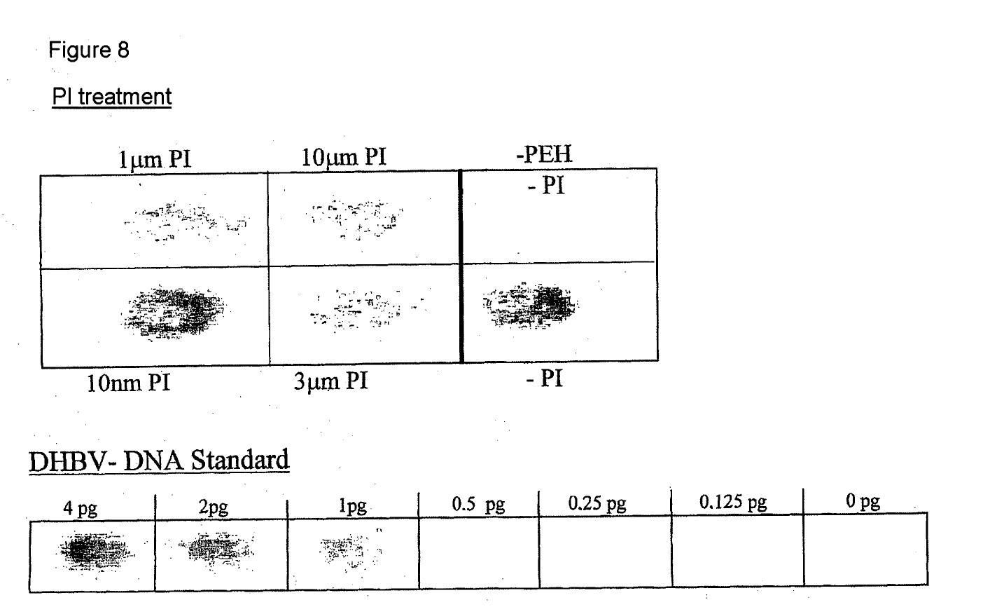

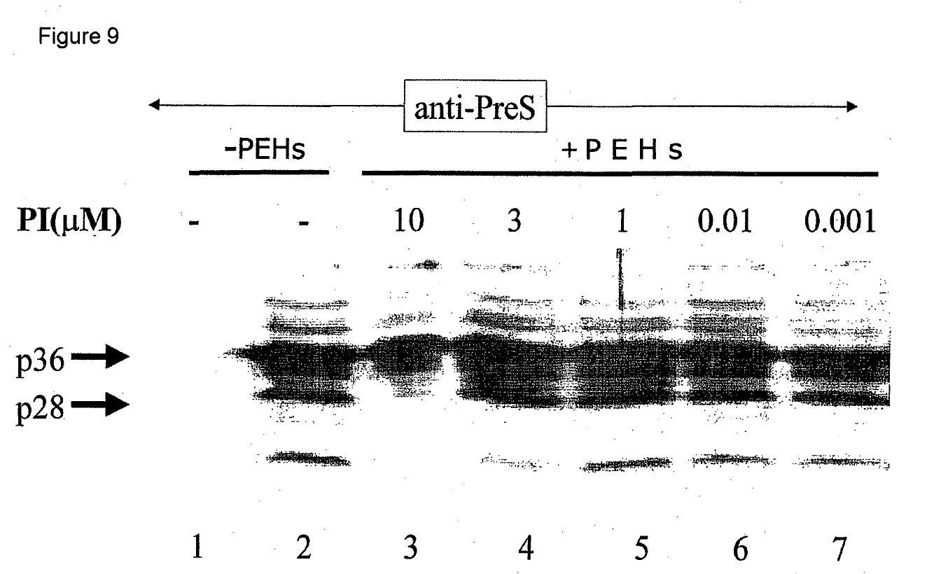

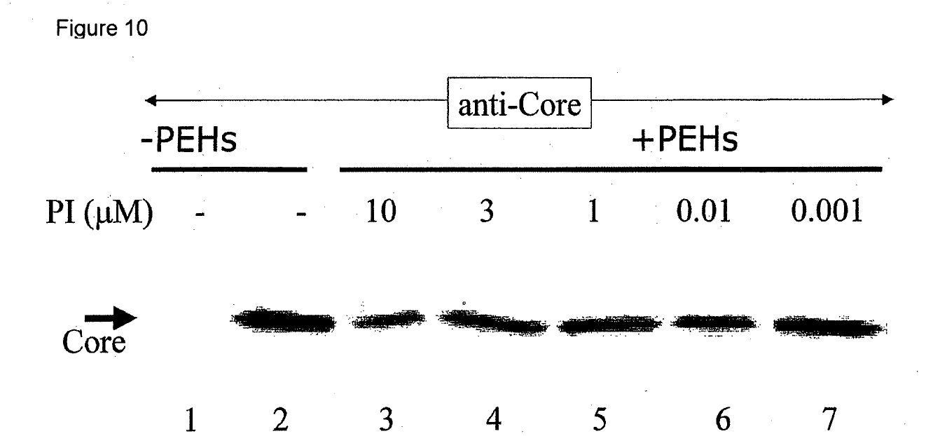

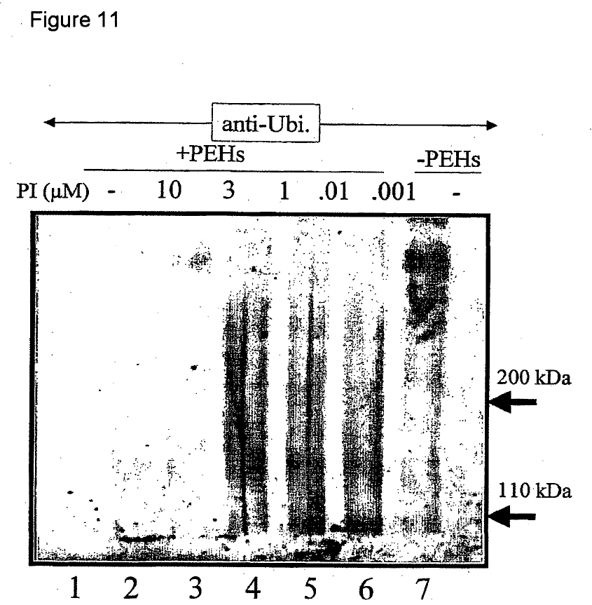

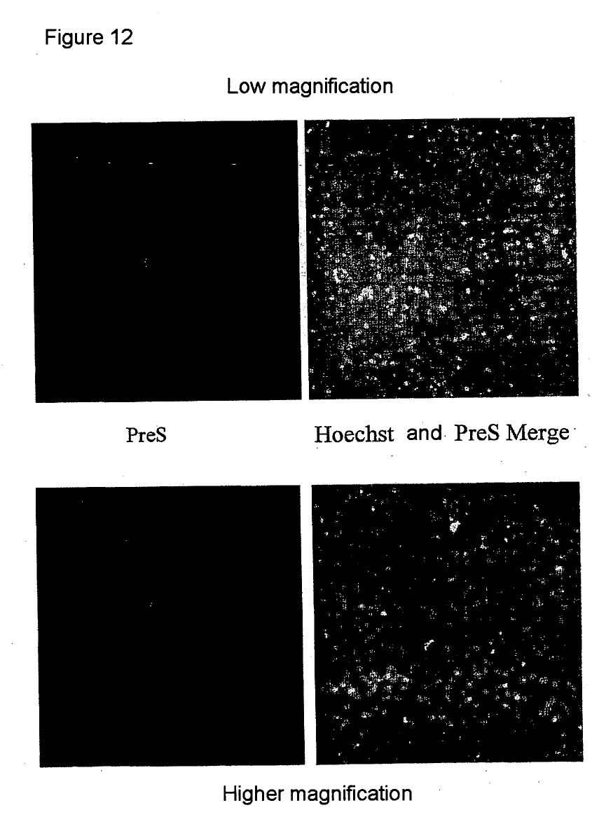

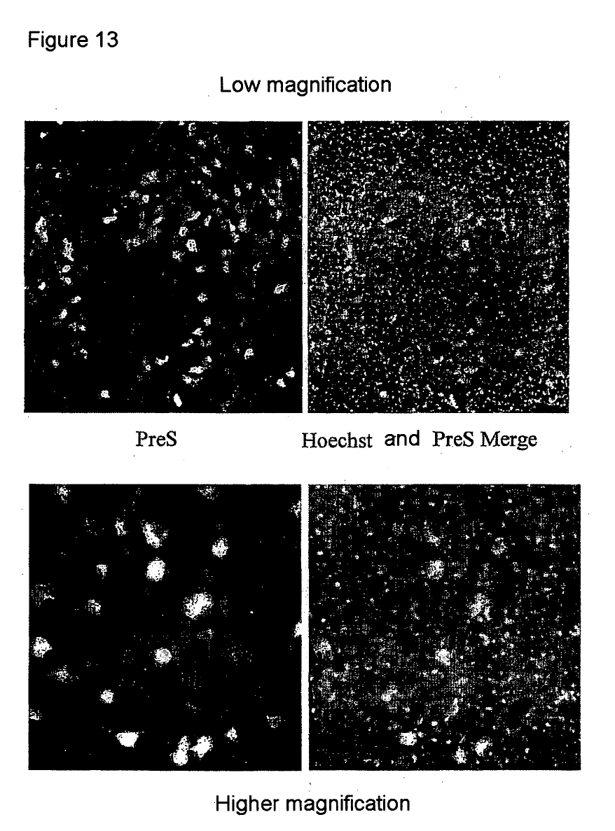

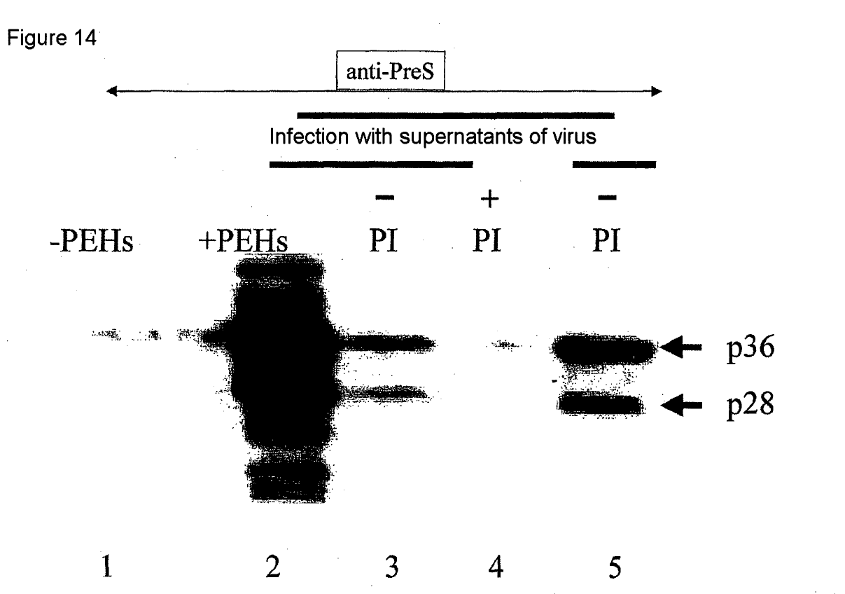

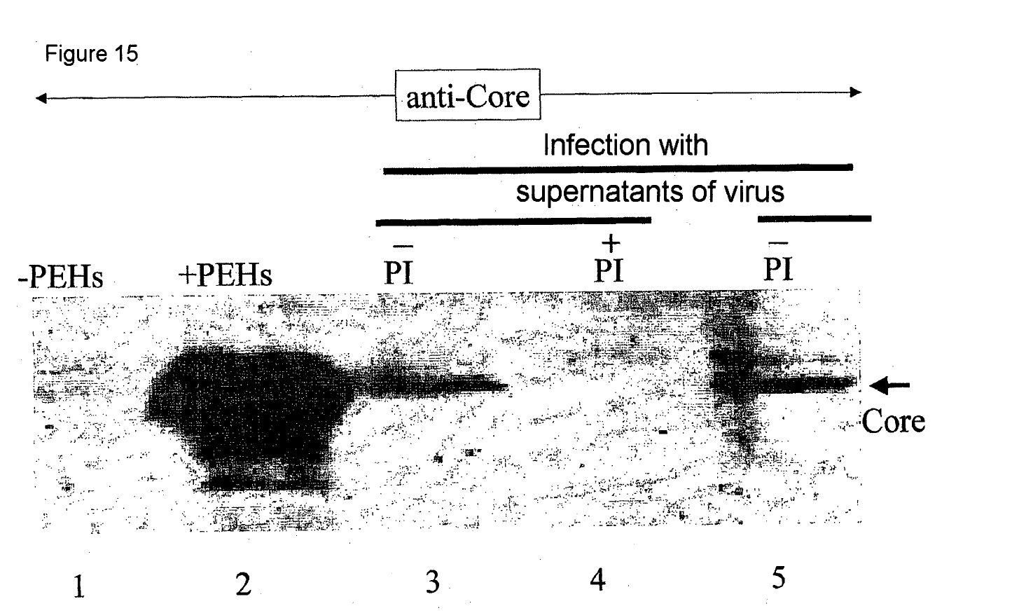

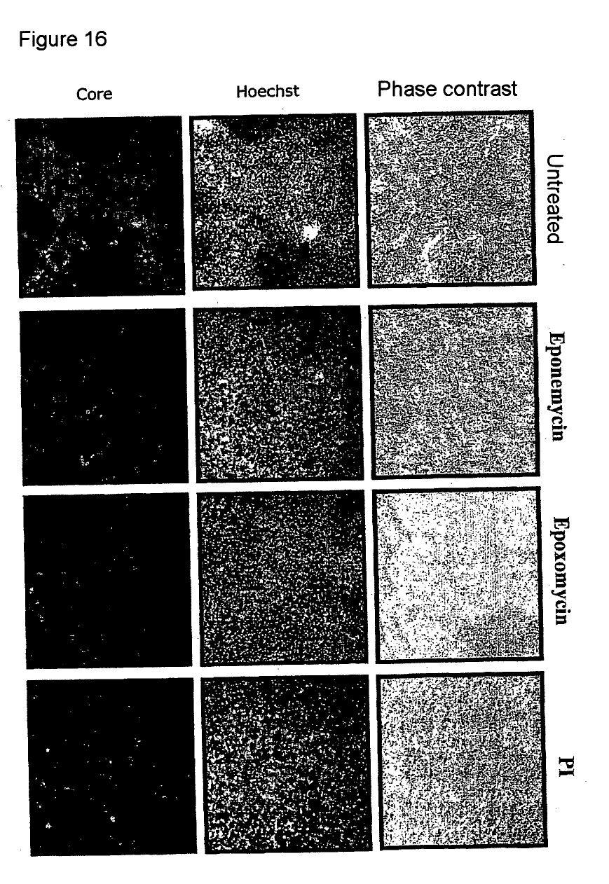

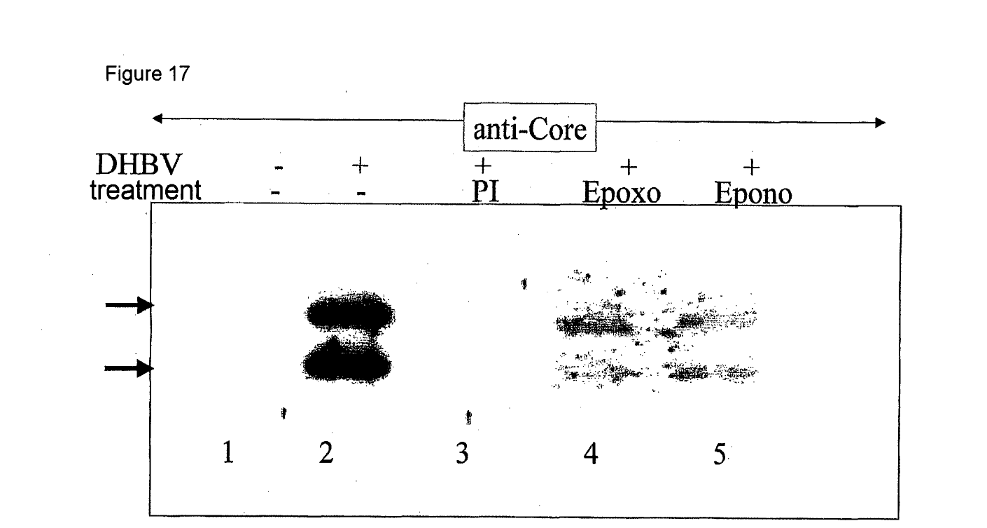

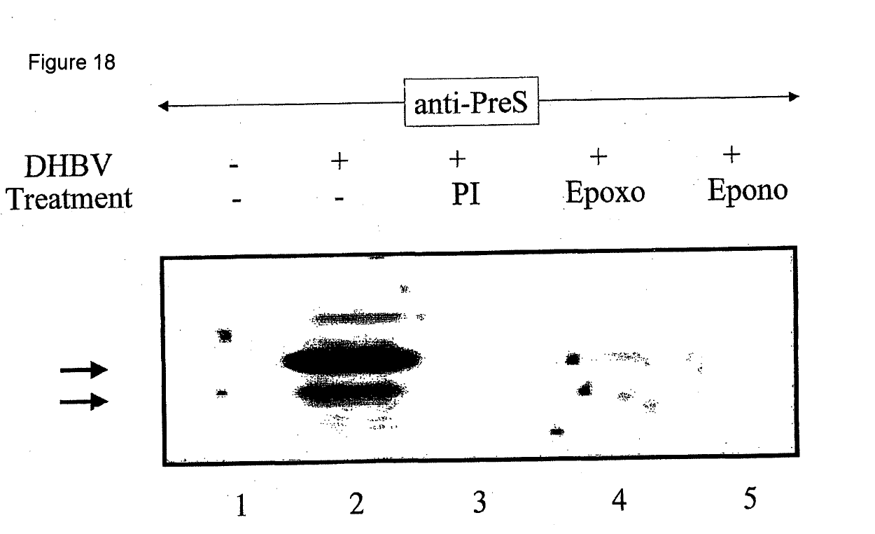

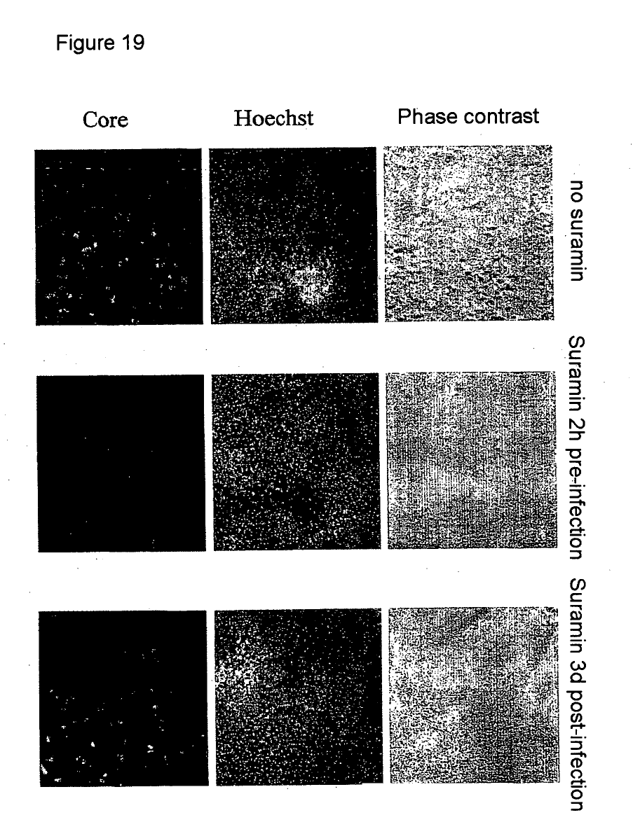

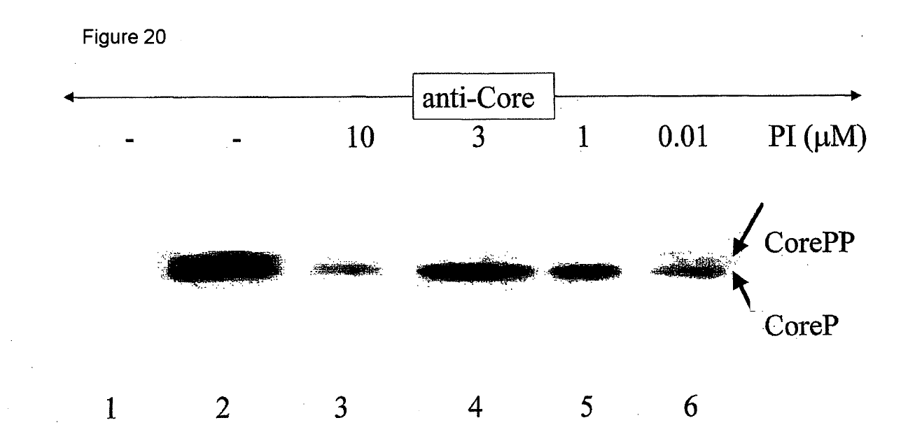

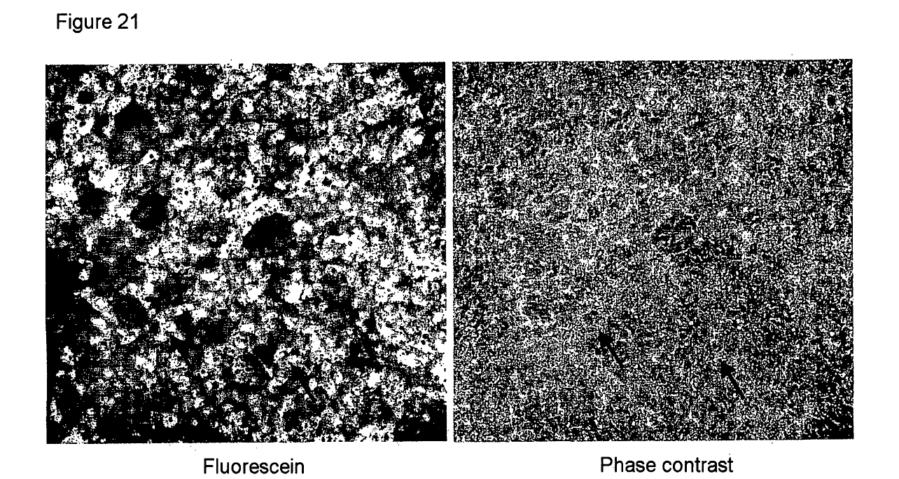

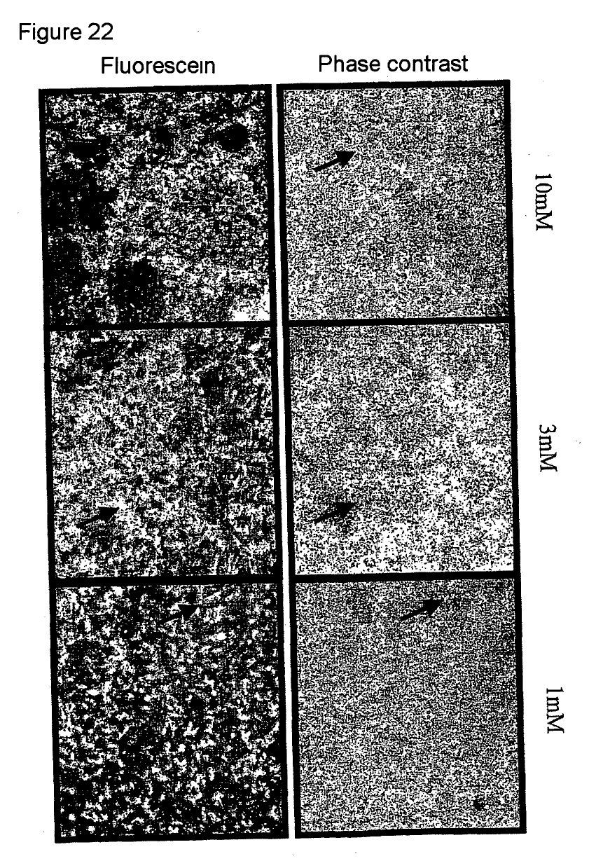

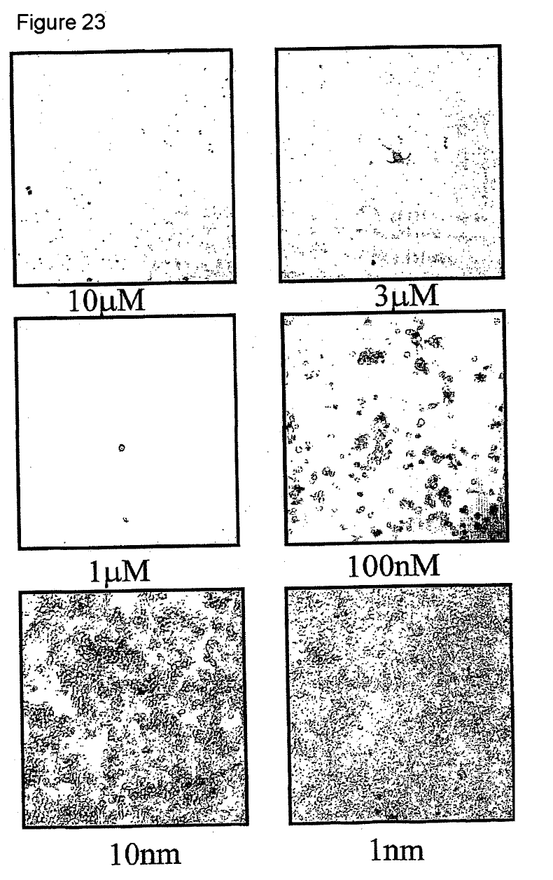

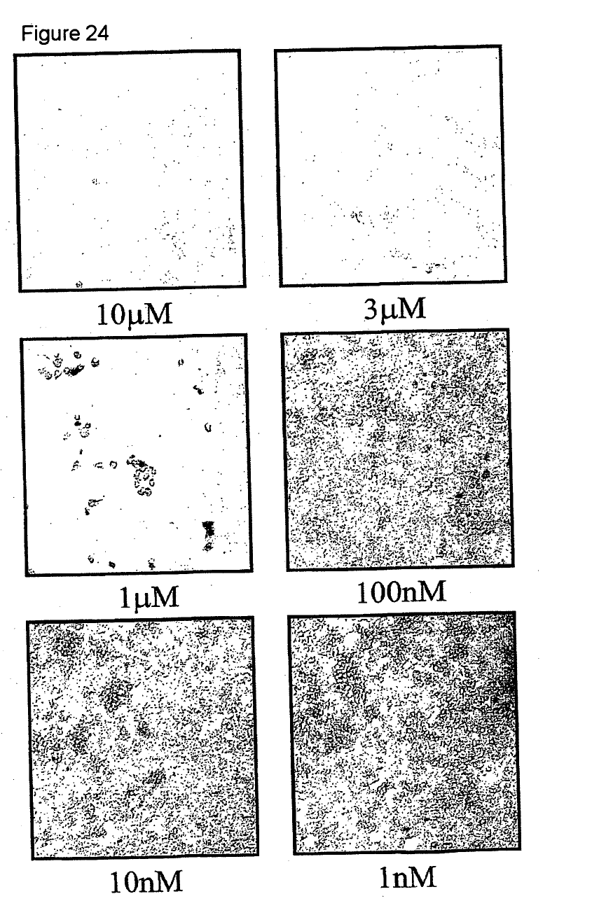

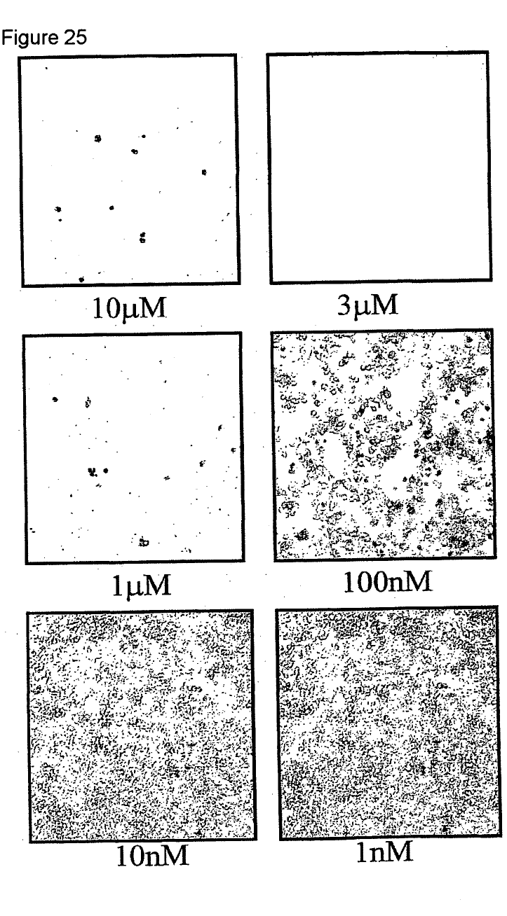

In the pulse/chase experiment relating to FIG. 2, panel “HIV-2 in HeLa”, HeLa cells were transfected with the HIV-2 proviral infectious DNA clone pHIV-2_{ROD10}. After 24 hours, parallel cultures of transfected cells were treated with proteasome inhibitors (10 microM zLLL and 10 microM LC (+INHIBITORS)) in medium or without inhibitors (NO INHIBITOR) and subjected to a pulse/-chase experiment, followed by immunoprecipitation and SDS-PAGE, similarly to the experiment depicted above in FIG. 1. FIG. 2, panel “HIV-2 in HeLa”, depicts representative sections of the fluorographs in the molecular weight range of CA and Gag polyprotein Pr55 (approx. 20 to 60 kDa) of the CELL and VIRUS fractions. Positions of the main processing products p27^{CA} and of the Gag polyprotein Pr58 are indicated. A large reduction in intracellular and virus-associated Gag processing and a distinct reduction in virus release are observed.

FIG. 3: Proteasome inhibitors have no influence on Gag processing of Pr55 by viral protease in vitro

Recombinant Pr55 was isolated from virus-like particles which had been produced by insect cells infected with recombinant bacculovirus. Recombinant HIV-1 protease was expressed in E. coli, purified, and the specific activity was determined by active-site titration. The quantities of Pr55 and protease were mixed with an enzyme-to-substrate-ratio of 1:25 and incubated at 37° C. for 30 min. The reaction was stopped by adding SDS sample buffer. The cleavage reactions were then studied by Western blot analysis with CA-specific antiserum. Pr55, CA and intermediate processing product MA-Ca were made visible, after antibody binding, by means of a -chemiluminescence reaction. In reactions 3 to 10, the protease was preincubated, prior to the start of the cleavage reaction, with the particular proteasome inhibitors for 5 min, before adding the substrate Pr55. In reaction 10, the HIV-1 protease-specific inhibitor saquinavir was added.

FIG. 4: Proteasome inhibitors inhibit HIV-1 replication in cell culture

Parallel cultures of A3.01 cells were infected with comparable infectious doses of HIV-1_{NO4-3} and treated with 5 μM (panels “1st and 2nd experiment in A3.01”). In a parallel experiment, cultures were treated with different concentrations of zLLL (0.1 and 1 μM, panel “3rd experiment in A3.01”) and epoxomicin (10 nM to 1 μM, panel “4th experiment in A3.01”). The secretion of reverse transcriptase activity into the cell culture medium was depicted in the course of the virus replication profile (approx. 2 week culture).

FIG. 5: Nonparenchymal cells of a primary hepatocyte culture are more sensitive to the toxic effects of proteasome inhibitors

A primary hepatocyte culture which has been treated with 1 microM PI for 72 h is shown. Subsequently, the cells were fixed and the nuclei stained (blue) with Hoechst. The corresponding phase contrast image is shown for comparison. The top phase contrast image depicts a former island of non-hepatocytes, while the bottom image displays the stained nuclei of the same image section. The small arrows indicate the apoptotic nonparenchymal cells, while the block arrow indicates an intact, small hepatocyte colony.

FIG. 6: Proteasome inhibitors inhibit in a concentration-dependent manner the release of PreS-containing virus particles in chronic DHBV-infected primary duck hepatocytes—PreS dot-blot detection

A 7-day-old, chronically DHBV-infected primary duck hepatocyte culture was treated with increasing PI concentrations (1 nanoM, 1 microM and 10 microM) or left untreated for 48 h. 200 microl of the particular supernatants were applied in dots. The amount of the viral particles released was determined by means of a PreS antigen-specific dot-blot.

FIG. 7: Proteasome inhibitors inhibit in a concentration-dependent manner the release of PreS-containing virus particles in chronic DHBV-infected primary duck hepatocytes—PreS Western blot detection

A 7-day-old, chronically DHBV-infected primary duck hepatocyte culture was treated with increasing PI concentrations (1 nanoM, 10 nanoM, 1 microM, 3 microM and 10 microM) or left untreated for 48 h. 5 microL of the particular supernatants were fractionated in 12.5% SDS-PAGE. The PreS-protein bands (p36 and p28) were visualized by means of Western blotting.

FIG. 8: Proteasome inhibitors inhibit in a concentration-dependent manner the release of DNA-containing virus particles in chronic DHBV-infected primary duck hepatocytes (PEH)—DNA dot-blot detection

A 7-day-old, chronically DHBV-infected primary duck hepatocyte culture was treated with increasing PI concentrations (10 nanoM, 1 microM, 3 microM and 10 microM) or left untreated for 48 h, the supernatant was collected and clarified by centrifugation. The supernatant was then applied in dots to nitrocellulose and the membrane was hybridized with a ³²P-labeled DHBV-DNA probe and subjected to autoradiography. Dots of noninfected PEHs are indicated by −, dots of infected hepatocytes are indicated by +. The DNA dot-blot was standardized by applying known concentrations of cloned DHBV DNA (DHBV-BNA standard).

FIG. 9: Proteasome inhibitors have negligible influence on intracellular PreS gene expression

7-day-old, chronically DHBV-infected primary duck hepatocyte cultures (PEHS) were treated with increasing PI doses for 48 h. Cell lysates were fractionated in 12.5% SDS-PAGE and blotted to detect PreS antigen. Lanes 1 and 2 contain untreated, noninfected (−PEHs) and chronically DHBV-infected hepatocytes (+PEHs), respectively. Lanes 3 to 7 correspond to applications of cells treated with different doses of PI-heated cells. p36 and p28 correspond to the full-length PreS protein and a degradation product thereof, respectively.

FIG. 10: Proteasome inhibitors have negligible influence on core expression

7-day-old chronically DHBV-infected primary duck hepatocyte cultures were treated with increasing PI doses for 48 h. Cell lysates were fractionated in 12.5% SDS-PAGE and the core protein (arrow) was visualized by indirect chemiluminescence. Lanes 1 and 2 contain untreated DHBV-negative (−PEHs) and chronically DHBV-infected hepatocytes (+PEH), respectively. Lanes 3 to 7 correspond to applications of cells treated with different doses of PI.

FIG. 11: Accumulation of polyubiquitinated proteins in primary duck hepatocytes treated with proteasome inhibitors

One-week-old, chronically DHBV-infected primary duck hepatocytes were treated with increasing PI concentrations (lanes 2 to 6) for 48 h. DHBV-infected (+PEHs, lane 1) and virus-free cells (−PEHs, lane 7) were cultured without PI as control. Subsequently, cell lysates were fractionated in 6% SDS-PAGE and mono- and polyubiquitinated proteins were detected by means of rabbit anti-ubiquitin. The particular position of the protein marker bands is indicated in kDA on the right-hand side. Polyubi. indicates the high-molecular polyubiquitinated proteins.

FIG. 12: Supernatants of the chronically DHBV-infected primary duck hepatocyte cultures (PEH) treated with proteasome inhibitors contain no infectious progeny viruses

7-day-old chronically DHBV-infected primary duck hepatocytes were treated with 10 microM PI for 48 h, and the harvested cell culture supernatant was then utilized for de novo infection of DHBV-negative PEH cultures. 60 h after infection, the cells were washed and fixed. Finally, the cells were stained for PreS (green). The nuclei were visualized by means of Hoechst staining (blue). The top and bottom parts of the figure are low and high magnifications, respectively, of the same image section.

FIG. 13: Progeny viruses from chronically DHBV-infected primary duck hepatocytes are infectious

Cell culture supernatants of a 7-day-old, chronically infected primary duck hepatocyte culture were collected and utilized for de novo infection of DHBV-negative primary duck hepatocytes. 60 h after infection, the cells were fixed. Finally, the cells were stained for PreS (green). The nuclei were visualized by means of Hoechst staining (blue). The lower part of the figure is the higher magnification of the same image section depicted at the top.

FIG. 14: Supernatants of the DHBV-infected hepatocytes treated with proteasome inhibitors contain no infectious progeny viruses—detection by PreS blot

7-day-old chronically DHBV-infected primary duck hepatocytes (PEH) were treated with 10 microM PI for 48 h, and the supernatant was subsequently collected, clarified by centrifugation and utilized for de novo infection of PEHs. 60 h after infection, the cells were lysed and fractionated in 12.5% SDS-PAGE. The PreS protein bands were visualized by Western blot. Lanes 1 and 2 contain DHBV-negative and chronically DHBV-infected PEH, respectively. Lanes 3 and 4 correspond to PEH treated with supernatants from untreated and PI-treated cultures, respectively. Lane 5 contains PEHs infected with an MOI of 20.

FIG. 15: Supernatants of the DHBV-infected hepatocytes treated with proteasome inhibitors contain no infectious progeny viruses—detection by core blot

7-day-old chronically DHBV-infected primary duck hepatocytes (PEH) were treated with 10 microM PI for 48 h, and the supernatant was subsequently collected, clarified by centrifugation and utilized for de novo infection of PEH cultures. 60 h after infection, the cells were lysed and the lysate fractionated by 12.5% SDS-PAGE. The core protein was visualized by Western blot. Lanes 1 and 2 contain DHBV-negative and chronically DHBV-infected PEH, respectively. Lanes 3 and 4 correspond to PEH treated with supernatants from untreated and PI-treated cultures, respectively. Lane 5 contains PEHs infected with an MOI of 20.

FIG. 16: Inhibition of the primary and secondary infections by treatment with proteosome inhibitors of primary duck hepatocytes (PEH) infected with DHBV

Four-day-old cultures of PEH were infected with an MOI of 20 for 16 h. The cells were subsequently washed and cultured in new medium for another three days. On day 3 after infection, the cells were either cultured without proteasome inhibitors (untreated) or treated with in each case 1 microM eponemycin, epoxomicin or PI for another 3 days. On day 6 of infection, the cells were washed and fixed. Subsequently, the cells were stained for core protein (green). The nuclei were stained with Hoechst (blue). The phase contrast images of the particular image sections are likewise depicted.

FIG. 17: Proteasome inhibitors inhibit the release of e-antigen in DHBV-infected primary duck hepatocytes

Four-day-old primary duck hepatocytes (PEH) were infected with an MOI of 20 for 16 h. Subsequently, the cells were washed and cultured in new medium for another three days. On day 3 after infection, the cells were treated with in each case 1 microM PI (lane 3), epoxomicin (lane 4), eponemycin (lane 5) for another three days. On day 6 after infection, the supernatant was collected and clarified by centrifugation. An aliquot of the supernatants was fractionated in 12.5% SDS-PAGE. The e-antigen protein bands (arrows) were visualized by indirect chemiluminescence. Lanes 1 and 2 contain untreated negative and, respectively, chronically DHBV-infected PEH, lanes 3, 4 and 5 contain DHBV-infected PEH treated with PI, epoxomicin and eponemycin, respectively.

FIG. 18: Proteasome inhibitors inhibit the release of PreS antigen in DHBV-infected primary duck hepatocytes

Four-day-old hepatocyte cultures were infected with an MOI of 20 for 16 h. Subsequently, the cells were washed and cultured in new medium for another three days. On day 3 after infection, the cells were treated with in each case 1 microM PI (lane 3), epoxomicin (lane 4) and eponemycin (lane 5) for another three days. On day 6 after infection, the supernatant was collected and fractionated in 12.5% SDS-PAGE. The PreS protein bands (p36 and p28) were visualized in a Western blot. Lanes 1 and 2 contain untreated negative and chronically DHBV-infected PEH, respectively, DHBV-infected PEHs were applied to lane 3 (PI), lane 4 (epoxomicin), and lane 5 (eponemycin).

FIG. 19: Suramin inhibits both primary and secondary DHBV infection in primary duck hepatocytes

Four-day-old PEHs were treated with 100 microg/ml suramin 2 h prior to infection (suramin preinfection) or initially left untreated (no suramin and suramin 3d post-infection). Subsequently, all cells were infected with an MOI of 20. After incubating for 16 h, the cultures were washed and further cultured with new medium. On day 3 after infection, the medium was again replaced and the cells in “no suramin” and “suramin pre-infection” were treated without and the cells in “suramin 3d post-infection” were treated with 100 microg/ml suramin for another three days. On day 6 of the infection, the cells were washed and fixed. The cells were then stained for core protein (core). The nuclei were stained with Hoechst (Hoechst). The phase contrast images of the particular image sections are likewise depicted (phase contrast).

FIG. 20: Proteasome inhibitors induce hypophosphorylation of the intracellular core protein in chronically DHBV-infected primary hepatocytes

7-day-old chronically DHBV-infected primary duck hepatocyte cultures were treated with increasing PI doses for 48 h and then lysed. The lysates were fractionated in 12.5% SDS-PAGE and the core protein visualized by indirect chemiluminescence. Lanes 1 and 2 contain untreated negative and chronically DHBV-infected PEH, respectively. Lanes 3 to 6 correspond to the PI-treated cells. CorePP corresponds to hyperphosphorylated and CoreP to hypophosphorylated core protein.

FIG. 21: Treatment of primary duck hepatocytes with proteasome inhibitors does not restrict their vitality and functionality

7-day-old primary cell cultures from chronically DHBV-infected duck livers were treated with increasing PI concentrations (10 microM, 3 microM and 1 microM) for 48 h. At the end of this treatment period, the cells were incubated with FDA-containing Williams E medium (5 microg/ml) at 37° C. for 5 min. Subsequently, the cells were washed with PBS, and successful conversion of FDA to fluorescein in hepatocytes was evaluated by means of an epifluorescence microscope. Cells containing cytoplasm stained green were regarded as being vital and functional. The figure depicts the particular fluorescence images (fluorescein) and corresponding phase contrast images (phase contrast), with the PI dose used being indicated. The white arrows point, by way of example, to nests of nonparenchymal cells which, compared to hepatocytes, exhibit no or only limited FDA absorption and conversion.

FIG. 22: Absorption and conversion of the viability marker fluorescein diacetate to fluorescein take place preferably in hepatocytes

One-week-old primary cell cultures from chronically DHBV-infected duck livers were incubated with FDA-containing Williams E medium (5 microg/ml) at 37° C. for 5 min. Subsequently, the cells were washed with PBS and successful conversion of FDA to fluorescein in hepatocytes was evaluated by means of an epifluorescence microscope. Cells containing cytoplasm stained green were considered vital and functional (fluorescein). The phase contrast image corresponding to the same image section is likewise depicted (phase contrast). The white arrows point, by way of example, to nests of nonparenchymal cells which, compared to the hepatocytes, exhibit no or only limited FDA absorption and conversion.

FIG. 23: Treatment of hepatoma cells (HepG2) with proteasome inhibitors restricts their vitality and functionality

HepG2 cells were treated with increasing concentrations (10 microM, 3 microM and 1 microM, 100 nanoM, 10 nanoM and 1 nanoM) of PI (section A) for 48 h. At the end of this treatment period, the cells were stained with Trypan blue and evaluated using a transmissive light microscope. Cells stained dark blue were considered nonvital. The respective PI concentration is indicated in the corresponding phase contrast images.

FIG. 24: Treatment of hepatoma cells (HepG2) with eponemycin restricts their vitality and functionality

HepG2 cells were treated with increasing concentrations (10 microM, 3 microM and 1 microM, 100 nanoM, 10 nanoM and 1 nanoM) of eponemycin (section A) for 48 h. At the end of this treatment period, the cells were stained with Trypan blue and evaluated using a transmissive light microscope. Cells stained dark blue were considered nonvital. The respective eponemycin concentration is indicated in the corresponding phase contrast images.

FIG. 25: Treatment of human hepatoma cells, HepG2, with epoxomicin restricts their vitality and functionality

HepG2 cells were treated with increasing concentrations (10 microM, 3 microM and 1 microM, 100 nanoM, 10 nanoM and 1 nanoM) of epoxomicin (section A) for 48 h. At the end of this treatment period, the cells were stained with Trypan blue and evaluated using a transmissive light microscope. Cells stained dark blue were considered nonvital. The respective epoxomicin concentration is indicated in the corresponding phase contrast images.

### TABLE 1

Proteasome Inhibitors Reduce the Infectivity of Released Virus Particles

CD4+ T cells (A3.01) were infected with HIV-1NL4-3 and, at the time of maximum virus production (approx. 7 days post-infection), parallel cultures were treated either without or with 40 microM zLLL for 1 (+zLLL “−1 hr”) or 6 hours (+zLLL “−6 hr”). The cells were subsequently washed and incubated with or without 40 microM zLLL for another 4.5 hours. In a parallel culture, cells were treated with 40 microM zLLL immediately after washing (+zLLL “0 hr”). The virus-containing supernatants were collected, and the amount of CA antigen was quantified by means of ELISA. The specific infectivity was determined as infectious virus titer per nanog CA and is shown in relation to the untreated control culture (100%).

References

Adachi, A; Gentleman, H E; König, S, Folks; T, Willey, R; Rabson, A; Martin, M A (1986). Production of acquired immunodeficiency syndrome-associated retrovirus in human and non-human cells transfected with an infectious molecular clone. J. Virol. 59: 284-291.

Adams, J; Behnke, M; Chen, S; Cruickshank, A A; Dick, L R; Grenier, L; Klunder, J M; Ma, Y T; Plamondon, L; Stein, R L (1998). Potent and selective inhibitors of the proteasome: dipeptidyl boronic acids. Bioorg. Med. Chem. Lett. 8: 333-338.

Adams, J; Palomrbella, V J; Sausville, E A; Johnson, J; Destree, A; Lazarus, D D; Maas, J; Pien, C S; Prakash, S; Elliott, P J (1999). Proteasome inhibitors: a novel class of potent and effective antitumor agents. Cancer Res. 59: 2615-2622.

Adams, J; Palombella, V J; Elliott, P J (2000). Proteasome inhibition: a new strategy in cancer treatment. Investigational New drugs 18: 109-121.

Adams, J; Stein, R (1996). Novel inhibitors of the proteasome and their therapeutic use in inflammation. Annu. Rep. Med. Chem. 31: 279-288.

André, P; Gröttrup, M; Klenerman, P; de Giuli, R; Booth, B L Jr; Cerundolo, V; Bonneville, M; Jotereau, F; Zinkernagel, R M; Lotteau, V (1998). An inhibitor of HIV-1 protease modulates proteasome activity, antigen presentation, and T cell responses. Proc. Natl. Acad. Sci. USA 95: 13120-13124.

Baumeister, W; Walz, J; Zuhl, F; Seemuller, E (1998). The proteasome: paradigm of a self-compartmentalizing protease. Cell 92: 367-380.

Bogyo, M; McMaster, J S; Gaczynska, M; Tortorella, D; Goldberg, A L; Ploegh, H (1997). Covalent modification of the active site threonine of proteasomal beta subunits and the Escherichia coli homolog HslV by a new class of inhibitors. Proc. Natl. Acad. Sci. USA 94: 6629-6634.

Bour, S; Schubert, U; Peden, K; Strebel, K (1996). The envelope glycoprotein of human immunodeficiency virus type 2 enhances particle release: a Vpu-like factor? J. Virol. 70: 820-829.

Breiner, K M; Urban, S; Glass, B; Schaller, H (2001). Envelope protein-mediated down-regulation of hepatitis B virus receptor in infected hepatocytes. J. Virol. 75: 143-50.

Bruns, K; Miska, S; Chassot, S; Will, H (1998). Enhancement of hepatitis B virus infection by non-infectious subviral particles. J. Virol. 72: 1462-1468.

Bryant, J L; Gallo R C; Weichold F F (2000). Use of protease inhibitors to modulate cellular pathways, immunity and therapies associated therewith. Patent application WO 0033654.

Chen, H S; Kew, M C; Hornbuckle, W E; Tennant, B C; Cote, P J; Gerin, J L; Purcell, R H; Miller, R L (1992). The precore gene of the woodchuck hepatitis virus genome is not essential for viral replication in the natural host. J. Virol. 66: 5682-8684.

Coux, O; Tanaka, K; Goldberg, A L (1996). Structure and functions of the 20S and 26S. proteasomes. Annu. Rev. Biochem. 65: 801-847.

Dandri, M; Burda, M R; Torok, E; Pollok, J M; Iwanska, A; Sommer, G; Rogiers, X; Rogler, C E; Gupta, S; Will, H; Greten, H; Petersen, J (2001). Repopulation of mouse liver with human hepatocytes and in vitro infection with hepatitis B virus. Hepatology 33: 981-988.

Doolittle, R F; Feng, D F; McClure, M A; Johnson, M S (1990). Retrovirus phylogeny and evolution. Curr. Top. Microbiol. Immunol. 157: 1-18.

Elliott, P J; Pien, C S; McCormack, T A; Capman, I D; Adams, J (1999). Proteasome inhibition: a novel mechanism to combat asthma. J. Allergy Clin. Immunol. 104: 294-300.

Fenteany, G; Standaert, R F; Lane, W S; Choi, S; Corey, E J; Schreiber, S L (1995). Inhibition of proteasome activities and subunit-specific amino-terminal threonine modification by lactacystein. Science 268: 726-731.

Fernholz, D; Wildner, G; Will, H (1993). Minor envelope proteins of duck hepatitis B virus are initiated at internal pre-S AUG codons but are not essential for infectivity. Virology 197: 64-73.

Frankel, A; Man, S; Elliott, P; Adams, J; Kerbel, R S (2000). Lack of multicellular drug resistance observed in human ovarian and prostate carcinoma treated with the proteasome inhibitor PS-341. Clin. Cancer Res. 6: 3719-3728.

Gaczynska M, Rock K L, Goldberg A L (1993). Gamma-interferon and expression of MHC genes regulate peptide hydrolysis by proteasomes. Nature 365: 264-267.

Gardner, R C; Assinder, S J; Christie, G; Mason, G G; Markwell, R; Wadsworth, H; McLaughlin, M; King, R; Chabot-Fletcher, M C; Breton, J J; Allsop, D; Rivett, A J (2000). Characterization of peptidyl boronic acid inhibitors of mammalian 20 S and 26 S proteasomes and their inhibition of proteasomes in cultured cells. Biochem. J. 346: 447-454.

Geier, E; Pfeifer, G; Wilm, M; Lucchiari-Hartz, M; Baumeister, W; Eichmann, K; Niedermann, G (1999). A giant protease with potential to substitute for some functions of the proteasome. Science 283: 978-981.

Glas, R; Bogyo, M; McMaster, J S; Gaczynska, M; Ploegh, H L (1998). A proteolytic system that compensates for loss of proteasome function. Nature 392: 618-622.

Hanada, M; Sugawara, K; Kaneta, K; Toda, S; Nishiyama, Y; Tomita, K; Yamamoto, H; Konishi, M; Oki, T (1992). Epoxomicin, a new antitumor agent of microbial origin. J. Antibiot. (Tokyo) 45: 1746-1752.

Hershko, A; Ciechanover, A (1998). The ubiquitin system. Annu. Rev. Biochem. 67: 425-479.

Higashitsuji, H; Itoh, K; Nagao, T; Dawson, S; Nonoguchi, K; Kido, T; Mayer, R J; Arii, S; Fujita, J (2000). Reduced stability of retinoblastoma protein by gankyrin, an oncogenic ankyrin-repeat protein over-expressed in hepatomas. Nat. Med. 6: 96-99.

Hu, Z; Zhang, Z; Doo, E; Coux, 0; Goldberg, A L; Liang, T J (1999). Hepatitis. B virus X protein is both a substrate and a potential inhibitor of the proteasome complex. J. Virol. 73: 7231-40.

Lightcap, E S; McCormack, T A; Pien, C S; Chau, V; Adams, J; Elliott, P J (2000). Proteasome inhibition measurements: clinical application. Clin. Chem. 46: 673-683.

Meng, L; Kwok, B H; Sin, N; Crews, C M (1999a). Eponemycin exerts its antitumor effect through the, inhibition of proteasome function. Cancer Res. 59: 2798-2801.

Meng, L; Mohan, R; Kwok, B H; Elofsson, M; Sin, N; Crews, C M (1999b). Epoxomicin, a potent and selective proteasome inhibitor, exhibits in vitro antiinflammatory activity. Proc. Natl. Acad. Sci. USA. 96(18): 10403-10408.

Moradpour, D; Grabscheid, B; Kammer, A R; Schmidtke, G; Gröttrup, M; Blum, H E; Cerny, A (2001). Expression of hepatitis C virus proteins does not interfere with major histocompatibility complex class I processing and presentation in vitro. Hepatology 33: 1282-1287.

Murray, R Z; Norbury, C (2000). Proteasome Inhibitors as anti-cancer agents. Anticancer Drugs 11: 407-417.

Ott, D E; Coren, L V; Copeland, T D; Kane, B P; Johnson, D G; Sowder, R C 2nd; Joshinaka, Y; Oroszlan, S; Arthur, L O; Henderson, L E (1998). Ubiquitin is covalently attached to the p6Gag proteins of human immunodeficiency virus type-1 and simian immunodeficiency virus and the p12Gag protein of moloney murine leukemia virus. J. Virol. 72: 2962-2968.

Palombella, V J; Conner, E M; Fuseler, J W; Destree, A; Davis, J M; Laroux, F S; Wolf, R E; Huang, J; Brand, S; Elliott, P J; Lazarus, D; McCormack, T; Parent, L; Stein, R; Adams, J; Grisham, M B (199.8). Role of the proteasome and NF-kappaB in streptococcal cell wall-induced polyarthritis. Proc. Natl. Acad. Sci. USA 95: 15671-15676.

Phillips, J B; Williams, A J; Adams, J; Elliott, P J; Tortella, F C (2000). Proteasome inhibitor PS-519 reduces infarction and attenuates leukocyte infiltration in a rat model of focal cerebral ischemia. Stroke 31: 1686-1693.

Pugh, J C; Simmons, H (1994). Duck hepatitis B virus infection of Muscovy duck hepatocytes and nature of virus resistance in vitro. J. Virol. 68: 2487-94.

Rehermann, B; Ferrari, C; Pasquinelli, C; Chisari, F V (1996). The hepatitis B virus persists for decades after patients' recovery from acute viral hepatitis despite active maintenance of a cytotoxic T-lymphocyte response. Nat. Med. 2: 1104-1108.

Rivett, A J; Gardner, R C (2000). Proteasome inhibitors: from in vitro uses to clinical trials. J. Pept. Sci. 6: 478-488.

Rock, K L; Goldberg, A L (1999). Degradation of cell proteins and the generation of MHC class I-presented peptides. Annu. Rev. Immunol. 17: 739-779.

Schäfer, S; Tolle, T; Lottmann, S; Gerlich, W H (1998). Animal models and experimental systems in hepatitis B virus research. In R. C. Koshy, W. H. (ed.), Hepatitis B virus: Molecular mechanisms in disease and novel strategies for therapy. Imperial College Press, London, p. 51-74.

Schmidtke, G; Holzhutter, H G; Bogyo, M; Kairies, N; Groll M; de Giuli, R; Emch, S; Gröttrup, M (1999). How an inhibitor of the HIV-I protease modulates proteasome activity. J. Biol. Chem. 274: 35734-35740.

Schubert, U; Antón, L C; Bacik, I; Cox, J H; Bour, S; Bennink, J R; Orlowski, M; Strebel, K; Yewdell, J W (1998). CD4 glycoprotein degradation induced by Human Immunodeficiency Virus type 1 Vpu protein requires the function of proteasomes and the ubiquitin conjugating pathway. J. Virol. 72: 2280-2288.

Schubert, U; Clouse, K A; Strebel, K (1995). Augmentation of virus secretion by the human immunodeficiency virus type 1 Vpu protein is cell type independent and occurs in cultured human primary macrophages and lymphocytes. J. Virol. 69: 7699-7711.

Schubert, U; Antón, L C; Gibbs, J; Norbury, C C; Yewdell, J W; Bennink, J R (2000). Rapid degradation of a large fraction of newly synthesized proteins by proteasomes. Nature 404: 770-774.

Schwartz, O; Marechal, V; Friguet, B; Arenzana-Seisdedos, F; Heard, J -M (1998). Antiviral activity of the proteasome on incoming human inmmunodeficiency virus type 1. J. Virol. 72: 3845-3850.

Schwartz, A L; Ciechanover, A (1999). The ubiquitin-proteasome pathway and pathogenesis of human diseases. Annu. Rev. Med. 50: 57-74.

Sells, M A; Chen, M L; Acs, G (1987). Production of hepatitis B virus particles in Hep G2 cells transfected with cloned hepatitis B virus DNA. Proc. Natl. Acad. Sci. USA 84: 1005-1009.

Sirma, H; Weil, R; Rosmorduc, O; Urban, S; Israel, A; Kremsdorf, D; Brechot, C (1998). Cytosol is the prime compartment of hepatitis B virus X protein where it colocalizes with the proteasome. Oncogene 16: 2054-2063.

Suzuki, R; Tamura, K; Li, J; Ishii, K; Matsuura, Y; Miyamura, T; Suzuki, T (2001). Ubiquitin-mediated degradation of hepatitis C virus core protein is regulated by processing at its carboxyl terminus. Virology 280: 301-309.

Swanstrom, R; Wills, J (1997). Synthesis, assembly, and processing of viral proteins. In Retroviruses, J. Coffin, S. Hughes and H. Varmus eds. (Plainview, N.Y.: Cold Spring Harbor Press), pp. 263-334.

Teicher, B A; Ara, G; Herbst, R; Palombella, V J; Adams, J (1999). The proteasome inhibitor PS-341 in cancer therapy. Clin. Cancer Res. 5: 2638-2645.

Theodore, T S; Englund, G; Buckler-White, A; Buckler, C E; Martin, M A; Peden, K W C (1996). Construction and characterization of a stable full-length macrophage-tropic HIV type 1 molecular clone that directs the production of high titers of progeny virions. Aids Res. Hum. Retrovir. 12: 191-194.

Trautwein, C; Manns, M. (2001). Antivirale Therapie der chronischen Virushepatatis. Internist 42: 913-923.

Voges, D; Zwickl, P; Baumeister, W (1999). The 26S proteasome: a molecular machine designed for controlled proteolysis. Annu. Rev. Biochem. 68: 1015-1068.

Yagi, T; Hardin, J A; Valenzuela, Y M; Miyoshi, H; Gores, G J; Nyberg, S L (2001). Caspase inhibition reduces apoptotic death of cryopreserved porcine hepatocytes. Hepatology 33: 1432-40.

List of abbreviations

| | |
|---|---|
| A3.01 | human CD4⁺ T-cell line |
| AIDS | acquired immunodeficiency syndrome |
| Annexin-V | cell receptor annexin-five |
| ATP | adenosine 5′-triphosphate |
| BIV | bovine immunodeficiency virus |
| BLV | bovine leukemia virus |
| CA | capsid (HIV-1 p24^{CA} antigen) |
| cccDNA | completely double-stranded supercoiled DNA |
| | genome |
| CFTR | cystic fibrosis transmembrane regulator |
| D | day |
| DHB(V) | duck hepatitis B (virus) |
| DHBV | duck hepatitis B virus |
| DMEM | Dulbecco's modified Eagle's medium |
| DMSO | dimethyl sulfoxide |
| DNA | deoxyribonucleic acid |
| ECL | enhanced chemiluminescence |
| EDTA | ethylenediaminetetraacetic acid |
| EIAV | equine infectious anemia virus |
| ELISA | enzyme linked immunoabsorbent assay |
| Env | envelope |
| ER | endoplasmic reticulum |
| FDA | fluorescein diacetate |
| FIV | feline leukemia virus |
| Gag | group-specific antigen, Core retroviral |
| | protein |
| h | hour(s) |
| hr | hour(s) |
| HAART | HAART therapy (highly active antiretroviral |
| | therapy) |
| HAV | hepatitis A virus |
| HBe | hepatitis B virus e-antigen |
| HBs | HBV surface protein |
| HBV | hepatitis B virus |
| HCC | hepatocellular carcinoma |
| HCV | hepatitis C virus |
| HDV | hepatitis delta virus |
| HEPES | (N-[2-hydroxyethyl]piperazine-N′-[2-ethane- |
| | sulfonic acid]) |
| HEV | hepatitis E virus |
| HFV | hepatitis F virus |
| HGV | hepatitis G virus |
| HIV | human immunodeficiency virus |
| HIVAN | HIV-associated nephropathy syndrome |
| HIV-1_{NL4-3} | human immunodeficiency virus, infectious |
| | clone NL4-3 |
| HLS | HIV-associated lipodystrophy syndrome |
| HPV | human papilloma virus |
| HTLV | human T-cell leukemia virus |
| IFN | interferon |
| IL | interleukin |
| kb | kilobases |
| IκB | inhibitory factor IκB |
| kDa | kilodalton (molecular weight unit) |
| Ki | inhibitory constant |
| LC | lactacystin |
| L-domain | late assembly domain, in retroviral Gag |
| | proteins |
| LLnL | leucinyl-leucinyl-nor-leucinal |
| MA | matrix, (p17^{MA} protein of HIV-1) |
| MDa | megadalton |
| MG132 | N-carbobenzoxyl-L-leucinyl-L-leucinyl-L- |
| | leucinal |
| MG232 | boric acid derivative of MG132 |
| MHC | major histocompatibility complex |
| Mo-MuLV | murine leukemia virus |
| MOI | multiplicity of infection |
| MT-4-cells | human CD4⁺ T-cell line, HTLVI-transformed |
| nanoM | nanomolar (nM) |
| NC | nucleocapsid |
| NF-κB | transcription factor |
| NLVS | 4-hydroxy-5-iodo-3-nitrophenylacetyl-L- |
| | leucinyl-L-leucinyl-L-leucine vinyl sulfone, |
| | also referred to as NLVS |
| nM | nanomole |
| ocDNA | open circular form of DNA |
| p27^{CA} | capsid (HIV-2 p27^{CA} antigen) |
| PBS | phosphate buffer, phosphate-buffered saline |
| PCR | polymerase chain reaction |
| PEH | duck hepatocytes/Peking duck hepatocytes |
| PGPH | postglutamyl peptide-hydrolyzing |
| PI | proteasome inhibitor |
| Pol | polymerase (retroviruses) |
| P-Protein | hepatitis B virus-polymerase protein |
| PR | protease |
| Pr55 | precursor Gag protein of HIV-1 |
| Pr58 | precursor Gag protein of HIV-2 |
| PreS | large hepatitis B virus envelope protein, |
| | PreS |
| PS | ProScript |
| PS-273 | morpholino-CONH-(CH-naphthyl)-CONH-(CH- |
| | isobutyl)-B(OH)₂ |
| PS-293 | PS-273 enantiomer |
| PS-296 | 8-quinolylsulfonyl-CONH-(CH-naphthyl)- |
| | CONH(-CH-isobutyl)-B(OH)₂ |
| PS-303 | NH₂(CH-naphthyl)-CONH-(CH-isobutyl)-B(OH)₂) |
| PS-325 | 2-quinol-CONH-(CH-homo-phenylalanine)-CONH- |
| | (CH-isobutyl)-B(OH)₂); |
| PS-334 | CH₃—NH-(CH-naphthyl-CONH-(CH-isobutyl)- |
| | B(OH₂); |
| PS-341 | N-pyrazinecarbonyl-L-phenylalanine-L- |
| | leucine-boric acid, empirical formula: |
| | C₁₉H₂₅BN₄O₄ |
| PS-383 | Pyridyl-CONH-(CHpF-phenylalanine)-CONH-(CH- |
| | isobutyl)-B(OH)₂ |
| PS-519 | 1R-[1S, 4R, 5S]]-1-(1-hydroxy-2-methyl- |
| | propyl)-4-propyl-6-oxa-2-azabicyclo[3.2.0]- |
| | heptane-3,7-dione, empirical formula: |
| | C₁₂H₁₉NO₄ |
| PSI | N-carbobenzoxy-Ile-Glu(Obut)-Ala-Leu-H |
| RNA | ribonucleic acid |
| RSV | Rous sarcoma virus |
| RT | reverse transcriptase |
| SDS | sodium dodecyl sulfate |
| SDS-PAGE | SDS polyacrylamide gel electrophoresis |
| SIV | simian immunodeficiency virus |
| TBS | Tris-buffered saline |
| TNF | tumor necrosis factor |
| Thr | threonine |
| Tris | Tris buffer - Tris(hydroxymethyl)amino- |
| | methane |
| Ub | ubiquitin |
| UPS | Ub-proteasome system |
| Vpu | virus protein U |
| WB | Western blot |
| XXX | category XXX = references after priority |
| | date |
| zLLL | N-carbobenzoxyl-L-leucinyl-L-leucinyl-L- |
| | leucinal |

1. A composition for treating viral infections, characterized in that it contains at least one proteasome inhibitor as active component.

2. The composition as claimed in claim 1, characterized in that it is used against viruses which are released from the cell surface during the replication cycle and in that the active components as proteasome inhibitors contain in a pharmaceutical preparation substances which inhibit, regulate or otherwise influence the activities of the ubiquitin/proteasome pathway.

3. The composition as claimed in claim 1 and 2, characterized in that the proteasome inhibitors used for inhibiting the release, maturation and replication of retroviruses and of hepatitis viruses are substances which influence especially the enzymic activities of the complete 26S proteasome complex and of the free, catalytically active 20S proteasome structure not assembled with regulatory subunits.

4. The composition as claimed in claim 3, characterized in that substances are used which are taken up as proteasome inhibitors by cells of higher eukaryotes and, after cellular uptake, interact with the catalytic proteasome subunits and, in the process, block irreversibly or reversibly all or individual proteolytic proteasome activities—the trypsin-, chymotrypsin- and postglutamyl peptide-hydrolyzing activities—within the 26S or else the 20S proteasome complex.

5. The composition as claimed in claim 1 to 4, characterized in that the pharmaceutical preparations contain, in addition to proteasome inhibitors, also other compositions which influence, regulate or inhibit the cellular ubiquitin system, such as the activities of
5.1. the ubiquitin-conjugating enzymes and/or
5.2. the ubiquitin-hydrolyzing enzymes,
5.3. cellular factors interacting with ubiquitin,
5.4. cellular factors interacting with ubiquitin as
5.4.1. monoubiquitin or as
5.4.2. polyubiquitin.

6. The composition as claimed in claim 1 to 5, characterized in that the proteasome inhibitors used as substances which are administered in vivo in various forms orally, intravenously, intramuscularly, subcutaneously or in encapsulated form with or without cell specificity-carrying modifications, which have, owing to application of a particular administration regime and/or dose regime, low cytotoxicity, cause no or negligible side effects, have a relatively long metabolic half-life and a relatively slow clearance rate in the organism.

7. The composition as claimed in claim 1 to 6, characterized in that the proteasome inhibitors used are substances which
a) are isolated in native form from microorganisms or other natural sources, or
b) are derived from natural substances by chemical modifications,
c) are totally synthesized,
d) are synthesized in vivo by means of gene therapy methods,
e) are prepared by means of genetic methods in vitro or
f) in microorganisms.

8. The composition as claimed in claim 7, characterized in that the proteasome inhibitors used are substances which belong to the following substance classes:
a) naturally occurring proteasome inhibitors:
peptide derivatives containing C-terminally epoxyketone structures,
β-lactone derivatives,
aclacinomycin A (also referred to as aclarubicin),
lactacystin and chemical modified variants thereof, such as the cell membrane-penetrating variant “Clastolactacystein □-lactone”,
b) synthetically prepared proteasome inhibitors:
modified peptide aldehydes such as N-carbobenzoxy-L-leucinyl-L-leucinyl-L-leucinal (also referred to as MG132 or zLLL), its boric acid derivative MG232; N-carbobenzoxy-Leu-Leu-Nva-H (referred to as MG115; N-acetyl-L-leucinyl-L-leucinyl-L-norleucinal (referred to as LLnL), N-carbobenzoxy-Ile-Glu(Obut)-Ala-Leu-H (also referred to as PSI);
c) peptides carrying C-terminally an α,β-epoxyketone structure, furthermore vinyl sulfones such as
8.c)1. carbobenzoxy-L-leucinyl-L-leucinyl-L-leucine vinyl sulfone or
8.c)2. 4-hydroxy-5-iodo-3-nitrophenylacetyl-L-leucinyl-L-leucinyl-L-leucin vinyl sulfone (NLVS)
d) glyoxal radicals or boric acid radicals such as
8.d)1. pyrazyl-CONH(CHPhe)CONH(CHisobutyl)B(OH)₂) and
8.d)2. dipeptidyl-boric acid derivatives or
e) pinacol esters such as benzyloxycarbonyl(Cbz)-Leu-Leu-boroLeu-pinacol ester.

9. The composition as claimed in claim 7 and 8, characterized in that particularly suitable proteasome inhibitors used are the epoxyketones
9.1. epoxomicin (epoxomycin, empirical formula: C₂₈H₈₆N₄O₇) and/or
9.2. eponemycin (eponemicink empirical formula: C₂₀H₃₆N₂O₅).

10. The composition as claimed in claim 7 and 8, characterized in that particularly suitable proteasome inhibitors used are the compounds from the PS series
10.1. PS-519 as β-lactone derivative and as lactacystin derivative the compound 1R-[1S, 4R, 5S]]-1-(1-hydroxy-2-methylpropyl)-4-propyl-6-oxa-2-azabicyclo[3.2.0]-heptane-3,7-dione, empirical formula: C₁₂H₁₉NO₄ and/or
10.2. PS-314 as peptidyl-boric acid derivative the compound N-pyrazinecarbonyl-L-phenylalanine-L-leucine-boric acid, empirical formula C₁₉H₂₅BN₄O₄ and/or
10.3. PS-273 (morpholino-CONH—(CH-naphthyl)-CONH—(CH-isobutyl)-B(OH)₂) and its enantiomer PS-293, and/or
10.4. the compound PS-296 (8-quinolyl-sulfonyl-CONH—(CH-naphthyl)-CONH(—CH-isobutyl)-B(OH)₂) and/or
10.5. PS-303 (NH₂(CH-naphthyl)-CONH—(CH-isobutyl)-B(OH)₂) and/or
10.6. PS-321 as (morpholino-CONH—(CH-naphthyl)-CONH—(CH-phenylalanine)-B(OH)₂) and/or
10.7. PS-334 (CH₃—NH—(CH-naphthyl-CONH—(CH-isobutyl)-B(OH)₂) and/or
10.8 the compound PS-325 (2-quinol-CONH—(CH-homo-phenylalanine)-CONH—(CH-isobutyl)-B(OH)₂) and/or
10.9. PS-352 (phenylalanine-CH₂—CH₂—CONH—(CH-phenyl-alanine)-CONH—(CH-isobutyl)1-B(OH)₂) and/or
10.10. PS-383 (pyridyl-CONH—(CHpF-phenylalanine)-CONH—(CH-isobutyl)-B(OH)₂).

11. The use of proteasome inhibitors as claimed in claims 1 to 10 for treating viral infections.

12. The use of proteasome inhibitors as claimed in claim 11 in pharmaceutical preparations for treating infections with hepatitis viruses and retroviruses.

13. The use of proteasome inhibitors as claimed in claim 11 for inhibiting the release, maturation and replication of retroviruses and of hepatitis viruses.

14. The use of proteasome inhibitors as claimed in claim 11 to 13 for inhibiting late processes in the replication cycle of retroviruses and of hepatitis viruses.

15. The use of proteasome inhibitors as claimed in claim 11 to 14, characterized in that the assembly and release of virions from the cell surface are inhibited.

16. The use of proteasome inhibitors as claimed in claim 11 to 14, characterized in that, in the case of retroviruses, the viral protease inhibits proteolytic processing of Gag structural proteins.

17. The use as claimed in claim 11 to 14, characterized in that the release, maturation and replication of

| | |
|---|---|
| a) | spumaviruses or |
| b) | mammalian C-type oncoviruses or |
| c) | BLV (bovine leukemia virus) or |
| d) | HTLV (human T-cell leukemia virus) or |
| e) | leukemia viruses or |
| f) | RSV (Rous Sarcoma virus) viruses or |
| g) | lentiviruses |

are inhibited.

18. The use as claimed in claim 17e, characterized in that the release, maturation and replication of

| | |
|---|---|
| a) | BLV or |
| b) | HTLV-I or |
| c) | HTLV-II |

are inhibited.

19. The use as claimed in claim 17g, characterized in that the release, maturation and replication of

| | |
|---|---|
| a) | human immunodeficiency virus type 1 (HIV-1) or |
| b) | human immunodeficiency virus type 2 (HIV-2) or |
| c) | simian immunodeficiency virus (SIV) or |
| d) | feline immunodeficiency virus (FIV) |
| e) | bovine immunodeficiency virus (BIV) |

are inhibited.

20. The use of proteasome inhibitors as claimed in claim 11 to 14 for controlling/treating disorders/pathological symptoms caused by infections with retroviruses.

21. The use as claimed in claim 20 for controlling/treating disorders/pathological symptoms caused by
21.1. infections with leukemia viruses
21.2. human T-cell leukemia viruses HTLV-I and HTLV-II
21.3. infections with lentiviruses.

22. The use as claimed in claim 21.3 for controlling/treating AIDS.

23. The use as claimed in claim 22 in combination with
23.1. other anti-retroviral medicaments
23.2. blockers of reverse transcriptase and/or viral protease
23.3. anti-retroviral therapies based on gene therapy interventions
23.4. intracellular immunization
23.5. introducing genes having anti-HIV-1/HIV-2 activity into stem cells and/or peripheral CD4⁺ lymphocytes.

24. The use as claimed in claim 22 for controlling/treating AIDS in an advanced phase of the disease.

25. The use as claimed in claim 22 for preventing the onset of the disease and for reducing spreading of the infection in the organism (reduction of viral load) of symptom-free HIV-1/HIV-2-seropositive and HIV-1/HIV-2-infected individuals.

26. The use as claimed in claim 22 for treating/controlling/preventing HIV-induced dementia, in particular for preventing HIV-infection of neurones, glia and endothelial cells in capillaries of the brain.

27. The use as claimed in claim 22 for preventing establishment of a systemic HIV-1/HIV-2 infection immediately after contact with infectious viruses, for example in the case of needle puncture injuries with HIV-contaminated blood or blood products.

28. The use of proteasome inhibitors as claimed in claim 5 and 11 to 14
28.1. in basic research and applied research,
28.2. for understanding retrovirus assembly,
28.3. for understanding the action of the viral proteases,
28.4. for understanding Gag processing of retroviruses,
28.5. for understanding cellular mechanisms involved in the late process of retrovirus assembly, for example
28.5.1. of factors of the ubiquitin-proteasome system,
28.5.2. of ubiquitin-binding factors,
28.5.3. of factors binding to ubiquinated Gag proteins of retroviruses,
28.5.4. of factors binding to mono-ubiquinated L (late assembly) domains of retroviral Gag proteins,
28.5.5. of cellular factors controlling, regulating, influencing and/or inhibiting mono-ubiquitination of L domains of retroviruses,
28.5.6. of cellular factors reversing mono-ubiquitination of L domains in Gag proteins of retroviruses by de-ubiquitination,
28.5.7. of cellular factors controlling, regulating, influencing and/or inhibiting late processes of virus assembly, especially detachment of virus particles from the cell membrane, depending on mono-ubiquitination of L domains in retroviral Gag proteins,
28.6. for understanding in the development of further substances which can control, regulate, influence and/or inhibit retroviral Gag processing and assembly and release of retroviruses by influencing the interaction of retroviral Gag proteins with the ubiquitin-proteasome system.

29. The use as claimed in claim 11 to 14, characterized in that proteasome inhibitors
29.1. substantially or completely prevent the production of infectious virions from hepatitis virus-infected cells
29.2. cause inhibition of the release of virions and also a nearly complete reduction in infectivity of the released virions
29.3. suppress virus propagation and thus de novo infection of hepatocytes and thereby spreading of a hepatitis infection in vivo in the liver tissue of an infected individual.

30. The use of proteasome inhibitors as claimed in claim 11 to 14 for inhibiting the propagation of hepadnaviruses according to the mechanisms of
a) blocking/reducing the release of new virions
b) blocking/reducing the infectivity of released virions
c) blocking/reducing the spreading of infection in cultures of primary hepatocytes
d) blocking/reducing the spreading of infection in vitro in the liver tissue of an infected individual.

31. The use of proteasome inhibitors as claimed in claim 11 to 14 for
31.1. inducing the death of liver carcinoma cells
31.2. suppressing and/or preventing the development of liver cell carcinomas
31.3. therapy of patients having established liver cell carcinomas.

32. The use of proteasome inhibitors as claimed in claim 31 for treating/controlling/preventing
32.1. HBV-induced liver cirrhosis and/or
32.2. HBV-induced liver cell carcinomas
32.3. HCV-induced liver carcinomas
32.4. medication-induced liver carcinomas
32.5. genetically caused liver carcinomas
32.6. environmentally caused liver carcinomas.

33. The use of proteasome inhibitors as claimed in claim 31 and 32 for specific elimination of liver carcinoma cells which develop as a result of
33.1. HBV infection and/or
33.2. HCV infection or
33.3. corresponding coinfection with both viruses or
33.4. HDV/HBV coinfection.

34. The use of proteasome inhibitors as claimed in claim 31 to 33 for preventing the development, growth and metastasizing of liver cell tumors and for preferred destruction of liver carcinoma cells in HBV- and HCV-infected patients and animals.

35. The use as claimed in claim 11 to 14 for
35.1. modulating the expression, modification and activity of the tumor suppressor protein p53
35.2. reducing the number of infected and hepatitis virus-producing cells in the liver cell tissue
35.3. inhibiting both maintenance and persistence of an already established infection with hepatitis viruses and also of a secondary infection and thus the spreading of an infection, including blocking the spreading of an infection with hepatitis viruses in vivo.

36. The use as claimed in claim 11 to 14, characterized in that proteasome inhibitors alter the phosphorylation of hepatitis virus-specific nucleocapsid proteins and thereby reduce or block the release and infectivity of hepatitis B and C viruses.

37. The use of proteasome inhibitors as claimed in claim 1 to 10 in combination with one another
37.1. for treating and controlling hepatitides
37.2. together with therapeutics already used in the antiviral therapy of hepadnaviruses.

38. The use as claimed in claim 11 to 14 and 37 for treating coinfections with HBV and immunodeficiency viruses HIV-1 and HIV-2.

39. The use as claimed in claim 38 for treating HBV/HIV coinfections in combination with HAART therapy.

40. The use of proteasome inhibitors as claimed in claim 11 to 14 for treating infections with hepatitis viruses to prevent
40.1. reinfection with HBV in the case of liver and other organ transplants
40.2. reinfection with HBV in the case of cell therapies by administering the compositions prior to, during and after transplantation
40.3. reinfection with HBV in the case of transplanting virus-free organs to chronic virus carriers who continuously have residual viruses and can self-infect new organs, as well as in the case of transferring virus-containing organs from donors to virus-free patients
40.4. establishment of a systemic hepatitis virus infection immediately after contact with infectious virus.

41. The use of proteasome inhibitors as claimed in claim 11 to 14 for treating infections with hepatitis viruses
41.1. to prevent a hepatitis virus infection in individuals at high risk of a new infection, such as doctors, high-risk personnel in buildings with large numbers of visitors, drug addicts, travelers in regions in which hepatitis viruses are highly endemic, in the treatment of patients or for relatives of chronic virus carriers
41.2. to reduce or eliminate inflammation of the liver by means of immune system-mediated mechanisms.

42. The use of proteasome inhibitors as claimed in claim 1 to 10 for preparing compositions and/or pharmaceutical preparations for inhibiting the release, maturation and replication of hepatitis viruses and of retroviruses.

43. The use of proteasome inhibitors as claimed in claim 42 for preparing medicaments for the treatment and prophylaxis of hepatitides and of diseases caused by retroviruses.

44. The use of proteasome inhibitors as claimed in claim 42 and 43 in the case of retroviral disorders and for preparing medicaments for the treatment of HIV-induced pathological symptoms such as AIDS and AIDS-associated disorders.

45. The use of proteasome inhibitors as claimed in claim 44 for preparing medicaments for the treatment and prophylaxis of HIV-induced dementia.

46. The use of proteasome inhibitors as claimed in claim 44 for preparing medicaments for the treatment and prophylaxis of HIV-induced disruptions in lipid metabolism, especially the HLS syndrome (HIV-associated lipodystrophy syndrome).

47. The use of proteasome inhibitors as claimed in claim 44 for preparing medicaments for the treatment and prophylaxis of HIV-induced disruptions in kidney function, especially the HIVAN syndrome (HIV-associated nephropathy).
